SEQUENCE LISTING (26 sequences)

SEQ ID NO: 1 - DNA, 18 bp, Artificial Sequence
  Description of Artificial Sequence Synthetic primer
tgggacccgatgcggtta

SEQ ID NO: 2 - DNA, 20 bp, Artificial Sequence
  Description of Artificial Sequence Synthetic primer
aatcggtctttgcctgctga

SEQ ID NO: 3 - DNA, 37 bp, Artificial Sequence
  Description of Artificial Sequence Synthetic primer
cctctctctgaaatcactaagcaggagaaagattttc

SEQ ID NO: 4 - DNA, 37 bp, Artificial Sequence
  Description of Artificial Sequence Synthetic primer
gaaaatctttctcctgcttagtgatttcagagagagg

SEQ ID NO: 5 - DNA, 32 bp, Artificial Sequence
  Description of Artificial Sequence Synthetic primer
caaatgaatgatgcacgtcatggtggctggac

SEQ ID NO: 6 - DNA, 32 bp, Artificial Sequence
  Description of Artificial Sequence Synthetic primer
gtccagccaccatgacgtgcatcattcatttg

SEQ ID NO: 7 - DNA, 40 bp, Artificial Sequence
  Description of Artificial Sequence Synthetic primer
ccagtacctcatggattacaggatttgctgaaccctattg

SEQ ID NO: 8 - DNA, 40 bp, Artificial Sequence
  Description of Artificial Sequence Synthetic primer
caatagggttcagcaaatcctgtaatccatgaggtactgg

SEQ ID NO: 9 - DNA, 28 bp, Artificial Sequence
  Description of Artificial Sequence Synthetic primer
gagaagaaggataaaacacaaaaggtac

SEQ ID NO: 10 - DNA, 28 bp, Artificial Sequence
  Description of Artificial Sequence Synthetic primer
gtaccttttgtgttttatccttcttctc

SEQ ID NO: 11 - DNA, 45 bp, Artificial Sequence
  Description of Artificial Sequence Synthetic primer
gtatttcatgaaacaactgaatgatgcacatcatggtggctggac

SEQ ID NO: 12 - DNA, 45 bp, Artificial Sequence
  Description of Artificial Sequence Synthetic primer
gtccagccaccatgatgtgcatcattcagttgtttcatgaaatac

SEQ ID NO: 13 - DNA, 27 bp, Artificial Sequence
  Description of Artificial Sequence Synthetic primer
catgcattgaactgattgccaactttc

SEQ ID NO: 14 - DNA, 27 bp, Artificial Sequence
  Description of Artificial Sequence Synthetic primer
gaaagttggcaatcagttcaatgcatg

SEQ ID NO: 15 - DNA, 36 bp, Artificial Sequence
  Description of Artificial Sequence Synthetic primer
gatccaagcttcaccatgcctccaagaccatcatca

SEQ ID NO: 16 - DNA, 36 bp, Artificial Sequence
  Description of Artificial Sequence Synthetic primer
tgatgatggtcttggaggcatggtgaagcttggatc

SEQ ID NO: 17 - DNA, 33 bp, Artificial Sequence
  Description of Artificial Sequence Synthetic primer
gacttccgaagaaatattctgaacgtttgtaaa

SEQ ID NO: 18 - DNA, 33 bp, Artificial Sequence
  Description of Artificial Sequence Synthetic primer
tttacaaacgttcagaatatttcttcggaagtc

SEQ ID NO: 19 - protein, 6 aa, Artificial Sequence
  Description of Artificial Sequence Synthetic 6xHis tag
HHHHHH

SEQ ID NO: 20 - DNA, 609 bp, Artificial Sequence
  Description of Artificial Sequence Synthetic polynucleotide
ctgaaatcactaagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcaccttttcttttggcatttaaaatctgagatgcacaataaaacagttagccagaggtttggcctgcttttggagtcctattgtcgtgcatgtgggatgtatttgaagcacctgaataggcaagttgaggcaatggaaaagctcattaacttaactgacattctcaaacaggagaagaaggataaaacacaaaaggtacagatgaagtttttagttgagcaaatgaggcgaccagact

SEQ ID NO: 21 - protein, 9 aa, Artificial Sequence
  Description of Artificial Sequence Synthetic peptide
STRDPLSEI

SEQ ID NO: 22 - DNA, 27 bp, Artificial Sequence
  Description of Artificial Sequence Synthetic oligonucleotide
tctacacgagatcgtctctctgaaatc

SEQ ID NO: 23 - protein, 9 aa, Artificial Sequence
  Description of Artificial Sequence Synthetic peptide
STRDRLSEI

SEQ ID NO: 24 - protein, 9 aa, Artificial Sequence
  Description of Artificial Sequence Synthetic peptide
MNDAHHGGW

SEQ ID NO: 25 - DNA, 27 bp, Artificial Sequence
  Description of Artificial Sequence Synthetic oligonucleotide
atgaatgatgcacgtcatggtggctgg

SEQ ID NO: 26 - protein, 9 aa, Artificial Sequence
  Description of Artificial Sequence Synthetic peptide
MNDARHGGW

(19) United States
(12) Patent Application Publication
(10) Pub. No.: US 2021/0189503 A1
(43) Pub. Date: Jun. 24, 2021

(54) BIOMARKERS FOR DETERMINING RESPONSIVENESS OF A CANCER TO PI3K INHIBITORS

(71) Applicant: MEMORIAL SLOAN-KETTERING CANCER CENTER, New York, NY (US)

(72) Inventors: Neil VASAN, New York, NY (US); Jose BASELGA, New York, NY (US); Maurizio SCALTRITI, Long Island City, NY (US)

(73) Assignee: MEMORIAL SLOAN-KETTERING CANCER CENTER, New York, NY (US)

(21) Appl. No.: 17/182,700

(22) Filed: Feb. 23, 2021

Related U.S. Application Data
(63) Continuation of application No. PCT/US2019/047879, filed on Aug. 23, 2019.
(60) Provisional application No. 62/746,959, filed on Oct. 17, 2018.
(60) Provisional application No. 62/722,046, filed on Aug. 23, 2018.

(51) Int. Cl. C12Q 1/6886

(52) U.S. Cl. CPC C12Q 1/6886; C12Q 2600/106; C12Q 2600/156

(57) ABSTRACT

The present disclosure relates to methods for determining the responsiveness of a cancer to a PI3K inhibitor and kits relating thereof. The present disclosure also relates to methods for treating a subject having a cancer, where the cancer has been determined to be responsive to a PI3K inhibitor. In particular, the present disclosure provides combinations of two or more PI3KCA mutations as biomarkers for determining the responsiveness of a cancer cell to a PI3K inhibitor.

### CROSS-REFERENCED TO RELATED APPLICATIONS

This application is a Continuation of International Patent Application No. PCT/US19/47879, filed Aug. 23, 2019, which claims priority to U.S. Provisional Application No. 62/722,046 filed Aug. 23, 2018, and U.S. Provisional Application No. 62/746,959 filed Oct. 17, 2018, the contents of each of which are incorporated by reference in their entirety, and to each of which priority is claimed.

### GRANT INFORMATION

This invention was made with government support under grant number CA223789 awarded by the National Institutes of Health. The government has certain rights in the invention.

### SEQUENCE LISTING

The specification further incorporates by reference the Sequence Listing submitted herewith via EFS on Feb. 23, 2021. Pursuant to 37 C.F.R. § 1.52(e)(5), the Sequence Listing text file, identified as 072734_1221_SL.txt, is 7,026 bytes and was created on Feb. 23, 2021. The Sequence Listing electronically filed herewith, does not extend beyond the scope of the specification and thus does not contain new matter.

### 1. INTRODUCTION

The present disclosure relates to methods for determining the responsiveness of a cancer cell or a subject suffering from cancer to a PI3K inhibitor and kits relating thereof. The present disclosure also relates to methods for treating a subject having a cancer with a PI3K inhibitor, where the subject has been determined to be likely to respond to the PI3K inhibitor. In particular, the present disclosure provides use of two or more PI3KCA mutations for determining the responsiveness of a cancer cell or a subject suffering from cancer to a PI3K inhibitor.

### 2. BACKGROUND

Over 40% of ER+MBCs are driven by activating mutations in PIK3CA, the gene coding for the catalytic subunit (p110α) of the PI3K complex, also known as phosphatidylinositol-4,5-bisphosphate 3-kinase catalytic subunit alpha. p110α binds to the regulatory subunit p85α and phosphorylates the lipid PIP2 to PIP3 resulting in recruitment of AKT and activation of signaling effectors important for cell growth and proliferation. PIK3CA E545K and H1047R are “major” hotspot mutations that hyperactivate PI3K and drive oncogenicity. PI3K inhibitors have shown encouraging results in patients with PIK3CA mutated cancers, and are now being tested in phase 3 clinical trials in ER+MBC in combination with anti-endocrine therapy. However, responses are generally modest even in patients with PIK3CA mutant tumors, suggesting that additional genomic factors may modulate the effects of PIK3CA single mutations. Previous studies have shown other PIK3CA “minor” hotspot mutations that activate PI3K to a lesser degree than E545K or H1047R; however, despite their high total frequency their mechanisms of activation and responses to therapy are less understood. Thus, there is still a need for novel methods for predicting the responsiveness of cancer cells or subjects to PI3K inhibitors.

### 3. SUMMARY OF THE INVENTION

The present disclosure relates to methods for determining the responsiveness of a cancer cell or a subject suffering from cancer to a PI3K inhibitor and kits relating thereto. The present disclosure also relates to methods for treating a subject having a cancer (e.g., a breast cancer), where the subject has been determined to be likely to respond to a PI3K inhibitor. In particular, the present disclosure provides use of two or more PI3KCA mutations for determining the responsiveness of a cancer cell or a subject suffering from cancer to a PI3K inhibitor.

The present disclosure provides methods for predicting the responsiveness of a subject suffering from a cancer to a PI3K inhibitor. In certain embodiments, the method comprises determining the presence of two or more PIK3CA mutations in a sample from the subject, wherein the presence of the two or more PIK3CA mutations indicates that the subject is more likely to be responsive to a PI3K inhibitor.

Furthermore, the present disclosure provides methods for identifying a subject suffering from a cancer as more likely to respond to a PI3K inhibitor. In certain embodiments, the method comprises determining the presence of two or more PIK3CA mutations in a sample from the subject, wherein the presence of the two or more PIK3CA mutations indicates that the subject is more likely to be responsive to a PI3K inhibitor.

In certain embodiments, the cancer is selected from the group consisting of biliary tree cancer, hepatocellular carcinoma, cancers of the head and neck, gastric cancer, endometrial carcinoma, breast cancer, brain cancer, colorectal cancer, uterine cancer, bladder cancer, lung cancer, liver cancer, glioma, head and neck cancers, stomach cancer, cervical cancer, prostate cancer, prostate adenoma, melanoma, cutaneous melanoma, upper tract urothelial cancers, esophageal cancer, esophageal squamous cell carcinoma, esophageal adenocarcinoma, cutaneous squamous cell cancers, rectal cancer, rectal adenoma, ampullary cancer, cancer of unknown primary, oropharynx squamous cell cancer, intrahepatic cholangiocarcinoma, cholangiocarcinoma, esophagogastric adenocarcinoma, mucinous carcinoma, anaplastic astrocytoma, astrocytoma, kidney cancer, papillary renal cell carcinoma, ovarian cancer, high-grade serous ovarian cancer, poorly differentiated thyroid cancer, thyroid cancer, nasopharyngeal cancer, medulloblastoma, salivary duct cancer, non-seminomatous germ cell tumor, basaloid penile squamous cell cancer, and penile cancer. In certain embodiments, the cancer is a breast cancer. In certain embodiments, the cancer is an estrogen receptor-positive metastatic breast cancer.

In certain embodiments, the two or more PIK3CA mutations are selected from Tables 4 and 5 disclosed herein.

In certain embodiments, the two or more PIK3CA mutations comprise a first PIK3CA mutation and a second PIK3CA mutation.

In certain embodiments, the first PIK3CA mutation is selected from Tables 4 and 5. In certain embodiments, the first PIK3CA mutation is selected from the group consisting of E542, E545, and H1047. In certain embodiments, the first PIK3CA mutation is selected from the group consisting of E542K, E545K, and H1047R.

In certain embodiments, the second PIK3CA mutation is selected from Tables 4 and 5. In certain embodiments, the second PIK3CA mutation is selected from the group consisting of E453, E726, and M1043. In certain embodiments, the second PIK3CA mutation is selected from the group consisting of E453Q, E453K, E726K, M1043I, and M1043L.

In certain embodiments, the first PIK3CA mutation is H1047R and the second PIK3CA mutation is E453Q or E453K. In certain embodiments, the first PIK3CA mutation is H1047R and the second PIK3CA mutation is E726K. In certain embodiments, the first PIK3CA mutation is E545K and the second PIK3CA mutation is E726K. In certain embodiments, the first PIK3CA mutation is E545K and the second PIK3CA mutation is M1043L or M1043I. In certain embodiments, the first PIK3CA mutation is E545K and the second PIK3CA mutation is E453Q or E453K. In certain embodiments, the first PIK3CA mutation is E542K and the second PIK3CA mutation is E726K. In certain embodiments, the first PIK3CA mutation is E542K and the second PIK3CA mutation is M1043L or M1043I. In certain embodiments, the first PIK3CA mutation is E542K and the second PIK3CA mutation is E453Q or E453K.

In certain embodiments, the presence of two or more PIK3CA mutations in the sample is determined by polymerase chain reaction (PCR).

In certain embodiments, the sample is a plasma sample. In certain embodiments, the plasma sample comprises circulating tumor DNA. In certain embodiments, the presence of two or more PIK3CA mutations in the sample is determined by DNA sequencing. In certain embodiments, the presence of two or more PIK3CA mutations in the sample is determined by single molecule DNA sequencing. In certain embodiments, the sample is a sample of the cancer.

In certain embodiments, the PI3K inhibitor is selected from the group consisting of BYL719, INK-1114, INK-1117, NVP-BYL719, SRX2523, LY294002, PIK-75, PKI-587, A66, CH5132799, GDC-0032 (taselisib), GDC-0077, and combinations thereof. In certain embodiments, the PI3K inhibitor is BYL719 or GDC-0032.

Furthermore, the present disclosure provides methods of treating a subject suffering from a cancer. In certain embodiments, the method comprises (a) identifying a subject as more likely to responsive to a PI3K inhibitor according to the method disclosed herein; and (b) administering to the subject a PI3K inhibitor.

The present disclosure provides kits for determining the responsiveness of a cancer cell or a subject suffering from a cancer to a PI3K inhibitor. In certain embodiments, the kit comprises a means for detecting two or more PIK3CA mutations, wherein the means comprises determining the presence of two or more PIK3CA mutations in a sample from the subject, wherein the presence of the two or more PIK3CA mutations indicates that the subject is more likely to be responsive to a PI3K inhibitor.

The present disclosure further provides kits for identifying a subject suffering from a cancer as more likely to respond to a PI3K inhibitor. In certain embodiments, the kit comprises a means for detecting two or more PIK3CA mutations, wherein the means comprises determining the presence of two or more PIK3CA mutations in a sample from the subject, wherein the presence of the two or more PIK3CA mutations indicates that the subject is more likely to be responsive to a PI3K inhibitor.

In certain embodiments, the kit disclosed herein further comprises one or more pairs of primers, probes or microarrays suitable for detecting two or more PIK3CA mutations.

In certain embodiments, the two or more PIK3CA mutations are selected from Tables 4 and 5 disclosed herein.

In certain embodiments, the two or more PIK3CA mutations comprise a first PIK3CA mutation and a second PIK3CA mutation.

In certain embodiments, the first PIK3CA mutation is selected from Tables 4 and 5. In certain embodiments, the first PIK3CA mutation is selected from the group consisting of E542, E545, and H1047. In certain embodiments, the first PIK3CA mutation is selected from the group consisting of E542K, E545K, and H1047R.

In certain embodiments, the second PIK3CA mutation is selected from Tables 4 and 5. In certain embodiments, the second PIK3CA mutation is selected from the group consisting of E453, E726, and M1043. In certain embodiments, the second PIK3CA mutation is selected from the group consisting of E453Q, E453K, E726K, M1043I, and M1043L.

In certain embodiments, the first PIK3CA mutation is H1047R and the second PIK3CA mutation is E453Q or E453K. In certain embodiments, the first PIK3CA mutation is H1047R and the second PIK3CA mutation is E726K. In certain embodiments, the first PIK3CA mutation is E545K and the second PIK3CA mutation is E726K. In certain embodiments, the first PIK3CA mutation is E545K and the second PIK3CA mutation is M1043L or M1043I. In certain embodiments, the first PIK3CA mutation is E545K and the second PIK3CA mutation is E453Q or E453K. In certain embodiments, the first PIK3CA mutation is E542K and the second PIK3CA mutation is E726K. In certain embodiments, the first PIK3CA mutation is E542K and the second PIK3CA mutation is M1043L or M1043I. In certain embodiments, the first PIK3CA mutation is E542K and the second PIK3CA mutation is E453Q or E453K.

In certain embodiments, the presence of two or more PIK3CA mutations in the sample is determined by polymerase chain reaction.

In certain embodiments, the sample is a plasma sample. In certain embodiments, the plasma sample comprises circulating tumor DNA. In certain embodiments, the sample is a sample of the cancer.

In certain embodiments, the PI3K inhibitor is selected from the group consisting of BYL719, INK-1114, INK-1117, NVP-BYL719, SRX2523, LY294002, PIK-75, PKI-587, A66, CH5132799, GDC-0032 (taselisib), GDC-0077, and combinations thereof. In certain embodiments, the PI3K inhibitor is BYL719 or GDC-0032.

In certain embodiments, the cancer is selected from the group consisting of biliary tree cancer, hepatocellular carcinoma, cancers of the head and neck, gastric cancer, endometrial carcinoma, breast cancer, brain cancer, colorectal cancer, uterine cancer, bladder cancer, lung cancer, liver cancer, glioma, head and neck cancers, stomach cancer, cervical cancer, prostate cancer, prostate adenoma, melanoma, cutaneous melanoma, upper tract urothelial cancers, esophageal cancer, esophageal squamous cell carcinoma, esophageal adenocarcinoma, cutaneous squamous cell cancers, rectal cancer, rectal adenoma, ampullary cancer, cancer of unknown primary, oropharynx squamous cell cancer, intrahepatic cholangiocarcinoma, cholangiocarcinoma, esophagogastric adenocarcinoma, mucinous carcinoma, anaplastic astrocytoma, astrocytoma, kidney cancer, papillary renal cell carcinoma, ovarian cancer, high-grade serous ovarian cancer, poorly differentiated thyroid cancer, thyroid cancer, nasopharyngeal cancer, medulloblastoma, salivary duct cancer, non-seminomatous germ cell tumor, basaloid penile squamous cell cancer, and penile cancer. In certain embodiments, the cancer is a breast cancer. In certain embodiments, the cancer is an estrogen receptor-positive metastatic breast cancer.

### 4. BRIEF DESCRIPTION OF THE DRAWINGS

FIG. 1 provides the distribution of PIK3CA mutation type in multiple breast cancer datasets (n=1853). The incidence of dual PIK3CA mutants across PIK3CA mutated breast cancer is ˜15%.

FIG. 2 illustrates the domain schematic of p110α protein and positions of major and minor mutations as asterisks.

FIGS. 3A-3D provide the effects of PIK3CA mutations on cell growth. FIG. 3A provides crystal violet growth proliferation assay of compound and single PIK3CA mutant MCF10A cells. FIG. 3B provides growth of single PIK3CA mutant MCF10A cells in vitro. FIG. 3C provides growth of E545K containing compound PIK3CA mutant MCF10A cells in vitro. FIG. 3D provides growth of H1047R-compound PIK3CA mutant MCF10A cells in vitro.

FIGS. 4A and 4B depict the effect of PIK3CA mutations on cell signaling. FIG. 4A provides western blots of PIK3CA mutant MCF10A cell signaling through the PI3K pathway. FIG. 4B provides western blots of PIK3CA mutant NIH-3T3 cell signaling through the PI3K pathway.

FIGS. 5A and 5B provide the effect of the PIK3CA mutation on activity of PI3K complexes. FIG. 5A provides liposome binding assay of recombinant compound and single PIK3CA-mutant PI3K (control liposomes). FIG. 5B provides liposome binding assay of recombinant compound and single PIK3CA-mutant PI3K (0.1% PIP2 liposomes).

FIGS. 6A-6G provide the effect of PI3K inhibitors on cell survival and cell signaling. FIG. 6A provides cell survival of PIK3CA-mutant MCF10A cells in response to the PI3K inhibitor BYL719. FIG. 6B provides western blots of PIK3CA mutant MCF10A cell signaling through the PI3K pathway, and on PI3Kα inhibition by BYL719. FIG. 6C provides western blots of PIK3CA mutant NIH-3T3 cell signaling through the PI3K pathway, and on PI3Kα inhibition by BYL719. FIGS. 6D and 6E provide western blot of PI3K effectors in PIK3CA mutant stably transduced MCF10A cells. The MCF10A cells were serum starved for 1 day, then exposed to DMSO (−) and alpelisib (1 μM) (+) for 1 hour (FIG. 6D) or GDC-0077 (62.5 nM) (+) for 1 hour (FIG. 6E). FIGS. 6F and 6G provide dose-response survival curves for MCF10A cell lines treated with alpelisib (FIG. 6F) or GDC-0077 (FIG. 6G) under serum starvation for 4 days. E545K-containing cis mutants (top) and H1047R-containing cis mutants (bottom) are compared to single PIK3CA mutants.

FIG. 7 shows the growth of PIK3CA-mutant and wild type and empty vector control NIH-3T3 cells in murine xenografts.

FIG. 8 depicts a mutational dose response model for PIK3CA mutated cancers.

FIGS. 9A-9F illustrate that dual PIK3CA-mutant tumors are frequent across all cancers including breast cancer. FIG. 9A provides a plot showing number and frequency of multiple PIK3CA mutant tumors among all PIK3CA mutant tumors across different histologies (cBioPortal). FIG. 9B illustrates codon enrichment analysis of significantly recurrent PIK3CA amino acid mutations in multiple PIK3CA mutant breast tumors (left) and non-breast tumors (right) (cBioPortal). All labeled samples are those with an fdr corrected p-value (qval)<0.01. FIG. 9C provides a Venn diagram of overlapping recurrent PIK3CA second site mutations in multiple PIK3CA-mutant breast tumors (cBioPortal and MSK-IMPACT). FIG. 9D shows clonality analysis (FACETS) of multiple PIK3CA-mutant breast tumors (Razavi, Cancer Cell 2018, 34. 427-438 dataset). Data are mean±95% confidence interval, ****p<0.0001 by two-sided Fisher's exact test. FIG. 9E illustrates bar chart of frequency of multiple PIK3CA—mutant breast tumors among primary vs metastatic cancers and by receptor subtype (NS not significant, **p<0.01) (Razavi, Cancer Cell 2018, 34. 427-438 dataset). FIG. 9F provides a list of the most frequent double PIK3CA mutation combinations in breast cancer (data from cBioPortal and MSK IMPACT). Major mutations on the left, minor mutations on the right.

FIGS. 10A-10G show the frequency of dual PIK3CA-mutant tumors across all cancers including breast cancer. FIG. 10A shows bubble plot of the number and frequency of multiple PIK3CA-mutated tumors among PIK3CA-mutant tumors (MSK-IMPACT). FIG. 10B shows a chart of the number and frequency of multiple PIK3CA-mutated tumors among PIK3CA-mutant breast tumors (cBioPortal breast datasets). FIG. 10C provides pie charts showing frequency of dual PIK3CA-mutated tumors among multiple PIK3CA-mutated tumors across various datasets. FIG. 10D provides codon enrichment analysis of amino acid positions most recurrently found in multiple PIK3CA-mutated tumors as compared to single PIK3CA-mutant tumors, among PIK3CA-mutant breast tumors (top) and non-breast tumors (bottom) (MSK-IMPACT). All labeled samples are those with an fdr corrected p value (qval)<0.01. FIG. 10E provides variant allele frequencies of dual PIK3CA mutations among the 12 most frequent histologies of PIK3CA-mutant tumors in cBioPortal. Variant allele frequencies in non-breast tumors are also shown. Plots were fitted to a 1:1 distribution, with p correlation coefficient and p-value indicated. FIG. 10F shows bar plot showing number and frequency of multiple PIK3CA mutant tumors among all PIK3CA mutant tumors across different histologies (MSK-IMPACT). FIG. 10G illustrates 2×2 tables showing frequency of double PIK3CA mutant breast tumors from cBioPortal and MSK-IMPACT with major mutations E542, E545, or H1047 (boxed in red) and with minor mutations E453, E726, or M1043. Tumors containing major mutations are box on top, and minor mutations are boxed on the left

FIGS. 11A-11D show that dual PIK3CA mutations in breast cancer are compound mutations, in cis on the same allele. FIG. 11A provides Sanger sequencing tracing from cDNA from PIK3CA dual mutant breast tumor (E545K/E726K). Compound mutations were found in 14/14 (100%) mutant clones. FIG. 11A discloses Seq ID NO: 20. FIG. 11B shows workflow for SMRT sequencing from fresh frozen tumors. FIG. 11B discloses Seq ID NOs: 1 and 2, respectively, in order of appearance. FIG. 11C illustrates SMRT-seq phasing of allelic configuration of six PIK3CA dual mutant breast tumors (E545K/E726K, E545K/T1025A, E545K/M1043L, E453K/H1047R, E542K/E726K, P539R/H1047R). Compound mutations are shown as two vertical colored squares, wildtype sequences are shown as two vertical black squares, and single mutations are shown as single colored squares (yellow or green), in order of the frequency of amplicons. Compound mutations were found in 6/6 (100%) fresh breast tumors. FIG. 11D provides table showing recurrent double PIK3CA mutations, distances in genomic DNA (gDNA) and complementary DNA (cDNA), and resolution abilities by different sequencing techniques from FFPE archival and fresh tumors. In the first column, major mutations are enlisted before minor mutations. Double mutants resolvable by SMRT-seq are bolded.

FIGS. 12A-12B depict double PIK3CA mutations in cis on the same allele. FIG. 12A shows Sanger sequencing tracing from cDNA from BT20 breast cancer cell line (P539/H1047R). Two separate priming reactions are denoted from cDNA from the same single colony. Compound mutations were found in 13/14 (93%) mutant clones, H1047R single mutation was found in 1/14 (7%) mutant clones, and P539R single mutation was found in 0/14 (0%) mutant clones. FIG. 12A discloses SEQ ID NOs: 21-26, respectively, in order of appearance. FIG. 12B illustrates SMRT-seq phasing of allelic configuration of four PIK3CA dual mutant breast cancer cell lines (BT20 [P539R/H1047R], CAL148 [D350N/H1047R], HCC202 [E545K/L866F], MDA-MB-361 [E545K/K567R]). Compound mutations are shown as two vertical colored squares, wildtype sequences are shown as two vertical black squares, and single mutations are shown as single colored squares (yellow or green), in order of the frequency of amplicons.

FIGS. 13A-13H illustrate compound PIK3CA mutations constitutively activating the PI3K pathway more than single hotspot PIK3CA mutants. FIG. 13A provides crystal violet assay of compound and single PIK3CA-mutant stably transduced MCF10A cells under serum starvation for 4 days (representative sample shown, n=3). FIG. 13B shows growth proliferation of compound and single PIK3CA-mutant stably transduced MCF10A cells under serum starvation (without EGF or insulin). Cells were developed using crystal violet assays and growth was normalized to the OD595 at day 0 for each construct (n=3, mean and SEM shown). FIG. 13C provides western blotting of PI3K effectors of compound and single PIK3CA-mutant stably transduced MCF10A cells. MCF10A cells were under serum starvation for 1 day. FIG. 13D shows western blotting of PI3K effectors of compound and single PIK3CA-mutant stably transduced NIH-3T3 cells. NIH-3T3 cells were under serum starvation for 1 day. FIG. 13E illustrates NIH-3T3 murine xenograft tumor growth of E726K/H1047R compound mutant compared to H1047R, E726K, wildtype, and empty vector (n=4 in each arm, mean and SEM shown). FIG. 13F provides western blotting for PI3K effectors of E726K/H1047R compound mutant, H1047R, E726K, wildtype, and empty vector NIH-3T3 derived murine xenograft tumors. FIG. 13G shows immunohistochemistry for pAKT (S473) of E726K/H1047R compound mutant, H1047R, E726K, wildtype, and empty vector NIH-3T3 derived murine xenograft tumors. FIG. 13H illustrates western blotting of PI3K effectors of PIK3CA mutant MCF10A cells, serum starved for the indicated time points.

FIGS. 14A-14D show cellular assays of PIK3CA mutations. FIG. 14A provides western blotting of PI3K effectors of compound and single PIK3CA-mutant stably transduced MCF7 cells (in a PIK3CA wildtype background) under serum starvation for 1 day. FIG. 14B provides western blotting of PI3K effectors of PIK3CA mutant stably transduced NIH-3T3 cells, serum starved for 1 day, then stimulated with PDGF-BB (20 ng/mL, 30 minutes) (top) or IGF-1 (10 nM, 10 minutes) (bottom). FIG. 14C provides western blotting of PI3K effectors of E726K/H1047R in cis, in trans, and single PIK3CA mutant MCF10A cells serum starved for 1 day. FIG. 14D provides crystal violet assay of PIK3CA mutant MCF10A cells under serum starvation for 4 days (representative sample shown, n=3).

FIGS. 15A-15I depict the effect of compound PIK3CA mutations promoting a more open PI3Kα conformation and more lipid binding than single mutants. FIG. 15A shows thermal shift assays of compound and single mutant recombinant full length PI3K complexes, blotted for p110α (representative blots from one experiment, n=3). All compound mutants are compared individually to their constituent single mutants and wildtype control. FIG. 15B shows thermal shift assays of major and minor single mutant recombinant full length PI3K complexes, blotted for p110α (representative blots from one experiment, n=3). FIG. 15D provides liposome binding assays compound and single mutant recombinant full length PI3K complexes, blotted for p110α (representative blots from one experiment, n=3). FIG. 15E provides domain schematic of p110α and p85α with minor and major mutation sites indicated. Colored domains correspond with reported PI3Kα crystal structures including in FIG. 15F. FIG. 15F provides crystal structure of truncated PI3K complex (PDB 4OVU) (Miller, Oncotarget, 2014, 5, 5198-5208) comprised of full length p110α and niSH2 domains of p85a, with recurrent major and minor mutation sites shown as spheres, assigned per their mechanism in FIG. 15G. Double headed arrows correspond to compound mutant combinations, assigned per their mechanism in FIG. 15G. FIG. 15G provides a table summarizing major and minor mutants, reported single mutant mechanisms, combinations of single mutations that form compound mutations, and compound mutant mechanisms per this study. FIG. 15H shows thermal shift assays of recombinant PI3K complexes. Western blot densitometry was performed, normalized to measurements of the lowest temperature, and data were fit to Boltzmann sigmoidal curves, from which the midpoint melting temperature (Tₘ 50%) was determined (n=2). FIG. 15C provides liposome sedimentation assays of cis and single p110α mutant recombinant PI3K complexes blotted for p110α with quantifications for FIG. 15I anionic liposomes and 0.1% PIP2-containing liposomes. Data are mean±s.e.m (n=3 for each).

FIGS. 16A-16F show effect of compound and single mutant on PI3K complexes activity and on the structural mapping of p110α E453 and E726 residues in PI3Kα. FIG. 16A provides in vitro lipid kinase assay of single mutant recombinant truncated PI3K complexes (full length p110α+niSH2 domains of p85a), by detection of ³²P-PIP2 (PIP3) after thin layer chromatography (TLC). FIG. 16B provides input control of normalized amounts of compound and single mutant recombinant full length PI3K complexes, blotted for p110α. FIG. 16C provides thermal shift assays of single mutant recombinant full length PI3Kα complexes as compared to wildtype control, blotted for p110α (representative blots from one experiment, n=3). FIG. 16D, left panel, provides electrostatic surface diagram of solvent-accessible area of PI3Kα, based on crystal structure of truncated PI3K complex (PDB 4ovu) comprised of full length p110α and niSH2 domains of p85α. Negatively and positively charged surfaces are denoted in red and blue, respectively. The putative positively charged membrane binding surface is shown in black box with negatively charged E726 shown in black circle. FIG. 16D, right panel, provides structure at same orientation with E726 shown as black sphere. FIG. 16E provides structural alignments of PDB 2RD0, 4OVU, and 3HHM PI3Kα crystal structures. RMSD comparisons are shown in box. FIG. 16F, left panel, provides structural alignments of PDB 2RD0, 4OVU, and 3HHM PI3Kα crystal structures in the putative membrane binding mode (as in FIG. 16D, left panel). FIG. 16F, right panel, shows E726 as sticks and magnified.

FIGS. 17A-17F illustrate that compound PIK3CA mutations exhibit more inhibition by BYL719 in cells and in patients. FIG. 17A provides western blotting of PI3K effectors of compound and single PIK3CA-mutant stably transduced MCF10A cells with BYL719 (1 μM) under serum starvation for 1 day. FIG. 17B provides western blotting of PI3K effectors of compound and single PIK3CA-mutant stably transduced NIH-3T3 cells with BYL719 (1 μM) under serum starvation for 1 day. FIG. 17C provides fold inhibition by BYL719 of E545K-containing compound and single PIK3CA-mutant stably transduced MCF10A cells to BYL719. MCF10A cells were under serum starvation for 3 days (n=3, mean and SEM shown). FIG. 17D provides fold inhibition of H1047R-containing compound and single PIK3CA-mutant stably transduced MCF10A cells to BYL719. MCF10A cells were under serum starvation for 5 days (n=3, mean and SEM shown). FIG. 17E provides overall PFS and PFS at 30-week cut-point for dual PIK3CA mutant breast cancer patients vs single PIK3CA-mutant breast cancer patients receiving BYL719 and aromatase inhibitor on phase 1 clinical trial (NCT 01870505). FIG. 17F shows a model for double hit compound PIK3CA mutations in PI3K activation and in response to PI3K inhibitor therapy.

FIGS. 18A-18C show signals of improved clinical response to PI3K inhibition in some breast cancer patients with double PIK3CA mutations. FIG. 18A provides retrospective analysis of PFS of patients with dual PIK3CA mutant, single PIK3CA mutant, and wildtype PIK3CA breast cancers on aromatase inhibitor therapy (top) or fulvestrant (bottom). Patients with both pre and post treatment biopsies confirming PIK3CA mutation from the presently disclosed cohort (n=1918) were included. FIG. 18B variant allele frequencies of the primary tumor and 14 metastases of an exceptional responder patient to alpelisib monotherapy. The plot was fitted to a 1:1 distribution, with p correlation coefficient indicated. FIG. 18C provides bar graphs of progression free survival of ER+ metastatic breast cancer patients with WT, single, and double PIK3CA mutant tumors on a phase 1 clinical trial of alpelisib and an aromatase inhibitor (7.5 weeks [95% CI 5 weeks-not reached] vs 20 weeks [95% CI 10 weeks-not reached] vs 48 weeks [95% CI 13 wks-49 weeks]). NS=not significant.

FIGS. 19A-19E show multiple PIK3CA mutations as detect by ctDNA confer increased sensitivity to taselisib compared to single PIK3CA mutations in patients. FIG. 19A shows schematic showing plasma sample acquisition from patients on the SANDPIPER clinical trial and analysis and sequencing of circulating tumor DNA (ctDNA) specimens to determine PIK3CA mutational status. FIG. 19B provides waterfall plot denoting the range of tumor shrinkage (as measured by percentage change of the sum of the longest dimensions [SLD] of target lesions compared to baseline) for individual patients with measurable disease on the taselisib arm of the SANDPIPER clinical trial, colored by ctDNA single vs multiple PIK3CA mutation status. FIGS. 19C-19E provide overall response rates (as defined by the percentage of patients with tumor shrinkage ≥30%) of placebo vs taselisib arms from the SANDPIPER clinical trial of ctDNA PIK3CA mutant total population (9.7% vs 20.3% [95% CI 4.8-16.7% vs. 15.5-25.9%, p=0.0202]) (FIG. 19C), single ctDNA PIK3CA mutant subpopulation (10.0% vs 18.1% [95% CI 4.4-18.1% vs. 13.0-24.2%, p=0.0981]) (FIG. 19D), and multiple ctDNA PIK3CA mutant subpopulation (8.7% vs. 30.2% [95% CI 1.6-26.8% vs. 18.4-44.9%, p=0.0493]) (FIG. 19E). Data are mean and 95% CI (by the Blyth-Still-Casella method) and the CI for the difference in ORRs between the two treatment arms were determined using the normal approximation to the binomial distribution. Response rates in the treatment arms were compared (p-value) using the stratified Cochran-Mangel-Haenszel test with *p<0.05, NS=not significant.

FIGS. 20A-20E illustrate the effect of PI3K pathway inhibition on PIK3CA mutations in cis. FIGS. 20A-20B provide western blotting of PI3K effectors of PIK3CA mutant stably transduced NIH-3T3 cells (FIG. 20A) and MCF7 cells (FIG. 20B). Cells were serum starved for 1 day then exposed to DMSO (−) or alpelisib (1 μM) (+) for 1 hour. FIG. 20C illustrates IC50, Emax, and AUC values for PIK3CA mutant MCF10A cells for alpelisib and GDC-0077. FIG. 20D provides dose-response survival curves for MCF10A cell lines treated with everolimus. Cells were under serum starvation for 4 days. E545K-containing cis mutants (left panel) and H1047R-containing cis mutants (right panel) are compared to single PIK3CA mutants. Data are mean±s.e.m. for triplicate cultures and were fit to asymmetric, five parameter sigmoidal curves. ****p<0.0001, ***p<0.001, **p<0.01, *p<0.05, by two-way ANOVA corrected for multiple comparisons by Tukey's test, as compared to E545K [left] or H1047R [right]). FIG. 20E illustrates dose-response survival curves for MCF10A cell lines treated with alpelisi. Cells were under serum starvation for 4 days. H1047R-containing cis mutants are compared to single PIK3CA mutants.

FIG. 21 provides clinicogenomic analysis of PIK3CA mutant breast cancers. METABRIC 2019 (Bertucci et al., Nature 569, 560-564 (2019)) and Razavi 2018 cohorts (Razavi et al., Cancer Cell 34, 427-438 e426 (2018)) were analyzed. p values were calculated by t-test (age) and chi square or Fisher's exact test, when appropriate.

FIGS. 22A-22B provides survival analysis of PIK3CA mutant HR+/HER2− breast cancer patients. FIG. 22 A: Invasive disease-free survival analysis of METABRIC 2019 cohort (Bertucci et al., Nature 569, 560-564 (2019)). FIG. 22 B: Overall survival analysis of Razavi 2018 cohort (Razavi et al., Cancer Cell 34, 427-438 e426 (2018)). For univariate analysis, p values were calculated using the log-rank test. For multivariate analysis, p values were calculated using the Cox proportional hazard model.

FIG. 23 provides IC50 values for recombinant single and cis PI3K mutant proteins for the PI3Kα inhibitors alpelisib and GDC-0077. Data are averages (n=3).

FIG. 24 provides PIK3CA exon coverage by ctDNA testing. Exons are numbered based on historical nomenclature and RefSeq (O'Leary et al., Nucleic Acids Res 44, D733-745 (2016)). Amino acids encoded by exons, and the mutations tested in this study are denoted. Exons sequenced by the Foundation Medicine Foundation One Liquid test are highlighted in blue.

### 5. DETAILED DESCRIPTION OF THE INVENTION

The present disclosure relates to methods for determining the responsiveness of a cancer cell or a subject suffering from cancer to a PI3K inhibitor and kits relating thereto. The present disclosure also relates to methods for treating a subject having a cancer (e.g., breast cancer), where the subject has been determined to be likely to respond to a PI3K inhibitor. In particular, the present disclosure provides use of two or more PI3KCA mutations for determining the responsiveness of a cancer cell or a subject suffering from cancer to a PI3K inhibitor.

For purposes of clarity of disclosure and not by way of limitation, the detailed description is divided into the following subsections:

5.1 Definitions;

5.2 PIK3CA Mutations;

5.3 Methods of Treatment;

5.4 Cancer Targets;

5.5 Detection of PIK3CA Mutations; and

5.6 Kits.

### 5.1 Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

A “biological sample” or “sample,” as used interchangeably herein, refers to a sample of biological material obtained from a subject, including a biological fluid and/or body fluid, e.g., blood, plasma, serum, stool, urine, lymphatic fluid, ascites, ductal lavage, nipple aspirate, saliva, broncho-alveolar lavage, tears and cerebrospinal fluid. In certain non-limiting embodiments, the presence of one or more biomarkers of the present disclosure are determined in one or more samples obtained from a subject, e.g., plasma samples. In certain embodiments, the sample contains nucleic acids, e.g., DNA, that are is released into vascular system, present in circulation, e.g., blood or plasma, present in body fluid, e.g., plasma, serum, urine or pleural effusion or is extracellular, e.g., outside of (not located within) any cell, bound or unbound to the cell surface.

As used herein, the term “disease” refers to any condition or disorder that damages or interferes with the normal function of a cell, tissue, or organ.

An “effective amount” of a substance as that term is used herein is that amount sufficient to effect beneficial or desired results, including clinical results, and, as such, an “effective amount” depends upon the context in which it is being applied. An effective amount can be administered in one or more administrations.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more sign or symptoms, diminishment of extent of disease, stabilized (i.e., not worsening) state of disease, prevention of disease, delay or slowing of disease progression, and/or amelioration or palliation of the disease state. The decrease can be an about 10%, about 20%, about 30%, about 40%, about 50%, about 60%, about 70%, about 80%, about 90%, about 95%, about 98% or about 99% decrease in severity of complications or symptoms. “Treatment” can also mean prolonging survival as compared to expected survival if not receiving treatment.

An “anti-cancer agent,” as used herein, can be any molecule, compound, chemical or composition that has an anti-cancer effect.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

### 5.2. PIK3CA Mutations

Two or more PIK3CA mutations can be used for determining the responsiveness of a cancer cell or a subject suffering from cancer to a PI3K inhibitor.

In certain embodiments, the PIK3CA mutation is an insertions, deletions or substitutions relative to a reference PIK3CA gene described below. Such insertions, deletions or substitutions may result in a nonsense mutation, a frameshift mutation, a missense mutation or a termination relative to the reference PIK3CA gene and/or protein. In certain embodiments, the PIK3CA mutation is a substitution.

A “reference,” “reference control” or “control,” as used interchangeably herein, can be a human PIK3CA nucleic acid having the sequence as set forth in NCBI database accession no. NG_012113.2, or a nucleic acid encoding a PIK3CA protein molecule that has the amino acid sequence set forth in NCBI database accession no. GI:126302584.

Reference PIK3CA nucleic acids for non-human species are known or can be determined according to methods known in the art, for example, where the sequence is the allele represented in the majority of the population of that species.

In certain embodiments, the two or more PIK3CA mutations used in the presently disclosed methods are selected from Tables 4 and 5 disclosed herein.

In certain embodiments, the two or more PIK3CA mutations are selected from Tables 4 and 5 disclosed herein.

In certain embodiments, the two or more PIK3CA mutations comprise a first PIK3CA mutation and a second PIK3CA mutation.

In certain embodiments, the first PIK3CA mutation is selected from Tables 4 and 5. In certain embodiments, the first PIK3CA mutation is selected from the group consisting of E542, E545, and H1047. In certain embodiments, the first PIK3CA mutation is selected from the group consisting of E542K, E545K, and H1047R.

In certain embodiments, the second PIK3CA mutation is selected from Tables 4 and 5. In certain embodiments, the second PIK3CA mutation is selected from the group consisting of E453, E726, and M1043. In certain embodiments, the second PIK3CA mutation is selected from the group consisting of E453Q, E453K, E726K, M1043I, and M1043L.

In certain embodiments, the two or more PIK3CA mutations comprise a first PIK3CA mutation H1047R and a second PIK3CA mutation E453Q. In certain embodiments, the two or more PIK3CA mutations comprise a first PIK3CA mutation H1047R and a second PIK3CA mutation E453K. In certain embodiments, the two or more PIK3CA mutations comprise a first PIK3CA mutation H1047R and a second PIK3CA mutation E726K. In certain embodiments, the two or more PIK3CA mutations comprise a first PIK3CA mutation E545K and a second PIK3CA mutation E726K. In certain embodiments, the two or more PIK3CA mutations comprise a first PIK3CA mutation E545K and a second PIK3CA mutation M1043L. In certain embodiments, the two or more PIK3CA mutations comprise a first PIK3CA mutation E545K and a second PIK3CA mutation M1043I. In certain embodiments, the two or more PIK3CA mutations comprise a first PIK3CA mutation E545K and a second PIK3CA mutation E453Q. In certain embodiments, the two or more PIK3CA mutations comprise a first PIK3CA mutation E545K and a second PIK3CA mutation E453K. In certain embodiments, the two or more PIK3CA mutations comprise a first PIK3CA mutation E542K and a second PIK3CA mutation E726K. In certain embodiments, the two or more PIK3CA mutations comprise a first PIK3CA mutation E542K and a second PIK3CA mutation M1043L. In certain embodiments, the two or more PIK3CA mutations comprise a first PIK3CA mutation E542K and a second PIK3CA mutation M1043I. In certain embodiments, the two or more PIK3CA mutations comprise a first PIK3CA mutation E542K and a second PIK3CA mutation E453Q. In certain embodiments, the two or more PIK3CA mutations comprise a first PIK3CA mutation E542K and a second PIK3CA mutation E453K.

### 5.3. Methods of Treatment

The two or more PIK3CA mutations can be used to predict the responsiveness of a cancer cell or a subject suffering from cancer to a PI3K inhibitor. Thus, the present disclosure provides methods for determining the responsiveness of a cancer cell or a subject suffering from cancer to a PI3K inhibitor. In certain embodiments, the method comprises determining the presence of two or more PIK3CA mutations in a sample (e.g., a biological sample) from a subject (e.g., a subject suffering from cancer), wherein the presence of the two or more PIK3CA mutations indicates that the subject is likely to be responsive to a PI3K inhibitor.

Furthermore, the present disclosure provides methods for treating a subject having a cancer. In certain embodiments, the method comprises (a) identifying a subject as likely to be responsive to a PI3K inhibitor by the above method, and (b) administering a therapeutically effective amount of a PI3K inhibitor to the subject identified in (a).

In certain embodiments, the two or more PIK3CA mutations are selected from those disclosed in Section 5.2.

In certain embodiments, a subject having detectable levels of the two or more PIK3CA mutations has a prolonged response to a PI3K inhibitor than a subject having no detectable levels of the two or more PIK3CA mutations. In certain embodiments, a subject having the two or more PIK3CA mutations has a longer progression-free survival (PFS) than a subject having no detectable levels of the two or more PIK3CA mutations. In certain embodiments, the PI3K inhibitor prolongs the survival of a subject having the two or more PIK3CA mutations for about 1 month, about 2 months, about 3 months, about 6 months, about 1 year, about 2 years, about 3 years, about 4 years, about 5 years, about 10 years or more, longer than a subject having no detectable levels of the two or more PIK3CA mutations.

In certain embodiments, the PI3K inhibitor reduces the growth of a tumor in a subject having detectable levels of the two or more PIK3CA mutations more than a subject having no detectable levels of the two or more PIK3CA mutations. In certain embodiments, the PI3K inhibitor reduces the size of a tumor in a subject having detectable levels of the two or more PIK3CA mutations more than a subject having no detectable levels of the two or more PIK3CA mutations. In certain embodiments, the PI3K inhibitor reduces the weight of a tumor in a subject having detectable levels of the two or more PIK3CA mutations more than a subject having no detectable levels of the two or more PIK3CA mutations. In certain embodiments, the PI3K inhibitor inhibits the metastasis of a tumor in a subject having detectable levels of the two or more PIK3CA mutations more than a subject having no detectable levels of the two or more PIK3CA mutations.

Non-limiting examples of PI3K inhibitors include compounds, molecules, chemicals, polypeptides and proteins that inhibit and/or reduce the expression and/or activity of PI3K. In certain embodiments, the PI3K inhibitor is an ATP-competitive inhibitor of PI3K. In certain embodiments, the PI3K inhibitor is a PI3Kα inhibitor. In certain embodiments, the PI3K inhibitor is derived from imidazopyridine or 2-aminothiazole compounds. In certain embodiments, the PI3K inhibitor is selected from the group consisting of BYL719, INK-1114, INK-1117, NVP-BYL719, SRX2523, LY294002, PIK-75, PKI-587, A66, CH5132799, GDC-0032 (taselisib), GDC-0077, and combinations thereof. In certain embodiments, the PI3K inhibitor is BYL719. In certain embodiments, the PI3K inhibitor is GDC-0032.

Further, the PI3K inhibitors are those disclosed in Schmidt-Kittler et al., Oncotarget (2010) 1(5):339-348; Wu et al., Med. Chem. Comm. (2012) 3:659-662; Hayakawa et al., Bioorg. Med. Chem. (2007) 15(17): 5837-5844; and PCT Patent Publication Nos. WO2013/049581 and WO2012/052745, the contents of which are herein incorporated by reference in their entireties.

Furthermore, the PI3K inhibitors can include ribozymes, antisense oligonucleotides, shRNA molecules and siRNA molecules that specifically inhibit and/or reduce the expression or activity of PI3K. In certain embodiments, the PI3K inhibitor comprises an antisense, shRNA, or siRNA nucleic acid sequence homologous to at least a portion of a PI3K nucleic acid sequence, e.g., the nucleic acid sequence of a PI3K alpha subunit such as PIK3CA, wherein the homology of the portion relative to the PI3K sequence is at least about 75 or at least about 80 or at least about 85 or at least about 90 or at least about 95 or at least about 98 percent, where percent homology can be determined by, for example, BLAST or FASTA software. In certain non-limiting embodiments, the complementary portion constitutes at least 10 nucleotides or at least 15 nucleotides or at least 20 nucleotides or at least 25 nucleotides or at least 30 nucleotides and the antisense nucleic acid, shRNA or siRNA molecules may be up to 15 or up to 20 or up to 25 or up to 30 or up to 35 or up to 40 or up to 45 or up to 50 or up to 75 or up to 100 nucleotides in length. Antisense, shRNA, or siRNA molecules may comprise DNA or atypical or non-naturally occurring residues, for example, but not limited to, phosphorothioate residues.

In certain embodiments, the PI3K inhibitor can be used alone or in combination with one or more anti-cancer agents. Non-limiting examples of anti-cancer agents include chemotherapeutic agents, radiotherapeutic agents, cytokines, anti-angiogenic agents, apoptosis-inducing agents, anti-cancer antibodies, anti-cyclin-dependent kinase agents, and/or agents which promote the activity of the immune system including but not limited to cytokines such as but not limited to interleukin 2, interferon, anti-CTLA4 antibody, anti-PD-1 antibody, and/or anti-PD-Ll antibody. For example, but not by way of limitation, a PI3K inhibitor can be used in combination with letrozole or exemestane. In certain embodiments, the PI3K inhibitor and the one or more anti-cancer agents are administered to a subject as part of a treatment regimen or plan. In certain embodiments, the PI3K inhibitor and one or more anti-cancer agents are not physically combined prior to administration. In certain embodiments, the PI3K inhibitor and one or more anti-cancer agents are not administered over the same time frame.

### 5.4. Cancer Targets

Non-limiting examples of cancers that may be subject to the presently disclosed subject matter include biliary tree cancer, hepatocellular carcinoma, cancers of the head and neck, gastric cancer, endometrial carcinoma, breast cancer, brain cancer, colorectal cancer, uterine cancer, bladder cancer, lung cancer, liver cancer, glioma, head and neck cancers, stomach cancer, cervical cancer, prostate cancer, prostate adenoma, melanoma, cutaneous melanoma, upper tract urothelial cancers, esophageal cancer, esophageal squamous cell carcinoma, esophageal adenocarcinoma, cutaneous squamous cell cancers, rectal cancer, rectal adenoma, ampullary cancer, cancer of unknown primary, oropharynx squamous cell cancer, intrahepatic cholangiocarcinoma, cholangiocarcinoma, esophagogastric adenocarcinoma, mucinous carcinoma, anaplastic astrocytoma, astrocytoma, kidney cancer, papillary renal cell carcinoma, ovarian cancer, high-grade serous ovarian cancer, poorly differentiated thyroid cancer, thyroid cancer, nasopharyngeal cancer, medulloblastoma, salivary duct cancer, non-seminomatous germ cell tumor, basaloid penile squamous cell cancer, and penile cancer. In certain embodiment, the cancer is a breast cancer. In certain embodiments, the cancer is an estrogen-receptor positive metastatic breast cancer.

### 5.5. Detection of PIK3CA Mutations

In certain embodiments, the two or more PIK3CA mutations disclosed herein can be detected in cell free nucleic acids isolated from biological samples obtained from a subject, such as a plasma sample, or other biological fluid, as described above. In certain embodiments, the cell free nucleic acids comprise circulating tumor DNA (ctDNA).

There are several platforms that are known in the art and currently available to isolate cell free nucleic acids from biological samples. In certain embodiments, isolation of DNA from a biological sample is based on extraction methods using organic solvents such as a mixture of phenol and chloroform, followed by precipitation with ethanol (see, for example, J. Sambrook et al., “Molecular Cloning: A Laboratory Manual”, 1989, 2nd Ed., Cold Spring Harbour Laboratory Press: New York, N.Y.). Additional non-limiting examples include salting out DNA extraction (see, for example, P. Sunnucks et al., Genetics, 1996, 144: 747-756; and S. M. Aljanabi and I. Martinez, Nucl. Acids Res. 1997, 25: 4692-4693), the trimethylannnonium bromide salts DNA extraction method (see, for example, S. Gustincich et al., BioTechniques, 1991, 11: 298-302) and the guanidinium thiocyanate DNA extraction method (see, for example, J. B. W. Hammond et al., Biochemistry, 1996, 240: 298-300).

Non-limiting examples of kits that can be used to extract DNA from bodily fluids include kits that are commercially available from, for example, BD Biosciences Clontech (Palo Alto, Calif.), Epicentre Technologies (Madison, Wis.), Gentra Systems, Inc. (Minneapolis, Minn.), MicroProbe Corp. (Bothell, Wash.), Organon Teknika (Durham, N.C.), and Qiagen Inc. (Valencia, Calif.). Sensitivity, processing time and cost may be different from one kit to another. One of ordinary skill in the art can easily select the kit(s) most appropriate for the particular sample to be analyzed.

The presently disclosure further provides methods for detecting and/or determining the presence of two or more PIK3CA mutations. For example, but not by way of limitation, such methods include polymerase chain reaction (PCR), including, but not limited to, real-time PCR, quantitative PCR, fluorescent PCR, RT-MSP (RT methylation specific polymerase chain reaction and digital PCR, in situ hybridization, fluorescent in situ hybridization (“FISH”), gel electrophoresis, radioimmunoassay, direct radio-labeling of DNA, sequencing and sequence analysis, single-molecule sequencing, SMRTbell sequencing, Sanger sequencing, microarray analysis and other techniques known in the art.

In certain embodiments, a PIK3CA mutation can be detected through the use of DROPLET DIGITAL™ PCR (ddPCR™), which is a method for performing digital PCR based on water-oil emulsion droplet technology. Alternatively or additionally, the PIK3CA mutations disclosed herein can be detected through direct plasma sequencing by means of tagged-amplicon deep sequencing (see, for example, Forshew et al., Sci. Transl. Med. (2012) 4:136, p. 136).

In certain embodiments, the two or more PIK3CA mutations are determined by sequencing, e.g., next generation sequencing. In certain embodiments, the two or more PIK3CA mutations are determined using a microarray. In certain embodiments, the two or more PIK3CA mutations are determined using an assay that comprises an amplification reaction, such as a polymerase chain reaction (PCR).

### 5.6. Kits

The present disclosure also provides kits for determining the responsiveness of a cancer cell or a subject suffering from a cancer to a PI3K inhibitor. In certain embodiments, the kit comprises a means for detecting two or more PIK3CA mutations set forth in Section 5.2 herein.

Types of kits include, but are not limited to, packaged biomarker-specific probe and primer sets (e.g., TaqMan probe/primer sets), arrays/microarrays, which further contain one or more probes, primers, biomarker-specific beads or other reagents for detecting one or more biomarkers of the present invention.

In certain embodiment, the kit comprises a pair of oligonucleotide primers, suitable for polymerase chain reaction (PCR) or nucleic acid sequencing, for detecting the PIK3CA mutations. A pair of primers may comprise nucleotide sequences complementary to a PIK3CA mutation set forth above, and be of sufficient length to selectively hybridize with said biomarker. Alternatively, the complementary nucleotides may selectively hybridize to a specific region in close enough proximity 5′ and/or 3′ to the PIK3CA mutation position to perform PCR and/or sequencing. Multiple specific primers may be included in the kit to simultaneously assay large number of PIK3CA mutations s.

The kit may also comprise one or more polymerases, reverse transcriptase, and nucleotide bases, wherein the nucleotide bases can be further detectably labeled. For example, in certain embodiments, the kits may comprise containers (including microliter plates suitable for use in an automated implementation of the method), each with one or more of the various reagents (typically in concentrated form) utilized in the methods, including, for example, pre-fabricated microarrays, buffers, the appropriate nucleotide triphosphates (e.g., dATP, dCTP, dGTP and dTTP, or rATP, rCTP, rGTP and UTP), reverse transcriptase, DNA polymerase, RNA polymerase, and one or more probes and primers of the present disclosure (e.g., appropriate length poly(T) or random primers linked to a promoter reactive with the RNA polymerase).

In certain embodiments, a primer may be at least about 10 nucleotides or at least about 15 nucleotides or at least about 20 nucleotides in length and/or up to about 200 nucleotides or up to about 150 nucleotides or up to about 100 nucleotides or up to about 75 nucleotides or up to about 50 nucleotides in length.

In certain embodiment, the oligonucleotide primers may be immobilized on a solid surface or support, for example, on a microarray, wherein the position of each oligonucleotide primer bound to the solid surface or support is known and identifiable. The terms “arrays,” “microarrays,” and “DNA chips” are used herein interchangeably to refer to an array of distinct polynucleotides affixed to a substrate, such as glass, plastic, paper, nylon or other type of membrane, filter, chip, bead, or any other suitable solid support. The polynucleotides can be synthesized directly on the substrate, or synthesized separate from the substrate and then affixed to the substrate. The arrays are prepared using known methods.

In certain embodiments, the kit comprises at least one nucleic acid probe, suitable for in situ hybridization or fluorescent in situ hybridization, for detecting the PIK3CA mutations. Such kits will generally comprise one or more oligonucleotide probes that have specificity for various PIK3CA mutations. Means for testing multiple PIK3CA mutations may optionally be comprised in a single kit.

In certain embodiment, the kit comprises one or more pairs of primers, probes or microarrays suitable for detecting two or more PIK3CA mutations. In certain embodiments, the two or more PIK3CA mutations are selected from Tables 4 and 5. In certain embodiments, the two or more PIK3CA mutations comprise a first PIK3CA mutation and a second PIK3CA mutation. In certain embodiments, the first PIK3CA mutation is selected from Tables 4 and 5. In certain embodiments, the first PIK3CA mutation is selected from the group consisting of E542, E545, and H1047. In certain embodiments, the first PIK3CA mutation is selected from the group consisting of E542K, E545K, and H1047R. In certain embodiments, the second PIK3CA mutation is selected from Tables 4 and 5. In certain embodiments, the second PIK3CA mutation is selected from the group consisting of E453, E726, and M1043. In certain embodiments, the second PIK3CA mutation is selected from the group consisting of E453Q, E453K, E726K, M1043I, and M1043L.

In certain embodiment, the kit comprises one or more pairs of primers, probes or microarrays suitable for detecting PIK3CA mutations E542K, E545K, H1047R, E453Q, E453K, E726K, M1043I, and M1043L. In certain embodiments, the kit comprises one or more pairs of primers, probes or microarrays suitable for detecting two or more PIK3CA mutations comprising a first PIK3CA mutation H1047R and a second PIK3CA mutation E453Q. In certain embodiments, the kit comprises one or more pairs of primers, probes or microarrays suitable for detecting two or more PIK3CA mutations comprising a first PIK3CA mutation H1047R and a second PIK3CA mutation E453K. In certain embodiments, the kit comprises one or more pairs of primers, probes or microarrays suitable for detecting two or more PIK3CA mutations comprising a first PIK3CA mutation H1047R and a second PIK3CA mutation E726K. In certain embodiments, the kit comprises one or more pairs of primers, probes or microarrays suitable for detecting two or more PIK3CA mutations comprising a first PIK3CA mutation E545K and a second PIK3CA mutation E726K. In certain embodiments, the kit comprises one or more pairs of primers, probes or microarrays suitable for detecting two or more PIK3CA mutations comprising a first PIK3CA mutation E545K and a second PIK3CA mutation M1043L. In certain embodiments, the kit comprises one or more pairs of primers, probes or microarrays suitable for detecting two or more PIK3CA mutations comprising a first PIK3CA mutation E545K and a second PIK3CA mutation M1043I. In certain embodiments, the kit comprises one or more pairs of primers, probes or microarrays suitable for detecting two or more PIK3CA mutations comprising a first PIK3CA mutation E545K and a second PIK3CA mutation E453Q. In certain embodiments, the kit comprises one or more pairs of primers, probes or microarrays suitable for detecting two or more PIK3CA mutations comprising a first PIK3CA mutation E545K and a second PIK3CA mutation E453K. In certain embodiments, the kit comprises one or more pairs of primers, probes or microarrays suitable for detecting two or more PIK3CA mutations comprising a first PIK3CA mutation E542K and a second PIK3CA mutation E726K. In certain embodiments, the kit comprises one or more pairs of primers, probes or microarrays suitable for detecting two or more PIK3CA mutations comprising a first PIK3CA mutation E542K and a second PIK3CA mutation M1043L. In certain embodiments, the kit comprises one or more pairs of primers, probes or microarrays suitable for detecting two or more PIK3CA mutations comprising a first PIK3CA mutation E542K and a second PIK3CA mutation M1043I. In certain embodiments, the kit comprises one or more pairs of primers, probes or microarrays suitable for detecting two or more PIK3CA mutations comprising a first PIK3CA mutation E542K and a second PIK3CA mutation E453Q. In certain embodiments, the kit comprises one or more pairs of primers, probes or microarrays suitable for detecting two or more PIK3CA mutations comprising a first PIK3CA mutation E542K and a second PIK3CA mutation E453K.

Any suitable primers or probes known in the art can be used with the present disclosure. Non-limiting examples of primers for detecting PIK3CA mutations are disclosed in Examples 2 and 3 herein.

In certain non-limiting embodiments, the measurement means in the kit employs an array, the two or more PIK3CA mutations set forth above constitutes at least 10 percent or at least 20 percent or at least 30 percent or at least 40 percent or at least 50 percent or at least 60 percent or at least 70 percent or at least 80 percent of the species of markers represented on the microarray.

In certain embodiments, the kit comprises one or more probes, primers, microarrays/arrays, beads, detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, and the like) to detect the presence of a reference control. Non-limiting examples of a reference control are described above in Section 5.2.

In certain embodiments, the kit further includes instructions for using the kit to detect the PIK3CA mutations of interest. For example, the instructions can describe that the presence of at least two PIK3CA mutation indicates a subject suffering from a cancer is responsive to a PI3K inhibitor.

### 6. EXAMPLE

The presently disclosed subject matter will be better understood by reference to the following Example, which is provided as exemplary of the presently disclosed subject matter, and not by way of limitation.

### Example 1: Compound PIK3CA Mutations in PI3K Activation and Response to PI3K Inhibition

PIK3CA mutations represent the most frequent oncogenic driver lesions found in ER+ metastatic breast cancers (ER+MBC). While single PIK3CA mutations function as oncogenes, patients possessing these mutations alone are likely to derive marginal clinical benefit from PI3K inhibitors, suggesting that additional genomic factors cooperate with single PIK3CA mutations to yield better response to PI3K inhibitor therapy. The inventor sequenced the largest known cohort of breast cancers (n=1918) and discovered dual PIK3CA mutations in ˜15% of ER+PIK3CA-mutant breast cancers. Dual PIK3CA mutations are clonal, occur more frequently in ER+/HER2-breast cancer, and are found in both therapy naïve and metastatic tumors. Dual PIK3CA mutations are most frequently found at specific minor hotspot amino acid positions (E453Q, E726K, M1043L) combined with major hotspot positions (E545K, H1047R). Dual PIK3CA mutations are also compound mutations in cell lines and patient samples, in cis on the same allele. The inventor's preliminary data demonstrate that dual PIK3CA mutations are in cis (i.e. on the same allele, resulting in a protein with two mutations). Dual PIK3CA mutations increase kinase activity and PI3K pathway signaling and result in greater efficacy of cellular response to PI3K inhibition as compared to single PIK3CA mutations. The inventor's findings suggest a novel model of mutational dosage for oncogene activation for PIK3CA and sensitivity to targeted therapy.

### Objectives

To determine the effects of compound PIK3CA mutations on normal cell signaling and growth in vitro and in vivo. To elucidate the biochemical effects of compound PIK3CA mutations on lipid binding. To measure the effects of PI3Kα inhibitors on compound PIK3CA mutant cell growth

### Methods

PIK3CA mutant overexpression into MCF10A (normal breast epithelial cells) and NIH-3T3 (normal mouse fibroblasts) cell lines. Crystal violet assays were used to measure cell growth and proliferation. Western blotting was used to measure PI3K pathway signaling. Liposome binding assays were used to recombinant PI3K protein complexes. Murine xenograft implantation was used to model tumor growth in vivo.

### Results

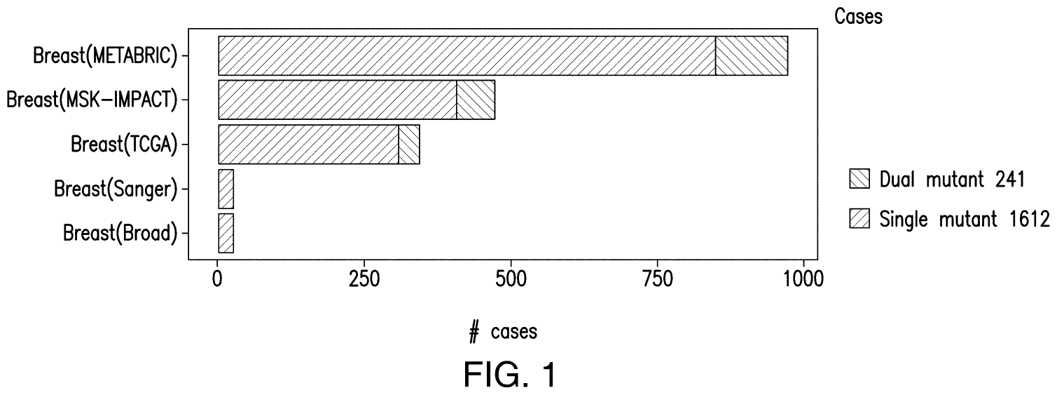

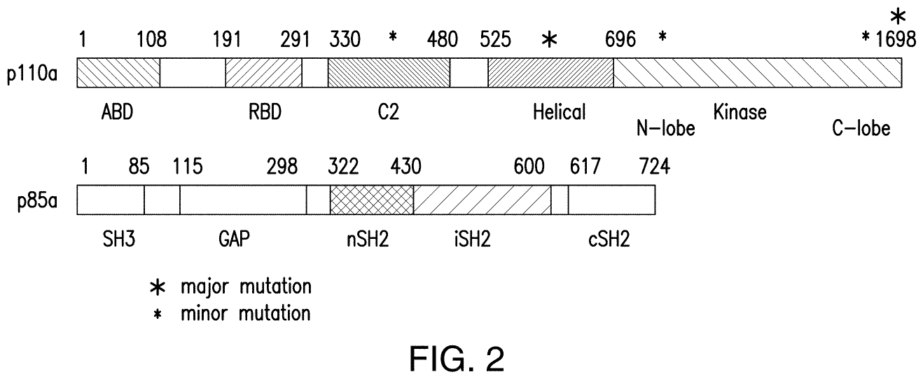

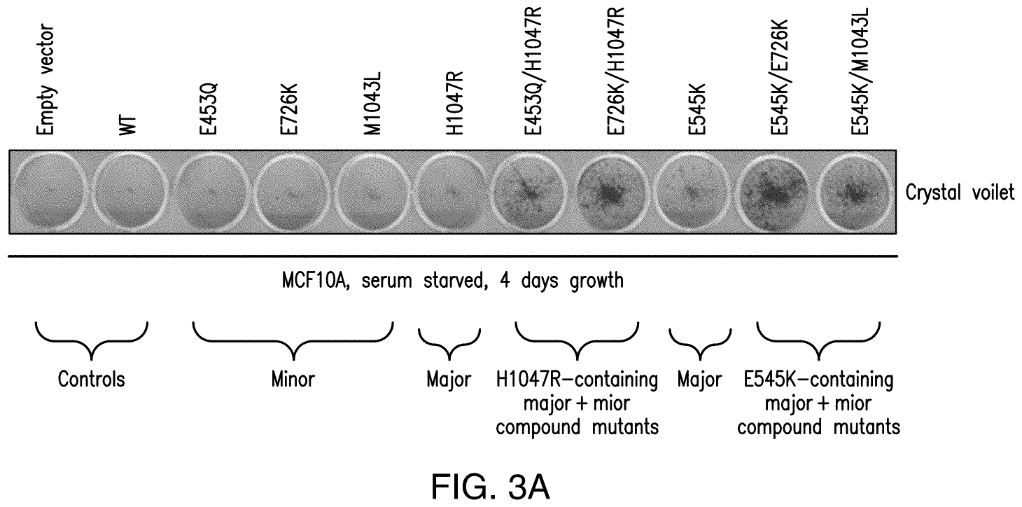

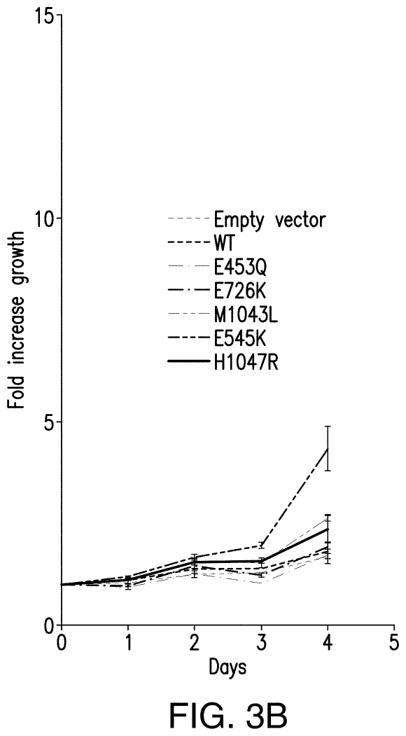

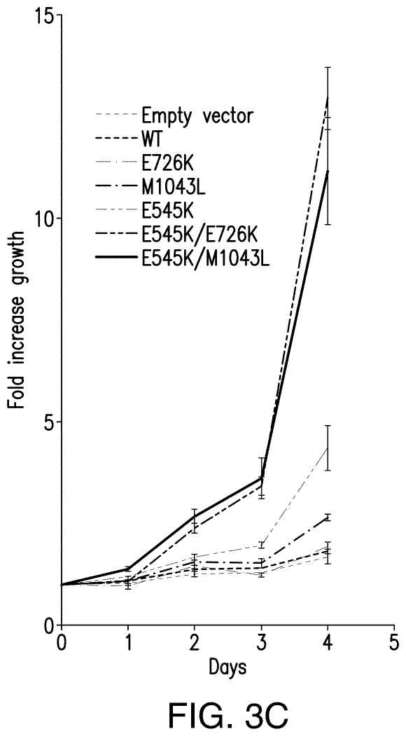

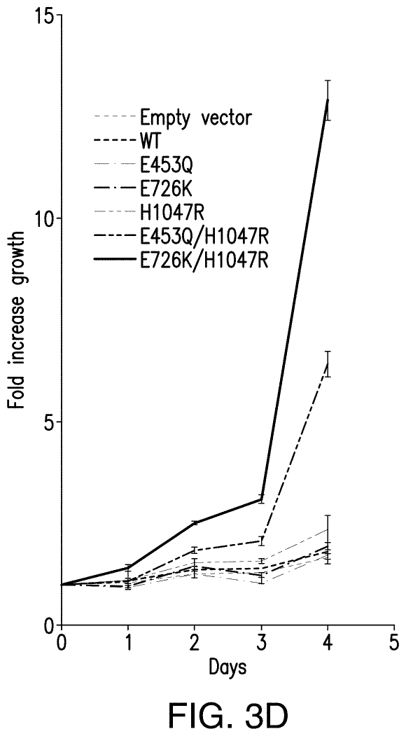

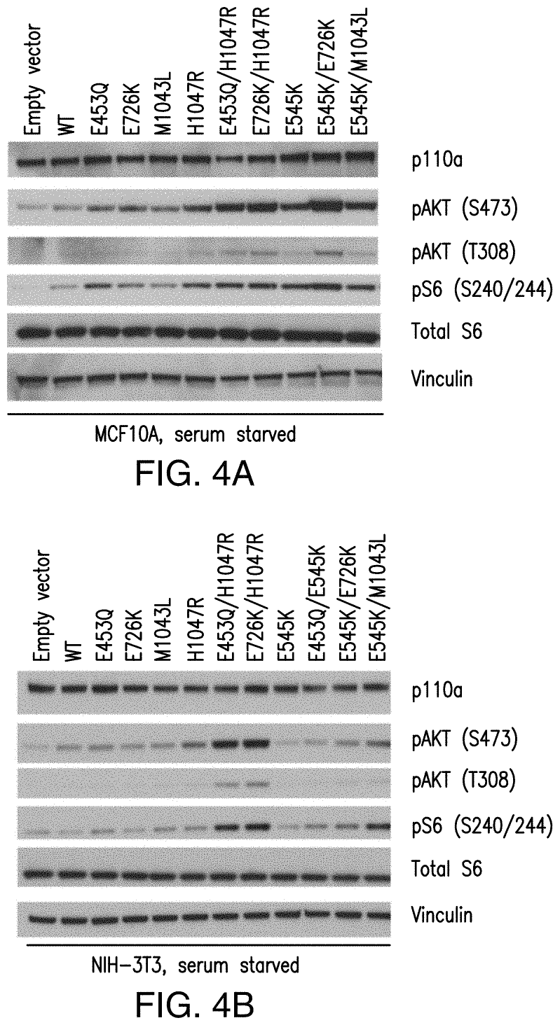

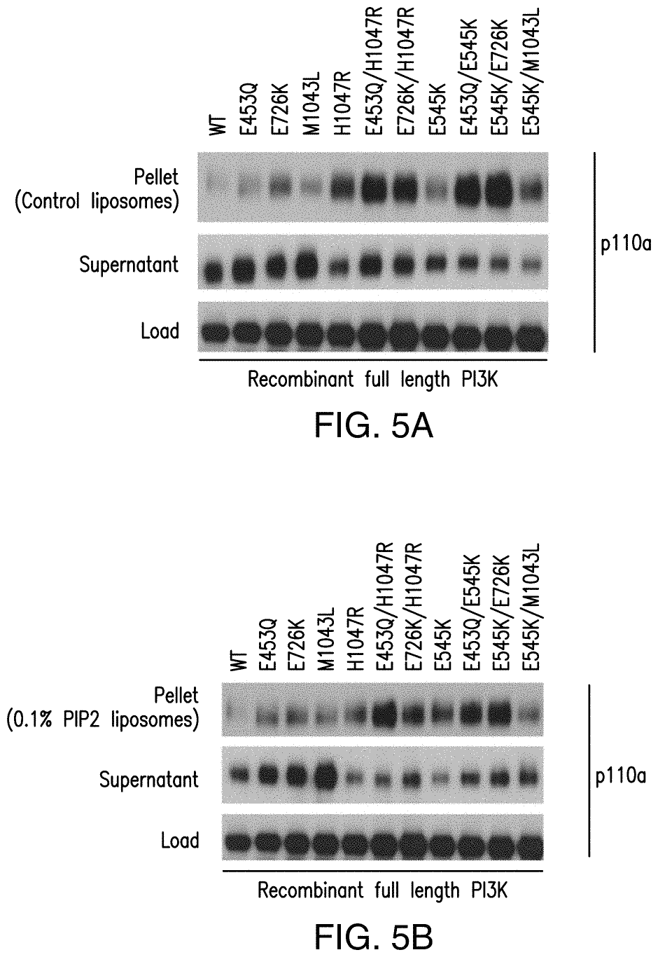

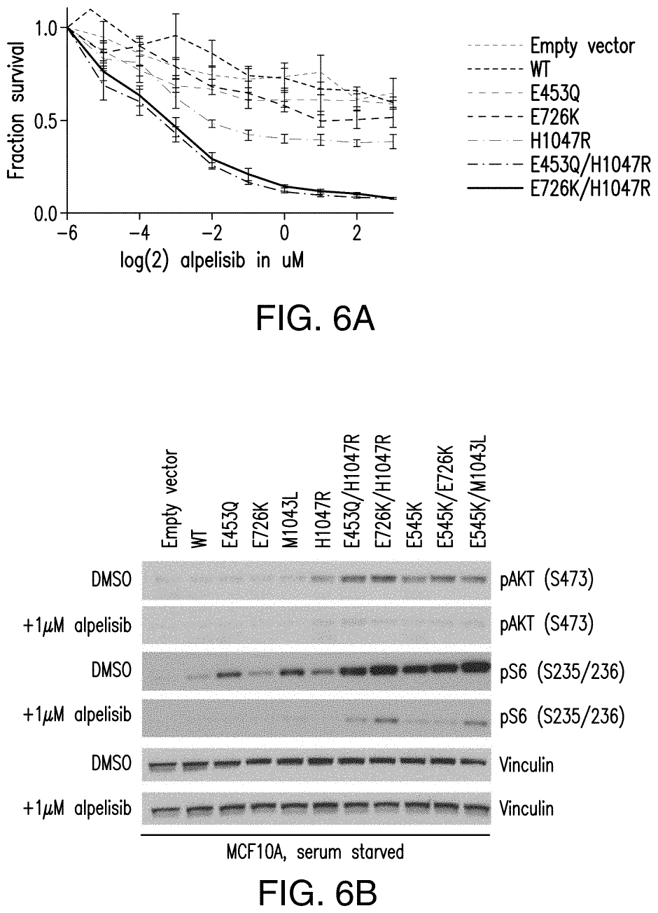

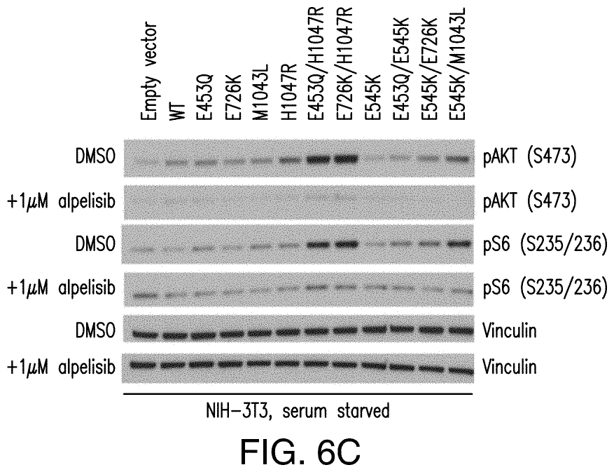

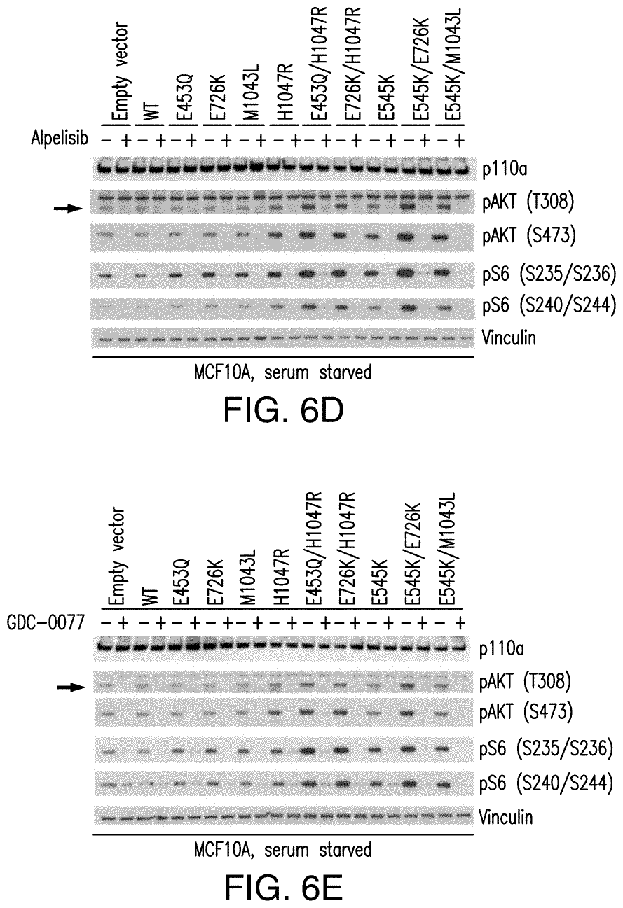

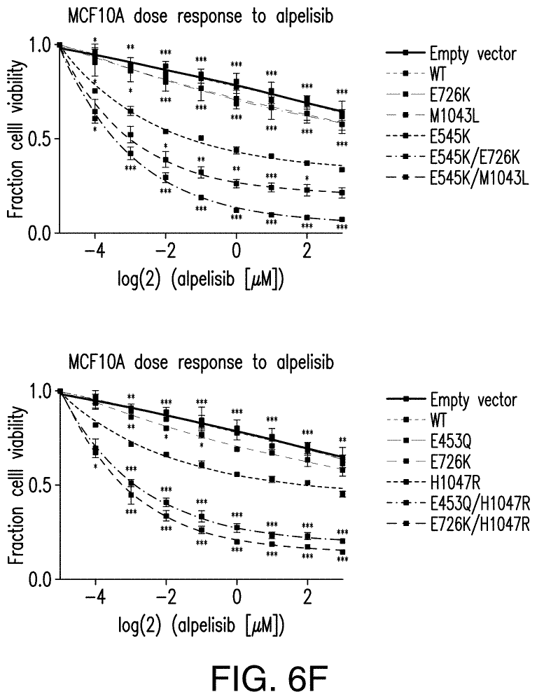

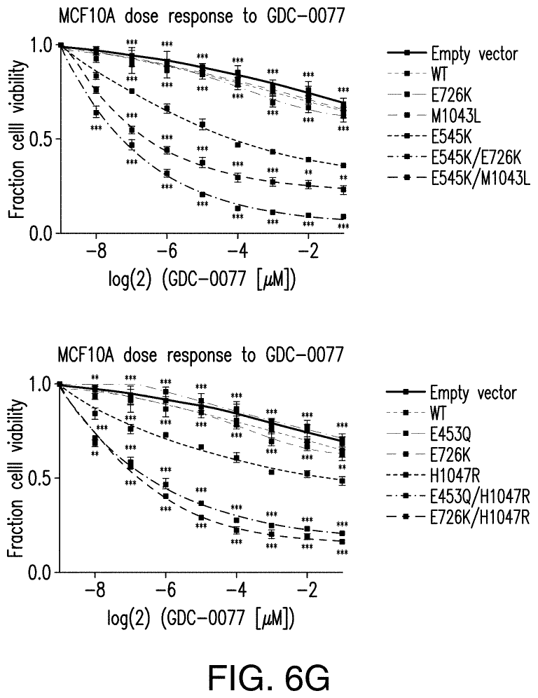

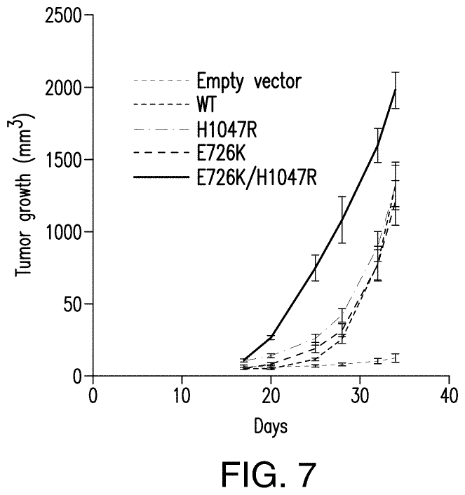

The distribution of PIK3CA mutation type in multiple breast cancer datasets (n=1853) is shown in FIG. 1. The incidence of dual PIK3CA mutants across PIK3CA mutated breast cancer is ˜15%. The domain schematic of p110α protein and positions of major and minor mutations as asterisks is shown in FIG. 2. Results are shown in FIGS. 3-7. The effects of PIK3CA mutations on cell growth were shown in FIGS. 3A-3D. The effect of PIK3CA mutations on cell signaling were shown in FIGS. 4A and 4B. The effect of the PIK3CA mutation on activity of PI3K complexes were shown in FIGS. 5A and 5B. The effect of PI3K inhibitors on cell survival and cell signaling were shown in FIGS. 6A-6C. The growth of PIK3CA-mutant and wild type and empty vector control NIH-3T3 cells in murine xenografts was shown in FIG. 7.

### Conclusions

The data demonstrate that compound PIK3CA mutations increase cell growth in vitro and in vivo in a PI3K-pathway dependent manner, more than single hotspot mutants. One biochemical mechanism of increased activity of compound PIK3CA mutants is through more avid binding to both uncharged lipids and PIP2. Compound PIK3CA mutations also increase efficacy of response to the PI3K inhibitor BYL719, with more significant decrease in cell viability than displayed in single mutants.

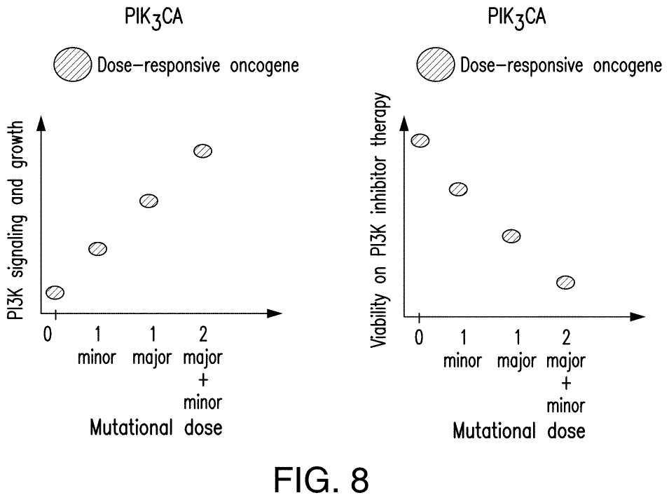

Together, these data support a mutational dosage model for PIK3CA oncogene activation and sensitivity to targeted therapy (FIG. 8).

The consequences of compound PIK3CA mutations in ER+ breast cancer cell lines in vitro and in vivo on PI3K pathway signaling including crosstalk with ER transcription will be investigated. The inventor's recombinant protein complexes will be used to further dissect the mechanism of activation of compound PIK3CA mutations by measuring lipid kinase activity and thermal instability. The inventor's in vitro and in vivo models will be utilized to determine sensitivity to other PI3Kα inhibitors. Together, these preclinical studies form a rationale for a basket clinical trial testing PI3Kα inhibitors in patients with compound PIK3CA mutant tumors.

### Example 2: Double Hit Compound PIK3CA Mutations Enhance Oncogene Activation and Therapeutic Dependency

Activating mutations in PIK3CA, the gene coding for the catalytic subunit (p110 alpha) of phosphoinositide-3-kinase (PI3K) are the most frequent oncogenic alterations across all cancers including in estrogen receptor-positive metastatic breast cancer (ER+MBC). PI3K alpha inhibitors improve survival in ER+MBC, in patients with PIK3CA-mutant tumors. However, there is a wide variation in response even in patients with PIK3CA-mutant tumors and PI3K inhibitors have a narrow therapeutic index with significant on target side effects. Thus, identifying the group of patients who benefit most from PI3K inhibitors is of critical importance. The present example has discovered “double hit” PIK3CA mutations in 10-15% of mutant PIK3CA tumors across all cancers, associated with a major hotspot combined with a recurrent second-site PIK3CA mutation (‘minor mutation’). Double hit PIK3CA mutations are compound, that is in cis on the same allele. Compound PIK3CA mutations increase PI3K activity in recombinant protein, cell, and xenograft models compared to single mutants, through a mechanism combining increased protein complex instability with increased membrane binding. Compound mutations predict for preferential inhibition to PI3Kα inhibitors in vitro and in breast cancer patients. Together, the presently disclosed data support a mutational dosage model for PIK3CA oncogene activation and response to targeted therapy by double hit compound PIK3CA mutations.

PIK3CA is the most frequently mutated oncogene across all human cancers, and codes for p110α, the catalytic subunit of the PI3Kα lipid kinase complex, which is necessary for normal growth and proliferation (Fruman, Cell, 2017, 170, 605-635). p110α binds to the noncatalytic and inhibitory subunit p85α to form PI3Kα. PI3Kα requires multiple inputs for full activation, including binding by membrane-bound receptor tyrosine kinases (RTKs) and Ras, and can be constitutively activated by oncogenic mutations. Single amino acid substitutions in the helical (E542K/E545K) or kinase (H1047R) domains of PI3Kα are the most frequent alterations (‘major hotspots’) (Samuels, Science, 2004, 304, 554) and each of these mutations is considered an oncogenic driver in multiple cancer histologies (Samuels, Cancer Cell, 2005, 7, 561-573; Engelman, Nat Med, 2008, 14, 1351-1356; Isakoff, Cancer Res, 2005, 65, 10992-11000; Zhai, Proc Natl Acad Sci USA, 2005, 102, 18443-18448; Kang, Proc Natl Acad Sci USA, 2005, 102, 802-807).

In breast cancer, PIK3CA mutations are present in 40% of ER+ primary and metastatic tumors (Razavi, Cancer Cell, 2018 34, 427-438) and are predictive for response to PI3K inhibitors (Andre, Journal of Clinical Oncology, 2016, 34, no. 15_suppl.; Baselga, Journal of Clinical Oncology, 2018, 36, no. 18_suppl.).

The potency of PI3K inhibitors is undermined by on target side effects (e.g. hyperglycemia, rash, colitis) which are difficult to manage clinically and result in a narrow therapeutic index. Additionally, loss of PTEN (Jurix, Nature, 2015, 518, 240-244) and relief of negative feedback on the insulin signaling pathway (Hopkins, Nature, 2018, 560, 499-503) are validated mechanisms of resistance to PI3K inhibitors. Therefore, identifying the group of patients with increased sensitivity to PI3K inhibitors is of critical importance. However, beyond single hotspot mutations, additional sensitizing mechanisms remain to elucidated. The present example hypothesized that additional genomic factors cooperate with PIK3CA hotspot mutations to increase their oncogenic phenotype and dependence on the PI3K pathway.

Results

Dual PIK3CA-mutant tumors are frequent across all cancers.

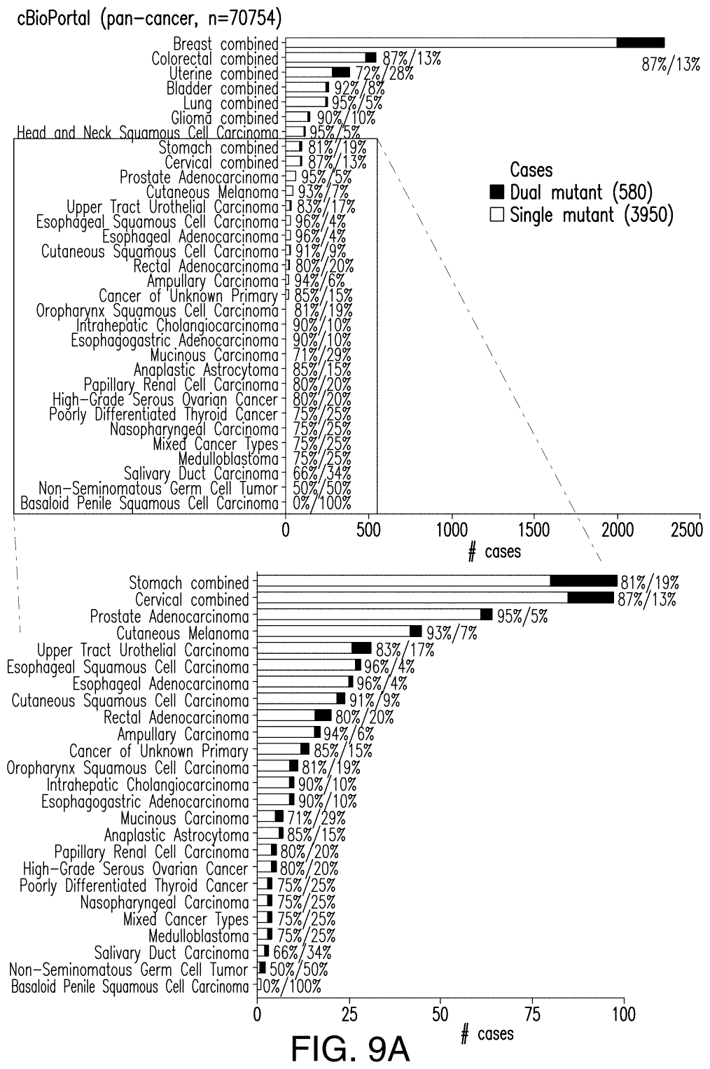

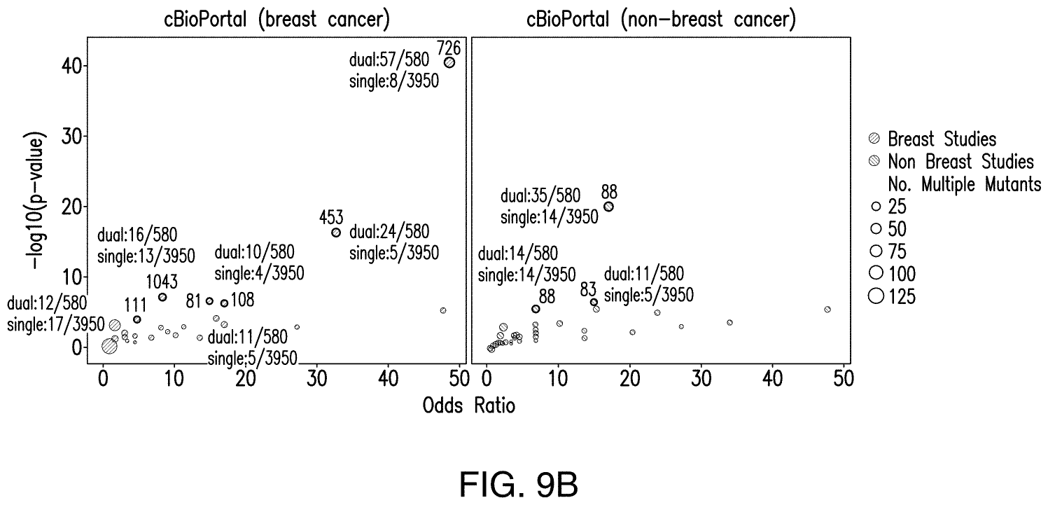

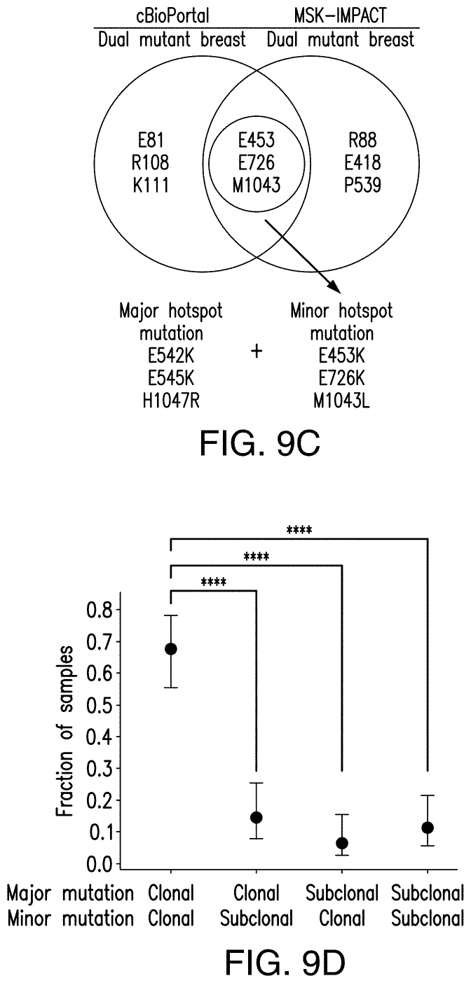

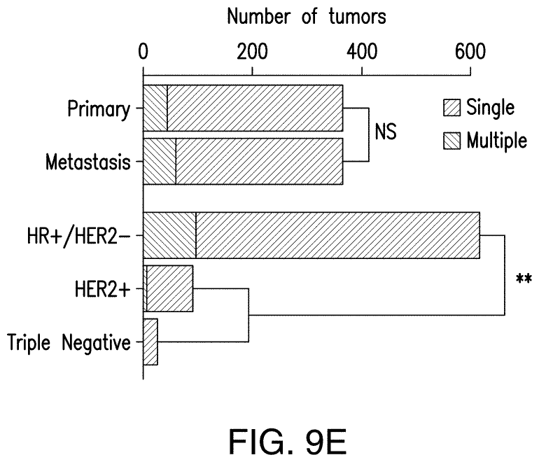

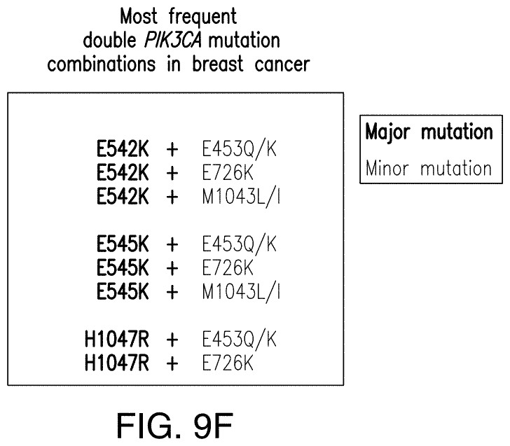

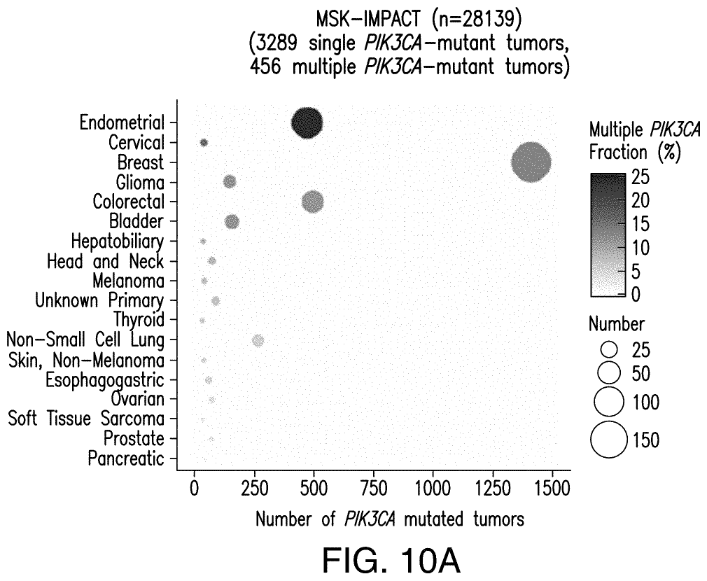

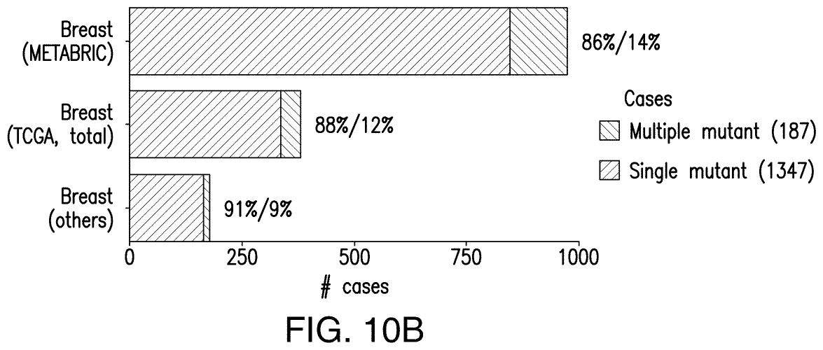

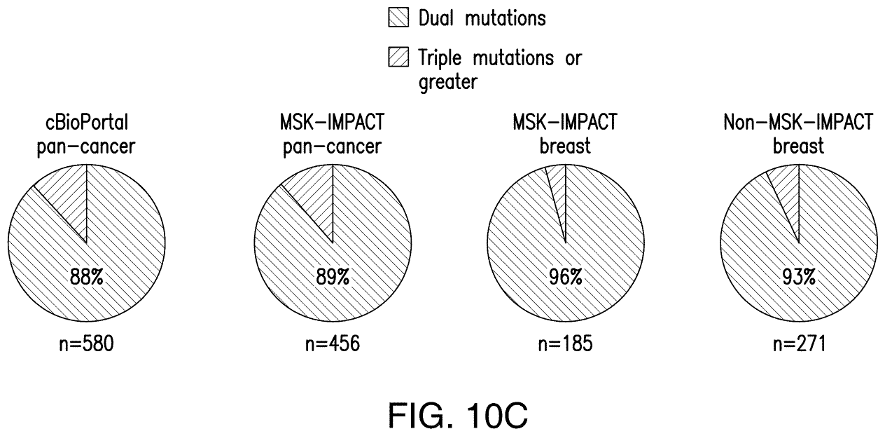

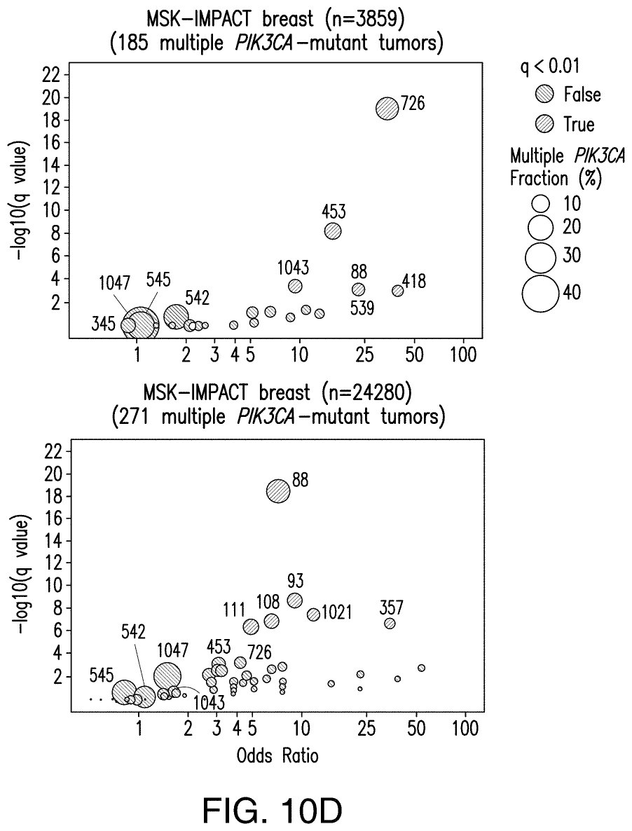

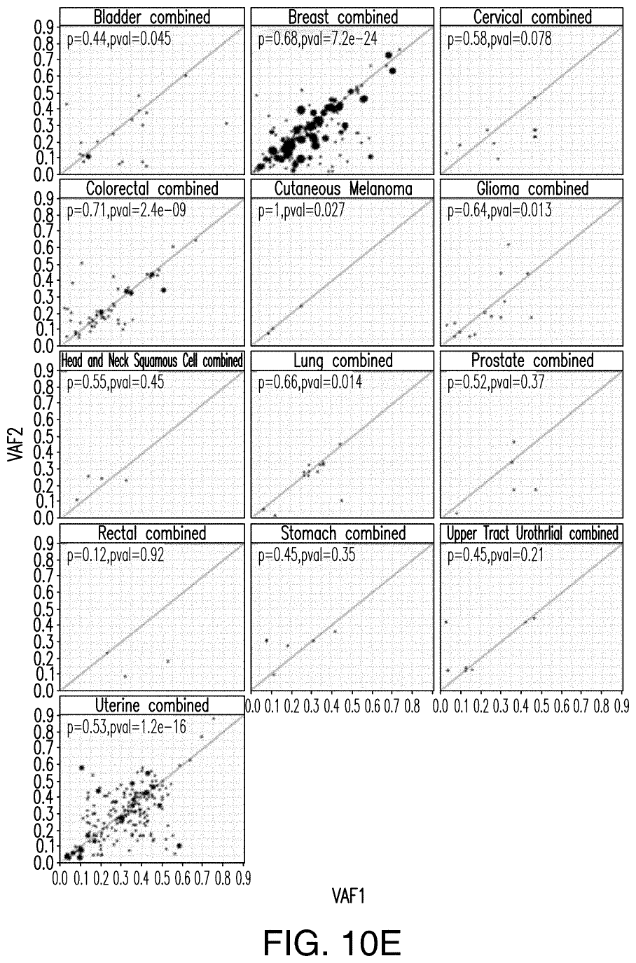

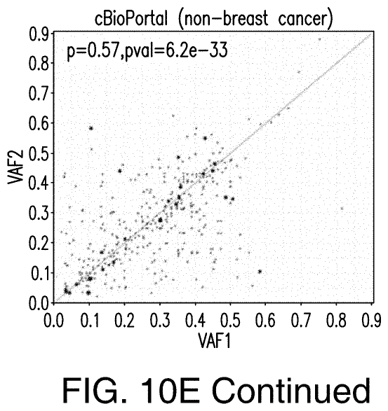

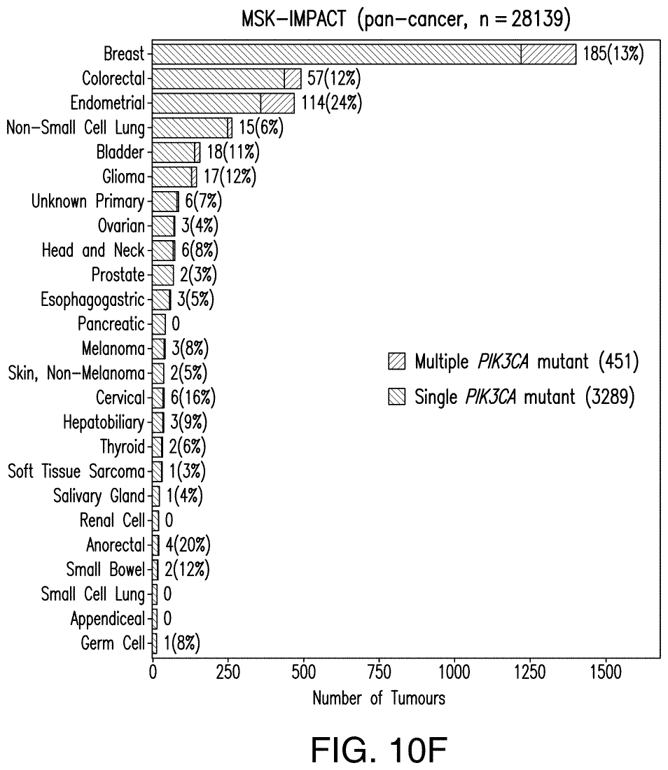

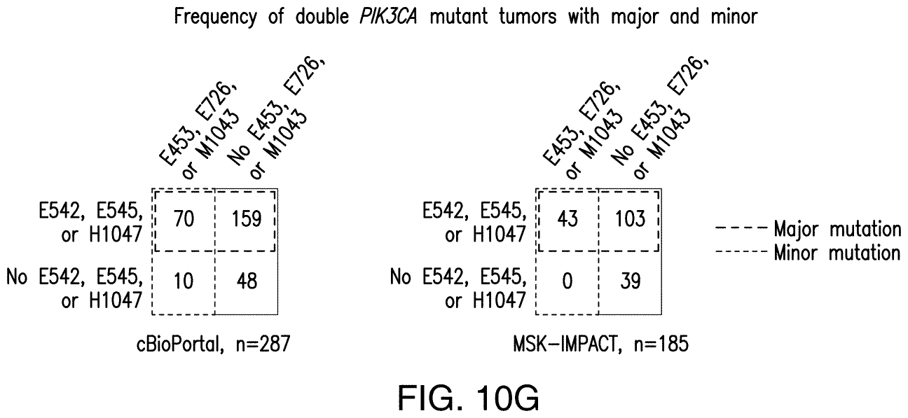

The present example analyzed a publicly available cohort of tumors across all cancer histologies (n=70754) from cBioPortal (Cerami, Cancer Discov, 2012, 2, 401-404; Gao, Sci Signal, 2013, 6, pl1). The present example identified 4530 PIK3CA-mutant tumors, 580 (12.8%) of which contain multiple PIK3CA mutations (FIG. 9A). The present example recapitulated these findings using a cohort of tumors across all cancer histologies (n=28139) sequenced by MSK-IMPACT (Cheng, J Mol Diagn, 2015, 17, 251-264), a targeted exome deep sequencing platform routinely used in the center. Among PIK3CA-mutant tumors (n=3745), 456 (12%) contained multiple PIK3CA mutations (FIG. 10A). In both cBioPortal and MSK-IMPACT datasets, breast cancer, colorectal cancer, and uterine cancer, had the greatest number of multiple PIK3CA-mutant tumors. The present example also analyzed individual breast cancer subsets of the cBioPortal dataset and found similar frequencies of multiple PIK3CA-mutant breast cancer in METABRIC (14%) and TCGA (12%) (FIG. 10B). The vast majority of multiple PIK3CA-mutant tumors in cBioPortal (88%) and MSK-IMPACT (89%) are comprised of exactly two mutations, which the present example termed “double hit” mutations (FIG. 10C).

The present example performed codon enrichment analysis to determine whether certain amino acid substitutions are found more frequently in multiple mutant tumors compared to single mutant tumors by Fisher's exact test. In the cBioPortal breast cancer dataset, E726, E453, M1043, E108, and K111 mutations were most frequently found in multiple mutant tumors compared to single mutant tumors (FIG. 9B). In the MSK-IMPACT breast cancer cohort, E726, E453, M1043, E88, P539, and E418 mutations were most frequently found in multiple mutant tumors compared to single mutant tumors (FIG. 10D). Thus, E726, E453, and M1043 are the most frequent recurrent mutations in double hit mutant breast tumors (FIG. 9C).

The present example found that 70/80 (88%) of multiple PIK3CA-mutant breast tumors from cBioPortal containing the E726, E453, and M1043 substitutions had a first site mutation involving the major hotspots E542, E545, or H1047 (data not shown). In both the cBioPortal and MSK-IMPACT non-breast cancer cohorts, E88 and E93 were however the most frequent mutations; and neither E726, E453, nor M1043 mutations were significantly enriched in this group (FIG. 9C and FIG. 10D). Thus, the most frequent dual PIK3CA mutant tumor combinations in breast cancer are comprised of a canonical “major mutant” hotspot (involving either E542, E545, or H1047) combined with a second “minor mutant” site (involving either E453, E726, or M1043) (FIG. 9C), and these recurrent dual mutations are specific to breast cancer compared to other cancer histologies.

Given that variant allele frequencies (VAFs) of the two PIK3CA mutations in dual PIK3CA mutant tumors follow a 1:1 distribution (FIG. 10E), the present example hypothesized that dual PIK3CA mutations in breast cancer are clonal. The present example performed an analysis using a large clinically-annotated breast cancer cohort (n=1918) the present example previously reported (Razavi, Cancer Cell, 2018, 34. 427-438). The present example analyzed clonality using FACETS (Shen, Nucleic Acids Res, 2016, 44, e131) of double hit mutants in breast cancer comprised of E545K/H1047R major hotspots and E453/E726/M1043 minor mutations (n=43) and found that the majority (65%) of dual PIK3CA mutant tumors are clonal for both mutations. Of the additional cases, 17% have a major clonal and minor subclonal mutation, 6% a major subclonal and minor clonal mutation, and 12% two subclonal mutations (FIG. 9D). Dual PIK3CA mutations are more frequently found in hormone receptor-positive (HR+)/HER2− breast cancer, compared to the group of other receptor subtypes (including HR−/HER2+, HR+/HER2+, and triple negative breast cancers) (15.4% vs 5.4%, p=0.004) (FIG. 9E). The difference in frequency of dual PIK3CA mutant tumors between therapy-naïve primary tumors and metastatic tumors did not reach statistical significance (11.6% vs 15.7%, p=0.130) (FIG. 9E).

Taken together, the present analysis using different cancer sequencing databases demonstrates a 10-15% frequency of multiple PIK3CA mutations across all PIK3CA-mutant cancers. In breast cancer, double hit PIK3CA mutations are mainly clonal, enriched at recurrent amino acid positions of a major and minor hotspot mutation, and associated with ER+HER2-primary and metastatic tumors.

Dual PIK3CA Mutations in Breast Cancer are Compound Mutations

Dual mutations can be compound (i.e. in cis on the same allele, coding for a single protein with two mutations) or biallelic (i.e. in trans, on separate alleles, coding for multiple proteins with different mutations). Given the clonality levels of the dual mutants and the 1:1 VAF distribution, the present example hypothesized that dual PIK3CA mutations are compound mutations.

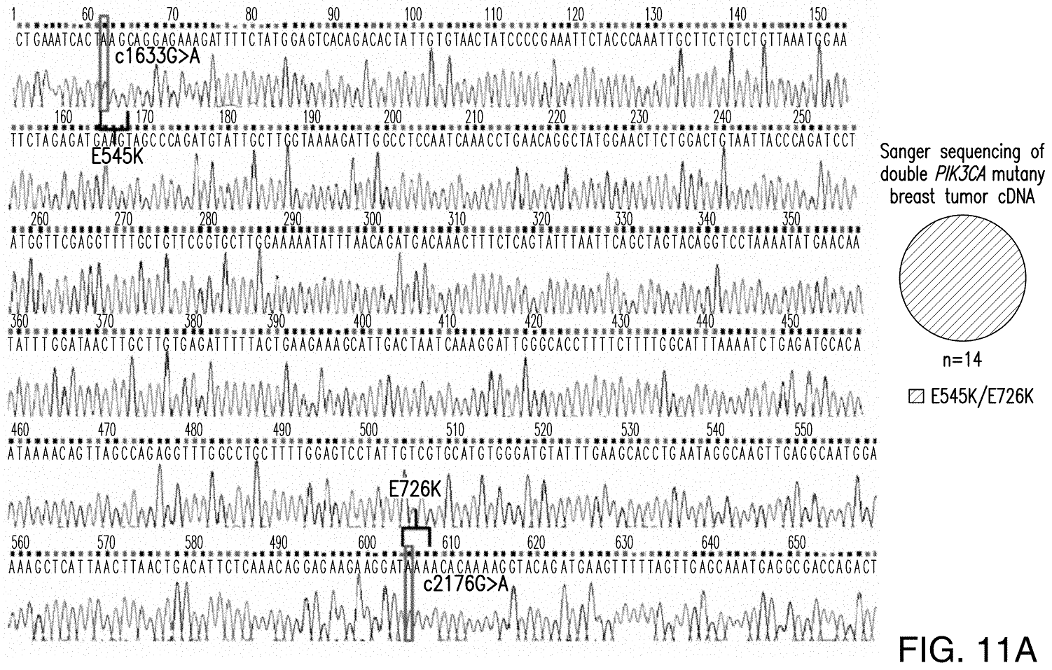

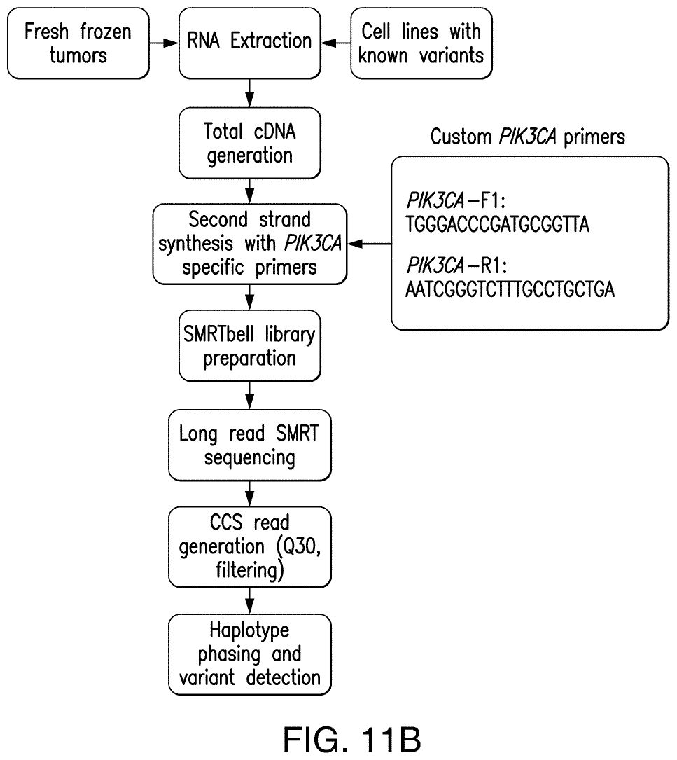

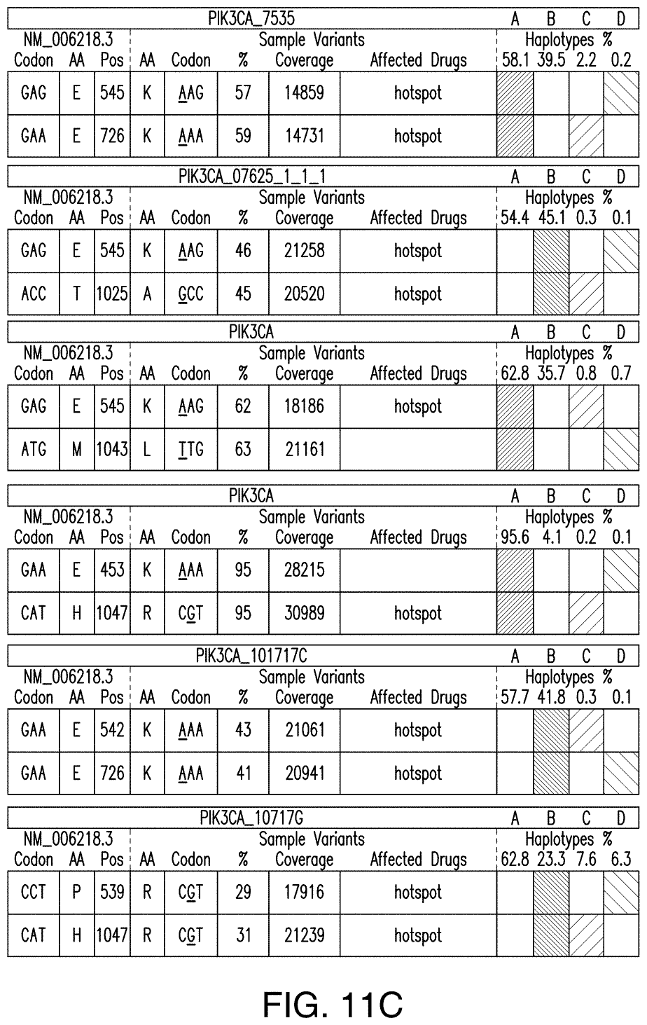

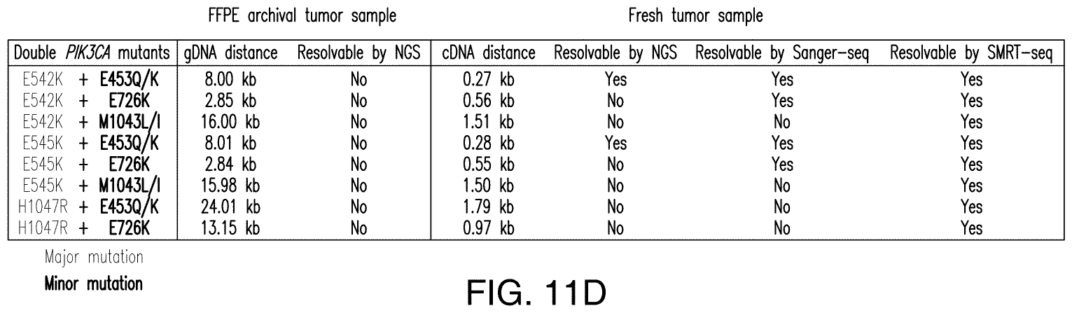

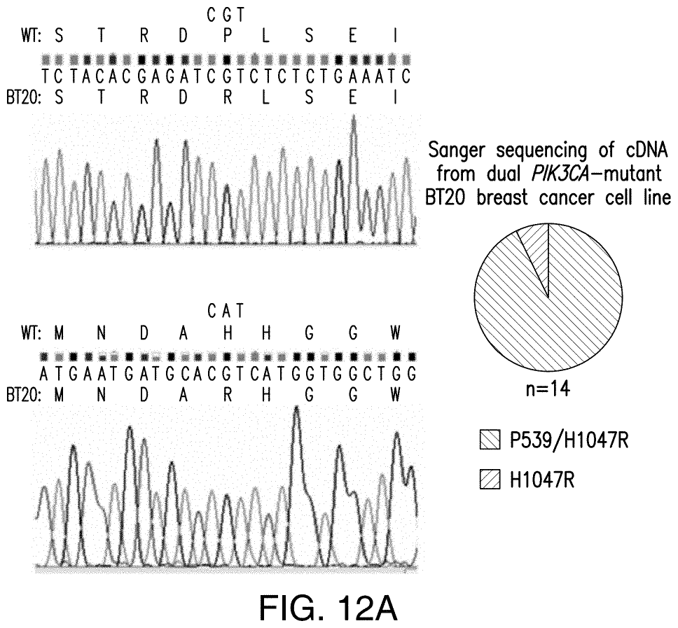

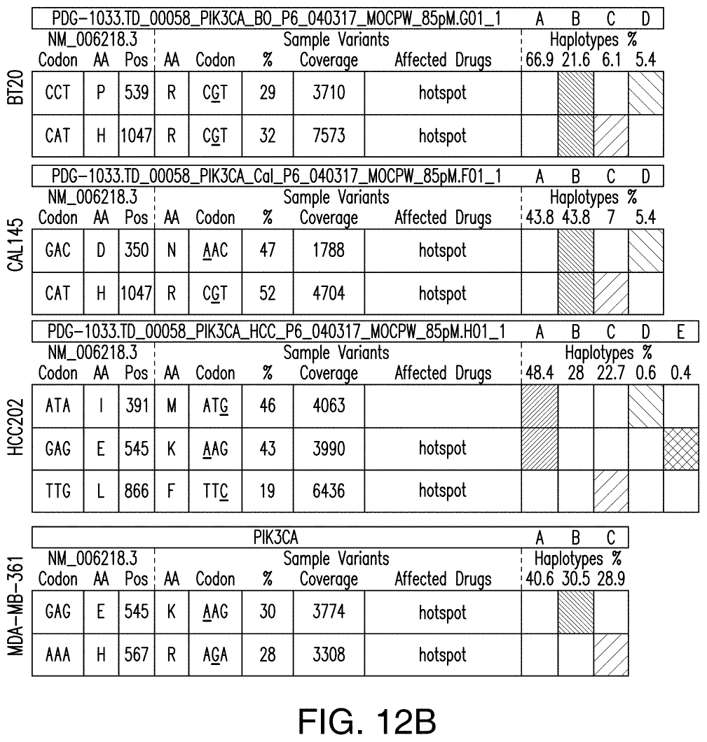

The most statistically significant dual mutant combinations in breast cancer (FIG. 9C) are located far apart in the gene. The majority of archival tumor specimens are preserved as formalin fixed, paraffin embedded (FFPE) samples, and this process shears genomic DNA and RNA to −200 nucleotide fragments, prohibiting phasing of the allelic configuration of recurrent breast cancer dual PIK3CA mutations. The present example overcame this dependence on FFPE samples by obtaining fresh frozen tumor samples. Notably, additional tumor tissue could be obtained only on patients with metastatic disease, diminishing the number of prospective patients by half since dual compound mutants are found equally in primary and metastatic tumors and since the majority of patients who underwent primary breast tumor resection were cured of their cancer. Samples were initially identified by MSK-IMPACT to contain dual mutants and then fresh frozen biopsies were obtained on the prospective biospecimen protocol. After obtaining the first dual mutant breast tumor E545K/E726K with high VAF, the present example performed bacterial colony Sanger sequencing and found that 14/14 (100%) of mutant inserts contained compound E545K and E726K mutations in cis (FIG. 11A). The present example identified four dual PIK3CA mutant breast cancer cell lines including BT20 (PIK3CA P539R and H1047R), both of whose hotspot mutations have been shown to be activating (Gymnopoulos, Proc Natl Acad Sci USA, 2007, 104, 5569-5574). The present example amplified full length PIK3CA from cDNA derived from BT20, subcloned the PCR products into the pGEM-T vector, and sequenced individual bacterial colonies by Sanger sequencing. 13/14 (92%) BT20-derived mutant inserts contained the P539R and H1047R mutations in cis (FIG. 12A).

While bacterial colony Sanger sequencing can be used to determine the allelic configuration of dual mutants, it is a heterologous system, exhibits low efficiency in tumors and biopsies with low cancer cell fraction, and is indirect for some dual mutants far apart in the gene as separate priming reactions would have to be performed. The present example adapted this workflow to include single molecule real-time sequencing (Eid, Science, 2009, 323, 133-138) (SMRT-seq) (FIG. 11B), which utilizes long range sequencing of circular DNA templates, enabling direct phasing of the allelic configuration of dual PIK3CA mutants far apart in the gene, from fresh breast tumor samples.

As controls, the present example analyzed by SMRT-seq four dual PIK3CA mutant breast cancer cell lines whose allelic configurations are not reported (FIG. 12B): BT20 (P539R/H1047R), CAL148 (D350N/H1047R), HCC202 (E545K/L866F), and MDA-MB-361 (E545K/K567R). BT20 contains compound mutations in 21.6% of amplicons by SMRT-seq, corroborating the previous Sanger sequencing data. CAL148 contains compound mutations in 43.8% of amplicons. HCC202 contains biallelic E545K and L866F mutations, but also contains compound E545K and I391M mutations in 48.4% of amplicons. MDA-MB-361 contains biallelic E545K and K567R mutations. Thus, the present example concluded that SMRT-seq is feasible to phase the allelic configuration of PIK3CA mutations, re-curate known cell line mutations, and discover additional genomic complexities.

The present example then obtained six additional fresh frozen breast tumors (previously confirmed to contain dual mutations by MSK-IMPACT) for SMRT-seq analysis. Importantly, this cohort contains samples from patients with E453, E726, and M1043-containing dual mutant combinations. All six patient tumors (100%) contain compound PIK3CA mutations by SMRT-seq (FIG. 11C).

The present example also used next generation sequencing (NGS) by MSK-IMPACT to interrogate the allelic configuration of PIK3CA mutations located close together in the gene. The present example phased ten PIK3CA compound mutant breast tumors in cis on the same allele (Table 1), and one PIK3CA biallelic mutant breast tumor in trans on separate alleles (Table 2). The present example also phased two PIK3CA compound mutant breast tumors in cis on the same allele from TCGA (Cancer Genome Atlas, Nature, 2012, 490, 61-70) using RNA sequencing data (Table 3). By NGS alone, only 6% of dual PIK3CA mutant breast tumors could be definitively phased.

TABLE 1
Dual PIK3CA mutant tumors phased as compound
mutants in cis, by MSK-IMPACT next generation
sequencing, from the MSK-IMPACT cohort.

| | alleles | |
|---|---|---|
| sample | (gDNA, in cis) | disease |
| P-0012667-T01-IM5 | E3B5K + D390N | Breast Cancer |
| P-0001902-T01-IM3 | E542K + E545D | Breast Cancer |
| P-0014479-T01-IM6 | E542Q + E545K | Breast Cancer |
| P-0029802-T01-IM6 | E542K + E545K | Breast Cancer |
| P-0022021-T01-IM6 | E545K + D549H | Breast Cancer |
| P-0024420-T01-IM6 | E545K + D549N | Breast Cancer |
| P-0026885-T01-IM6 | M1004I + H1047R | Breast Cancer |
| P-0000107-T01-IM3 | D1017E + I1019V + Y1021H | Breast Cancer |
| P-0021201-T01-IM6 | L1036S + M1040T | Breast Cancer |
| P-0021040-T01-IM6 | H1047R + M1055L | Breast Cancer |

TABLE 2
Dual PIK3CA mutant tumors phased as biallelic
mutants in trans, by MSK-IMPACT next generation
sequencing, from the MSK-IMPACT cohort.

| | alleles | |
|---|---|---|
| sample | (gDNA, in trans) | disease |
| P-0020645-T01-IM6 | M1043V + H1047R | Breast Cancer |

TABLE 3
Dual PIK3CA mutant tumors phased as compound mutants
in cis, by RNA-sequencing, from the TCGA cohort.

| | | | number | |
|---|---|---|---|---|
| | | | of reads | number |
| | | | calling | of reads |
| | alleles | | both | spanning |
| sample | (RNA, in cis) | disease | mutations | both loci |
| TCGA-AO- | M1004I + H1047R | Breast | 11 | 12 |
| A1KR-01A- | | Cancer | | |
| 12D-A142-09 | | | | |
| TCGA-A2- | H1047R + H1065L | Breast | 4 | 10 |
| A0EN-01A- | | Cancer | | |
| 13D-A099-09 | | | | |

These findings, obtained through multiple orthogonal sequencing techniques, support that double hit PIK3CA mutations are mainly found as compound mutations in breast cancer.

Compound PIK3CA Mutations Activate the PI3K Pathway More than Single Mutants

The present example next asked whether PIK3CA compound mutations result in a PI3K enzyme that activates the downstream pathway to a greater degree than single major or minor hotspot mutants. Given the frequency of combinations of major hotspot and minor hotspot mutations in breast cancer, and that E542K and E545K are predicted to have the same mechanism of activation (Zhao, Proc Natl Acad Sci USA, 2008, 105, 2652-2657), the present example focused on the compound mutants E453K/E545K, E453K/H1047R, E545K/E726K, E726K/H1047R, and E545K/M1043L and their constituent single mutants. The present example overexpressed each single and compound mutant using a low-copy number lentiviral expression system (pLX-302). The present example cloned PIK3CA without affinity tags, as N-terminal tags artificially increase kinase activity and C-terminal tags may interfere with membrane binding (Sun, Cell Cycle, 2011, 10, 3731-3739; Hon, Oncogene, 2012, 31, 3655-3666). The present example obtained stable clones in MCF10A breast epithelial cells and NIH-3T3 fibroblasts, both of which have been previously used to characterize PIK3CA mutations (Ikenoue, Cancer Res, 2005, 65, 4562-4567; Isakoff, Cancer Res, 2005, 65, 10992-11000). The present example also obtained stable clones from MCF7 ER+ breast cancer cells engineered to carry a PIK3CA wildtype (WT) background by somatic gene editing (Beaver, Clin Cancer Res, 2013, 19, 5413-5422).

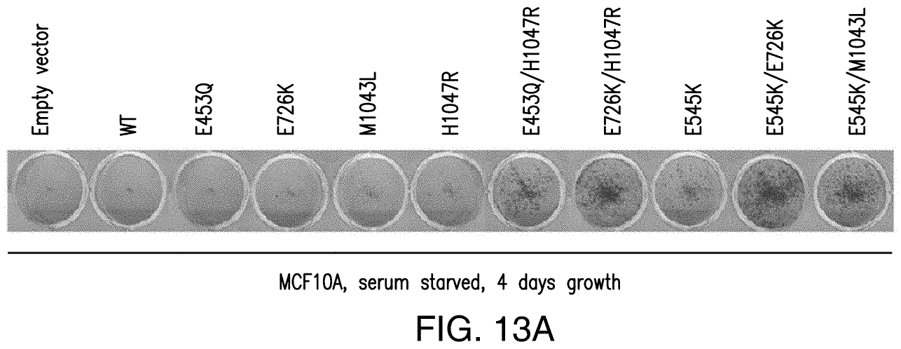

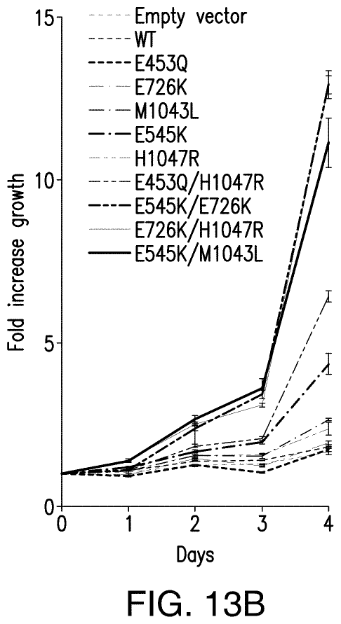

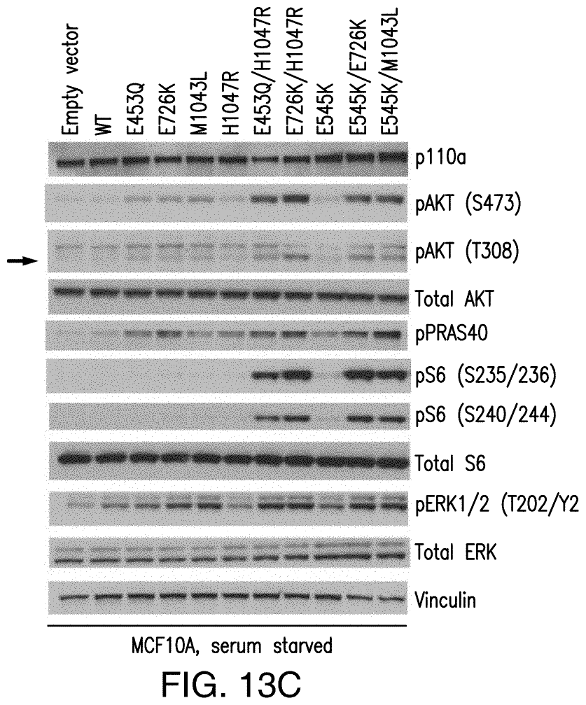

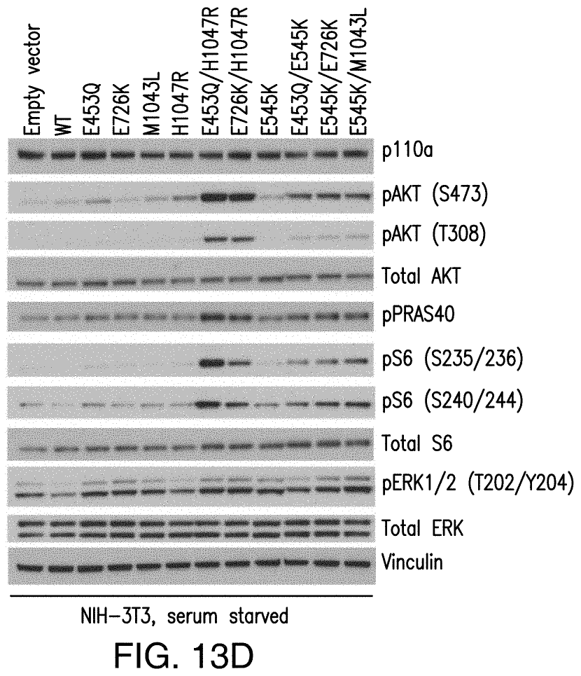

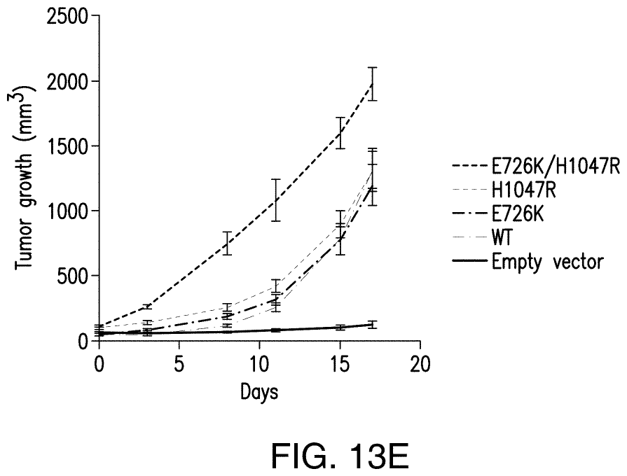

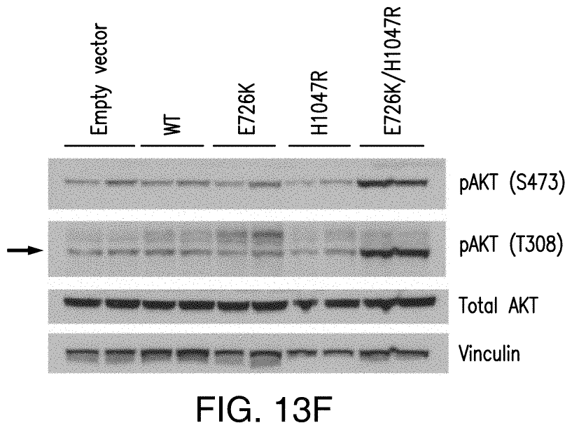

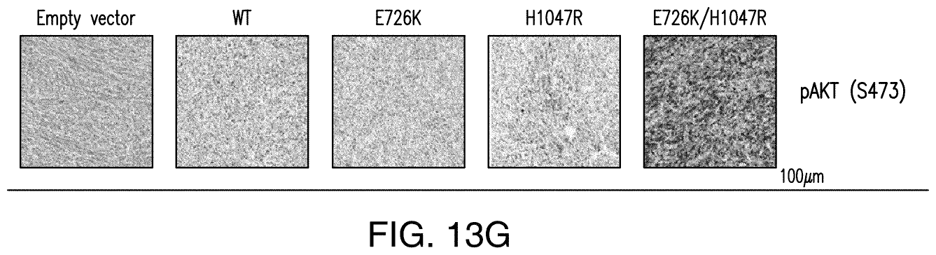

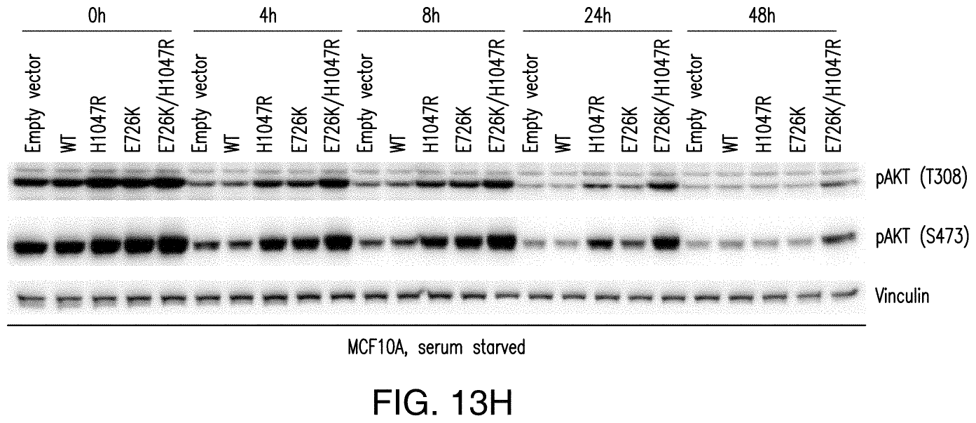

The present example measured basal growth proliferation over time of compound PIK3CA mutant MCF10A cells in medium containing serum but lacking EGF or insulin. All dual compound mutants (E453Q/H1047R, E545K/E726K, E726K/H1047R, E545K/M1043L) exhibited increased growth proliferation as compared to their constituent major (E545K or H1047R) or minor (E453Q, E726K, or M1043L) single mutants (FIGS. 13A-13B).

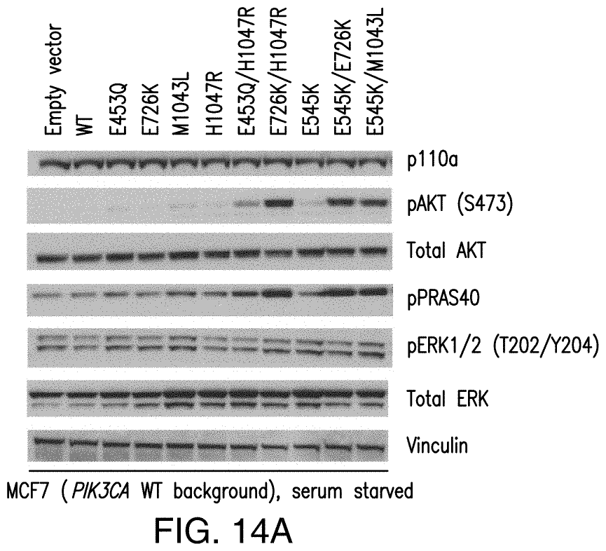

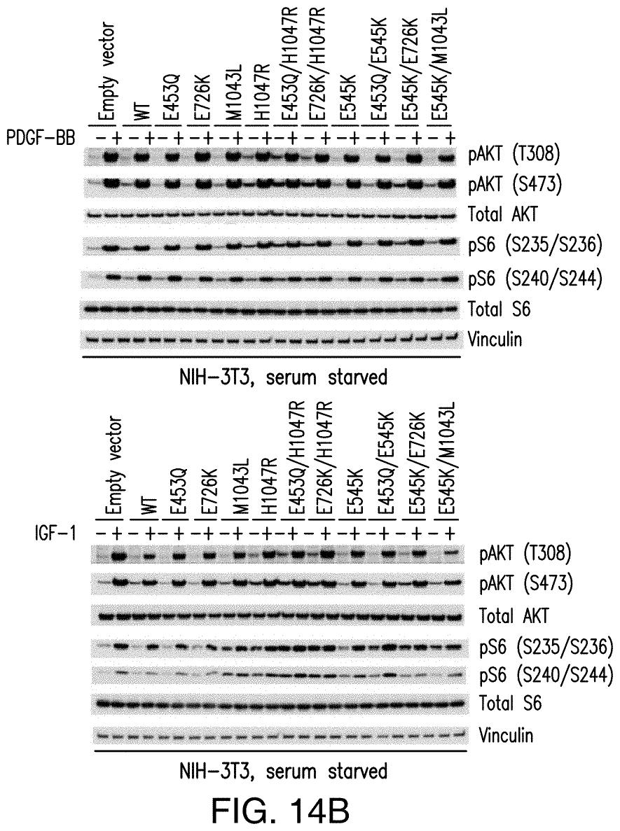

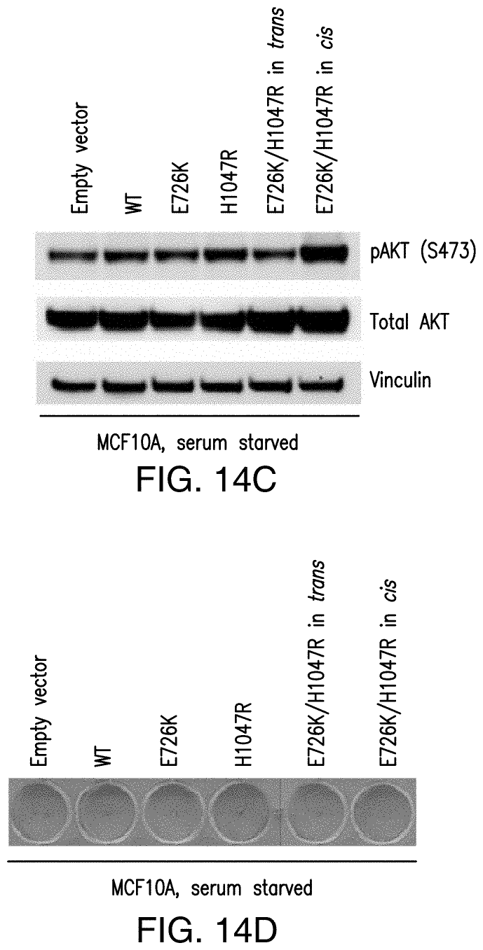

The present example measured PI3K pathway signaling in the MCF10A and NIH-3T3 nontransformed and MCF7 transformed cellular models. Compound PIK3CA mutations increased downstream PI3K pathway signaling more than single hotspot mutants, as evidenced by increased phosphorylation of pAKT (T308), pAKT (S473), pPRAS40, pS6 (S235/236), and pS6 (S240/244) in MCF10A and NIH-3T3 cells under serum starvation (FIGS. 13C-13D). Compound PIK3CA mutations also increased downstream PI3K pathway signaling more than single hotspot mutants, as evidenced by increased phosphorylation of pAKT (S473) and pPRAS40 in MCF7 cells under serum starvation (FIG. 14A). In MCF10A and MCF7 cells, E545K and E545K-containing compound mutants exhibit greater signaling than H1047R or H1047R-containing compound mutants, while in NIH-3T3 fibroblasts, H1047R and H1047R-containing dual mutants exhibit greater signaling than E545K or E545K-containing compound mutants, consistent with prior studies on PIK3CA signaling in fibroblasts (Zhao, Proc Natl Acad Sci USA, 2008, 105, 2652-2657). There were no consistent changes in pERK levels between single and compound mutants in any of the cell lines tested.

The present example next investigated PI3K activation in vivo positing that dual compound PIK3CA mutant cells enhance tumor growth in vivo compared to single mutants. The present example chose the E726K/H1047R compound mutant since it exhibited the highest amount of PI3K signaling in vitro. E726K/H1047R compound mutant NIH-3T3 xenografts demonstrate increased tumor growth compared to H1047R, E726K, WT, and empty vector (FIG. 13E). There was no difference in tumor growth between the single mutants and WT. E726K/H1047R compound mutant NIH-3T3 tumors exhibited higher activation of the PI3K pathway compared to single mutants through increased phosphorylation of AKT (S473 and T308) on Western blotting (FIG. 13F) and increased staining for pAKT (S473) by immunohistochemistry (FIG. 13G).

Together, these data show that compound PIK3CA mutants activate PI3K pathway signaling and promote tumor growth to a greater degree than single mutants.

Compound PIK3CA Mutations Promote a More Open Conformation of PI3K than Single Mutants

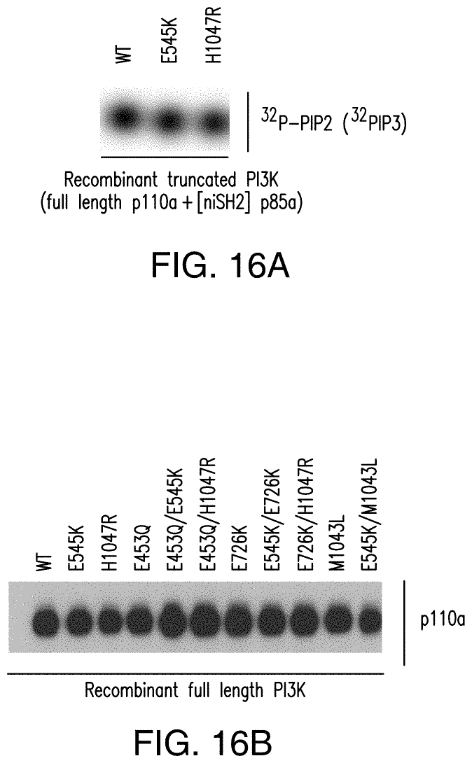

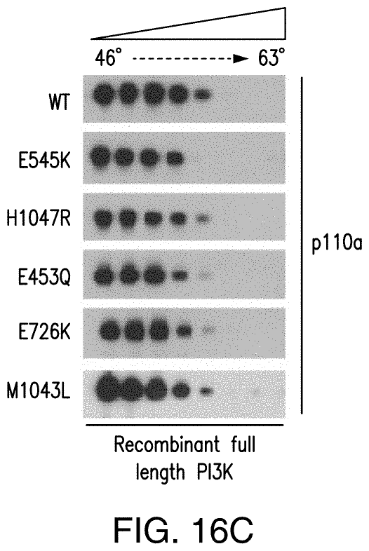

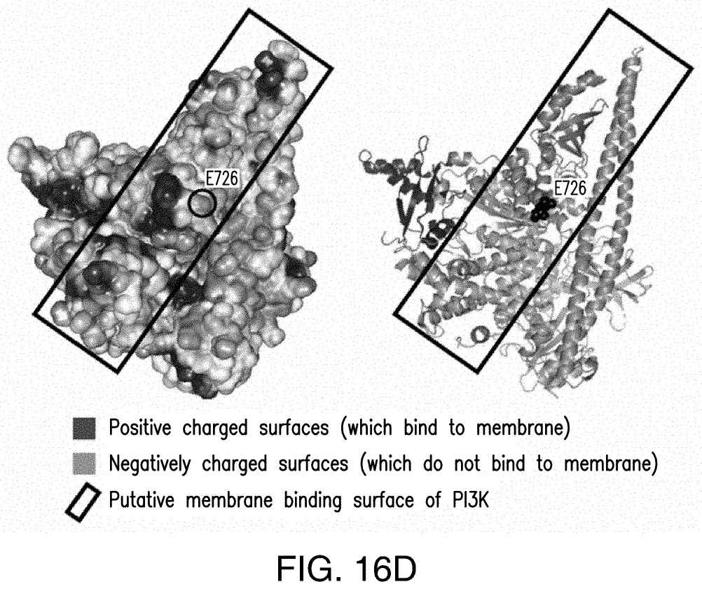

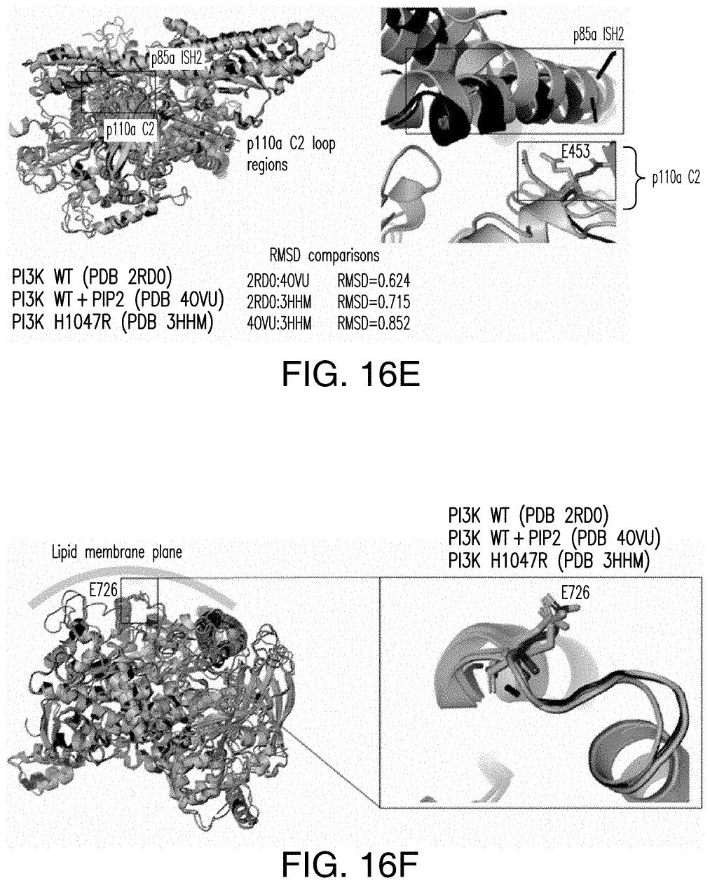

The present example then investigated the consequences of compound PIK3CA mutations on PI3K enzyme biochemistry. The present example initially purified truncated PI3K protein complexes comprising full length p110α and the niSH2 domain of p85α, corresponding to the crystallized truncated PI3K complex (Huang, Science, 2007, 318, 1744-1748), using baculoviral expression in insect cells in the presence of the PI3Kα inhibitor BYL719. Upon these conditions, the kinase activity was high and not altered in WT versus single and double mutant, likely due to the absence of the cSH2 domain of p85α which stabilizes and inhibits p110α (FIG. 16A).

The present example then expressed and purified recombinant full length human PI3Kα complexes (comprised of untagged p110α and hexahistidine-tagged p85α (“hexahistidine” is disclosed as SEQ ID NO: 19)) from EXPI293 human embryonic kidney cells in the absence of PI3K inhibitors (FIG. 16B) to investigate the effects of compound p110α mutations on protein complex stability, lipid binding, and lipid kinase activity. The prevailing model of PI3K activation by PIK3CA single oncogenic mutations (Hon, Oncogene, 2012, 31, 3655-3666; Huang, Science, 2007, 318, 1744-1748; Burke, Proc Natl Acad Sci USA, 2012, 109, 15259-15264; Mandelker, Proc Natl Acad Sci USA, 2009, 106, 16996-17001) classifies single mutants as mutants that destabilize and are disinhibited by p85α, which the present example term “disrupters,” and mutants that increase p110α membrane binding, which the present example term binders. E545K and E453Q are predicted to be disrupters, where E545K mimics phosphopeptide binding to the nSH2 domain of p85α, and E453Q impairs p110α C2 domain binding to the p85α iSH2 domain. H1047R and M1043L are predicted to be binders and are in the C-terminal membrane-binding tail. E726K has been reported to be activating (Zhang, Cancer Cell, 2017, 31, 820-832 e823) but its mechanism of action is still undetermined. The present example analyzed the membrane binding surface of PI3K based on its crystal structure (Miller, Oncotarget, 2014, 5, 5198-5208) (FIG. 16D) and hypothesized that E726K is also a binder as the mutant lysine would increase positive charge and promote membrane binding to negatively charged phospholipids. The present example speculated that compound PIK3CA mutations increase PI3Kα protein complex destabilization, lipid binding, and lipid kinase activity to a greater degree than single minor or major mutants.

PI3Kα complex destabilization and disinhibition has been measured using hydrogen-deuterium exchange mass spectrometry, where increased deuterium exchange corresponds with increased destabilization and a more open conformation of the enzyme complex (Burke, Proc Natl Acad Sci USA, 2012, 109, 15259-15264) and also through molecular dynamic simulations (Echeverria, FEBS J, 2015, 282, 3528-3542). The present example modeled destabilization using thermal shift assays, where increasing temperature promotes exposure of the hydrophobic core of a protein resulting in its aggregation. Proteins that are more intrinsically unstable will aggregate at a lower temperature, and this can be measured by Western blotting. In the case of the heterodimeric PI3K complex, monomeric p110α that forms as a result of mutant p110α destabilization from p85α is intrinsically unstable, leading to its aggregation (Yu, Mol Cell Biol, 1998, 18, 1379-1387). The present example took advantage of this phenomenon by adapting thermal shifts to measure basal PI3K compound mutant complex destabilization, where complexes are heated on a temperature gradient, and supernatants are separated by centrifugation, probed with anti-p110α antibody across the temperature range, and measured for the temperature at which soluble p110α is decreased in the supernatant.

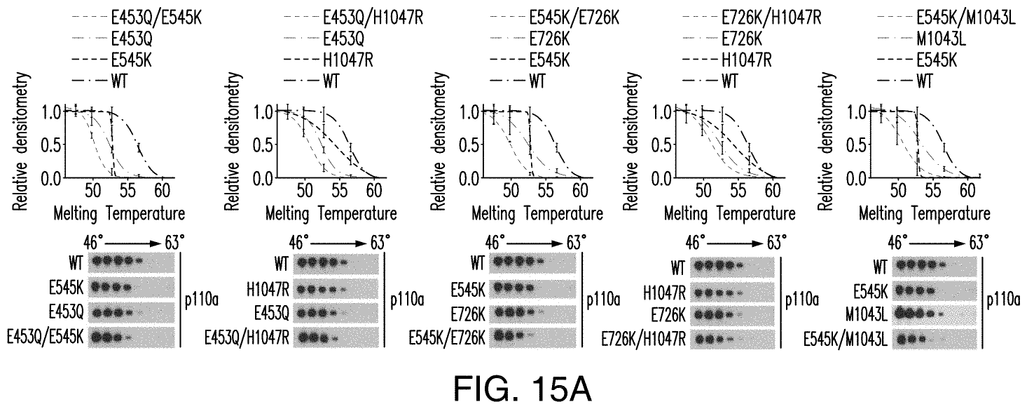

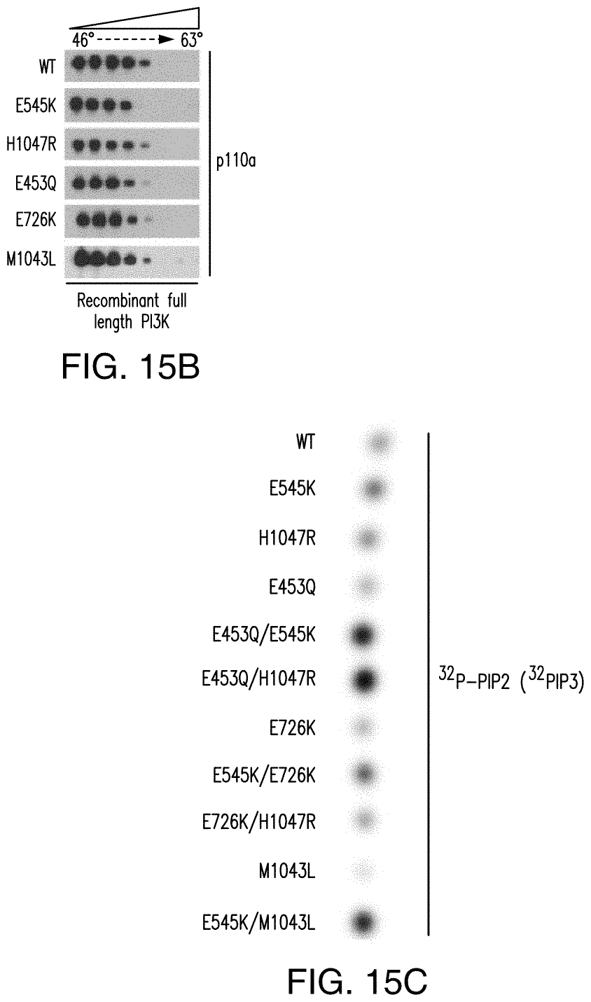

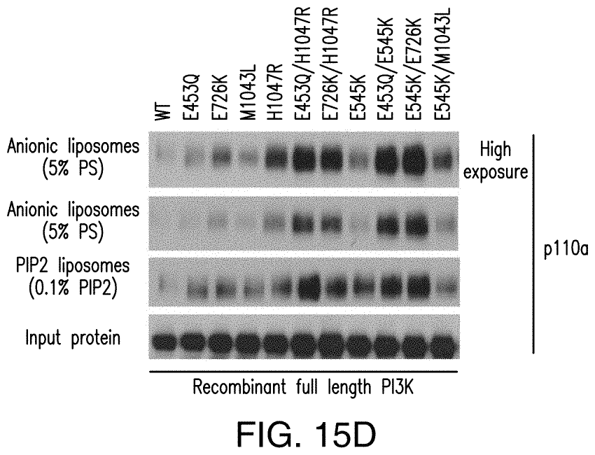

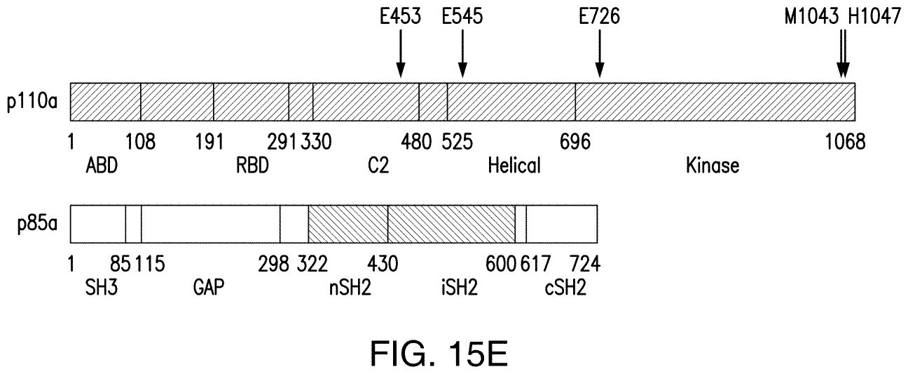

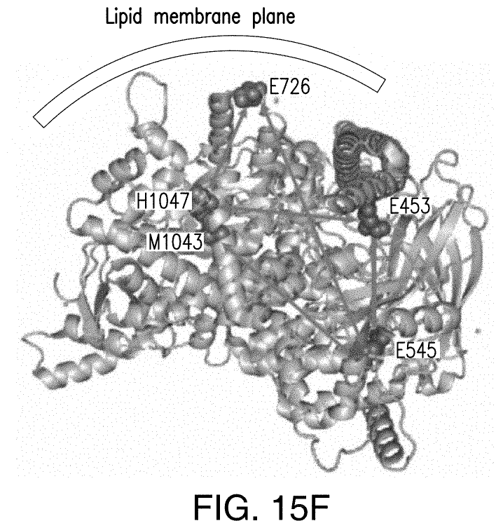

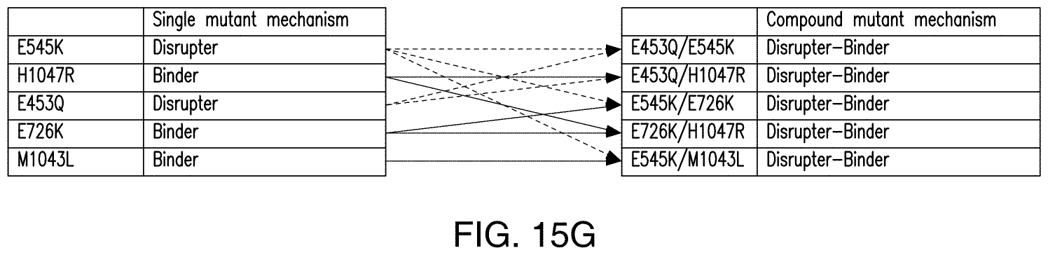

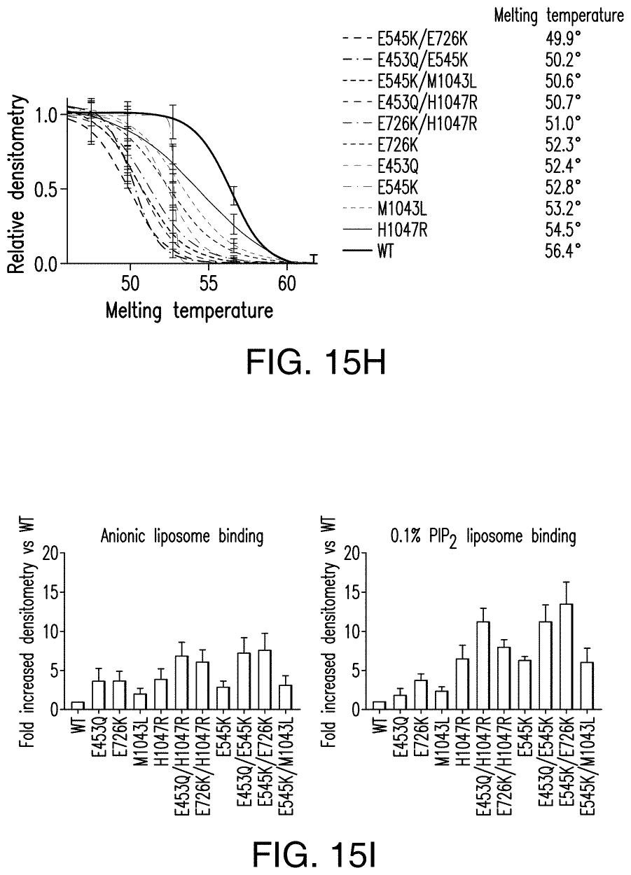

The compound mutants E453Q/E545K, E453Q/H1047R, E545K/E726K, E726K/H1047R, and E545K/M1043L demonstrate increased thermal instability compared to each of their constituent minor and major mutants (FIG. 15A). Among single mutants, E545K is the most thermally unstable while H1047R and M1043L, whose most salient biochemical functions are lipid binding, still exhibit some thermal instability compared to WT PI3K (FIG. 15B). The other minor mutants exhibit an intermediate thermal instability phenotype compared to E545K and H1047R (FIG. 16C). Thus, the present example concludes that the single major and minor mutants all are disrupters.

Compound PIK3CA Mutations Increase Lipid Binding and Kinase Activity

The present example next used the recombinant proteins to measure basal kinase activity. The present example assessed the levels of PIP3, the product of the PI3K lipid kinase reaction, by measuring the production of radiolabeled ³²P-labeled PIP3 by thin-liquid chromatography (TLC) based lipid kinase assays. E453Q/E545K, E453Q/H1047R, and E545K/M1043L demonstrated increased basal kinase activity compared to each of their constituent minor and major mutants (FIG. 15C).

To assess whether compound mutants increase lipid binding as a consequence of increased destabilization and exposure of membrane-binding protein surfaces, the present example used the recombinant proteins to perform liposome binding assays using neutral liposomes and also liposomes containing 0.1% PIP2. The present example measured the amount recombinant protein complexes that bound to liposomes by Western blotting for p110α. All compound mutants tested (E453Q/E545K, E453Q/E1047R, E545K/E726K, E726K/H1047R) exhibit increased binding to neutral liposomes compared to single major or minor mutants (FIG. 4E). E453Q/E545K, E453Q/E1047R, and E545K/E726K compound mutants demonstrated enhanced binding to PIP2 liposomes. All single mutants increased liposome binding compared to WT. Thus, the single major and minor mutants all are binders. Overall, PI3Kα complexes exhibited increased binding to PIP2-containing liposomes compared to control liposomes, with single mutants displaying a PIP2-dependent increase (FIG. 15E).

Together, this biochemical data demonstrates that double hit compound PIK3CA mutations in breast cancer function through a combination protein disrupter-membrane binder mechanism (FIG. 15F).

Compound PIK3CA Mutations are Preferentially Inhibited by PI3K Inhibitors

Given the mechanistic data that compound PIK3CA mutations hyperactivate the PI3K protein and signaling pathways, the present example investigated the effects of the PI3K inhibitor BYL719 on compound PIK3CA mutations. Given that compound mutants exhibit increased dependence on the PI3K pathway, the present example predicted that they would be more inhibited by PI3K inhibitors.

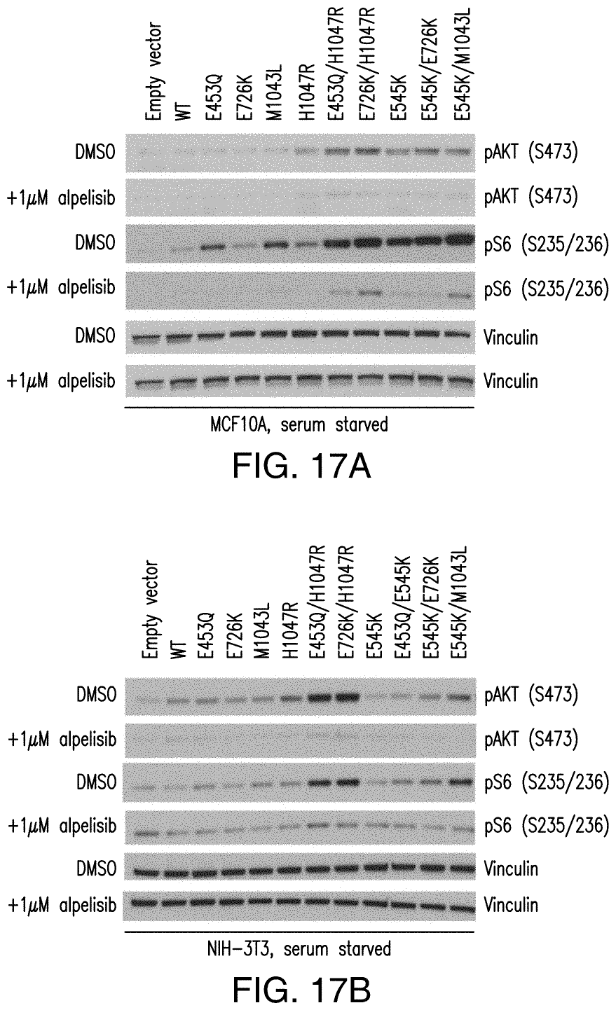

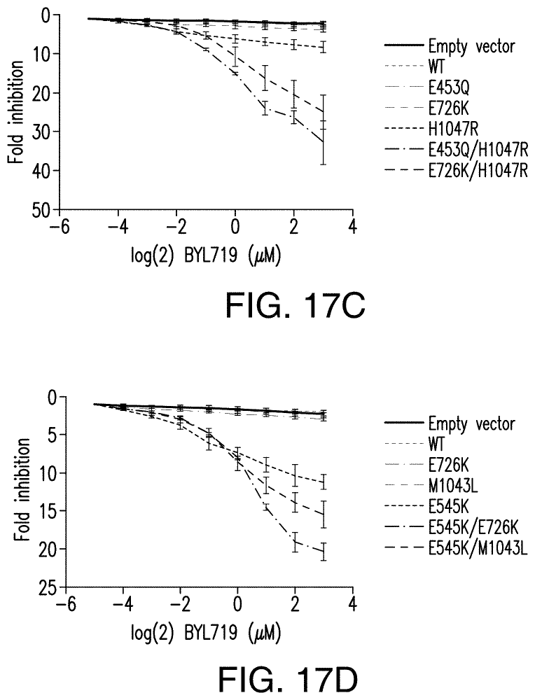

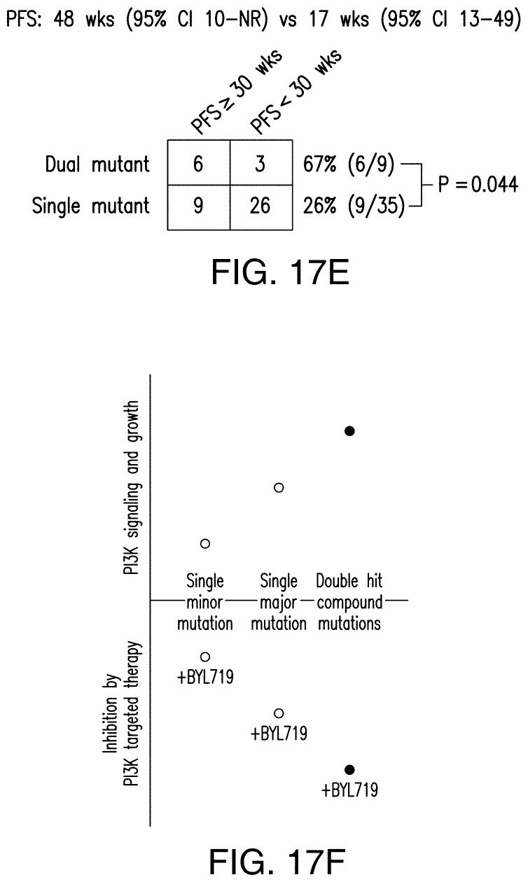

The present example measured inhibition of PI3K pathway signaling by BYL719 by exposing cells to inhibitor for 24 hours under serum starvation. While in the absence of pharmacological pressure compound mutant signaling is increased compared to single mutants, on PI3K inhibition, compound mutant signaling decreases to similar levels as single mutant cells in MCF10A (FIG. 17A) and NIH-3T3 models (FIG. 17B). The present example used the MCF10A cell culture models to test the levels of cell growth inhibition by BYL719. E545K-(FIG. 17C) and H1047R- (FIG. 17D) containing compound mutants demonstrate increased fold of inhibition to BYL719.

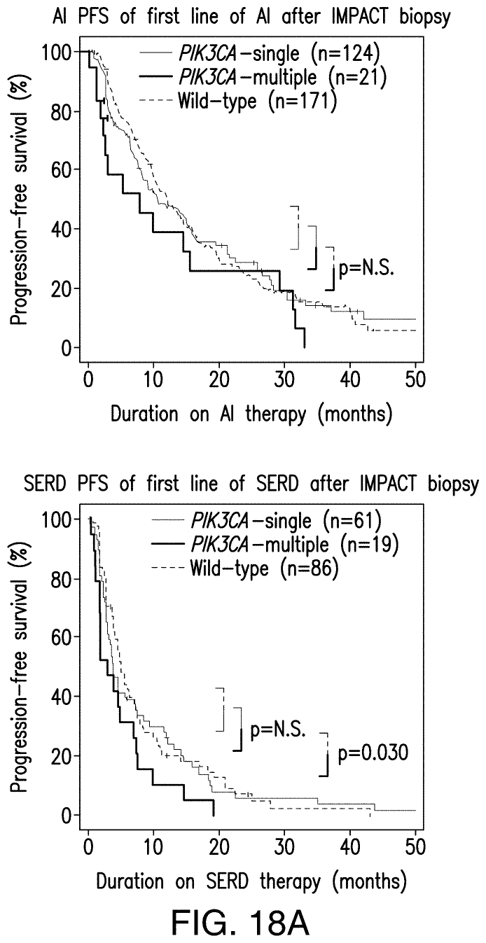

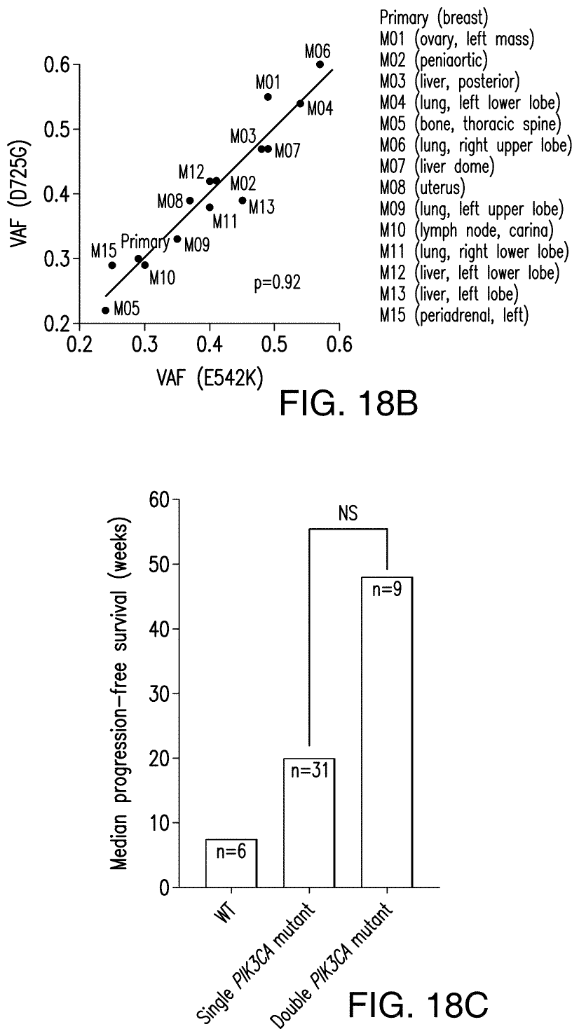

Given the exquisite dependence of dual PIK3CA mutants on the PI3K pathway, the present example hypothesized that dual PIK3CA mutations are predictive of improved clinical duration of response to PI3K inhibitor therapy compared to single hotspot PIK3CA mutations in ER+ breast cancer patients. The present example performed a re-analysis of patients enrolled on a phase 1 clinical trial investigating the efficacy of BYL719 in combination with an aromatase inhibitor in heavily pretreated patients with ER+ metastatic breast cancer (NCT 01870505). The present example sequenced both tumors and circulating tumor DNA using NGS from 9 patients on the trial with dual PIK3CA mutations. Dual mutant patients responded longer to PI3K inhibition than single mutant patients (48 weeks vs 17 weeks, 95% CI 10 weeks-not reached vs 13-49 weeks), but this was not statistically significant, likely due to small numbers (FIG. 17E). The present example made a cut point based on the PFS of patients on the SANDPIPER trial (7.4 months total=30 weeks) (Baselga, Journal of Clinical Oncology, 2018, 36, no. 18_suppl.). 67% of patients with dual mutant tumors had an increased clinical benefit rate >30 weeks compared to 23% of patients with single mutant tumors, which was statistically significant (p=0.044) (FIG. 17E). Given that the majority of ER+ breast cancer patients receive endocrine therapies, the present example retrospectively interrogated whether dual PIK3CA mutations are predictive of improved response to antiestrogen therapies. Patients with dual PIK3CA-mutant tumors do not have improved progression-free survival (PFS) when treated with aromatase inhibition or fulvestrant as compared to patients with single mutant or WT tumors (FIG. 18A), and dual mutant patients have worse PFS to fulvestrant compared to patients with WT tumors.

### DISCUSSION

In this work, the present example has discovered and characterized double hit compound mutations in PIK3CA, the most frequently mutated oncogene in cancer. These findings establish that compound mutations activate the PI3K pathway to a greater degree than single major hotspot mutants (FIG. 17F). These findings indicate that compound mutations acquire both the combined protein destabilizing and membrane binding properties of single mutants. This stepwise pattern of activation is also reflected in drug sensitivity, where compound mutations are more inhibited by the PI3K inhibitor BYL719 than major mutations (FIG. 17F).

PIK3CA major hotspot mutations activate PI3Kα; however, very little is known of the biological or clinical relevance of minor PIK3CA mutations, which represent ˜60% of PIK3CA mutations (Zhang, Cancer Cell, 2017, 31, 820-832 e823). The present analysis of the entire corpus of publicly available tumor sequencing data has demonstrated a frequency of double hit mutations across PIK3CA mutant tumors of 10-15%. This frequency stands in stark contrast to prior estimates of dual PIK3CA mutations in PIK3CA-mutant tumors (<1%), likely due to incomplete sequencing across PIK3CA exons in those studies (Saal, Cancer Res, 2005, 65, 2554-2559; Yuan, Oncogene, 2008, 27, 5497-5510). Double hit PIK3CA mutations recur across the gene at varied minor mutant sites in breast versus non-breast tumors suggesting tissue dependent phenotypes for different double hit mutant genotypes. The present sequencing analyses revealed that double hit PIK3CA mutant breast tumors, including representative tumors containing E453, E726, and M1043 minor mutations, are compound mutations.

Functionally, the present example has shown that certain minor PIK3CA mutations have little capacity in activating the PI3K pathway, but they can synergize with major hotspot mutations in signaling and tumor growth. This is fundamentally different from the synergistic oncogenic phenotype observed by artificially engineering as compound mutants the major hotspots E545K and H1047R, each of which already have enhanced activating capacity (Zhao, Proc Natl Acad Sci USA, 2008, 105, 2652-2657). Oncogenic PIK3CA mutations have been classified as “disrupters”, mutations that destabilize p85 binding and inhibition, and “binders”, mutations that increase membrane binding. These data show that double hit compound mutants act through a combination disrupter-binder mechanism (FIG. 15F). This is underscored by the fact that compound mutant combinations that are composed of constituent single mutants predicted to be pure disrupters (e.g. E453Q/E545K) or pure binders (e.g. E726K/H1047R) also demonstrate increased lipid binding or increased thermal instability, respectively (FIG. 15G). While all double hit compound mutants increased cellular signaling under serum starvation, not all recombinant compound mutants increased basal kinase activity. The increased open conformation of double hit compound mutants also raises the possibility of neomorphic functions such as additional protein binding partners.

The data demonstrate that tumors bearing compound PIK3CA lipid kinase mutations are more dependent on the resulting hyperactive PI3K pathway and are, consequently, exquisitely sensitive to PI3K inhibition. This is in contrast to compound protein kinase mutations that arise in the setting of acquired resistance to targeted therapies and frequently cause steric hindrance to drug (Khorashad, Blood, 2013, 121, 489-498; Shah, J Clin Invest, 2007, 117, 2562-2569; Kobayashi, J Thorac Oncol, 2013, 8, 45-51; Shaw, N Engl J Med, 2016, 374, 54-61). Many prior clinical trials investigating PI3K inhibitors have relied on partial sequencing platforms to determine if tumors had PIK3CA mutations, which may have obscured any differential responses in patients with double hit PIK3CA-mutant tumors. These findings merit retrospective correlative re-analysis of these trials. The present example speculates that double hit compound mutant PIK3CA can function as a clinical biomarker of increased sensitivity to PI3K-directed targeted therapies and may improve the therapeutic window of PI3K inhibitors in ER⁺ breast cancer and other PIK3CA-mutant tumor histology.

Methods

Mutational Data

All cases reported with PIK3CA mutation were downloaded from www.cbioportal.org. Ten breast cancer studies were analyzed within the Breast Cancer cohort. Those cases not found in METABRIC and TCGA were combined as Breast Cancers (others). Cell line and xenograft studies were removed in Breast and Pan Cancer cohorts.

The MSK IMPACT dataset consisted of 28139 tumor samples from patients who were prospectively sequenced as part of their active care at Memorial Sloan Kettering Cancer Center (MSKCC) between January 2014 and September 2018, as part of an Institutional Review Board-approved research protocol (NCT01775072). All patients provided written informed consent, in compliance with ethical regulations. The details of patient consent, sample acquisition, sequencing and mutational analysis have been previously published. Briefly, matched tumor and blood specimens for each patient were sequenced using Memorial Sloan Kettering-integrated mutation profiling of actionable cancer targets (MSK-IMPACT)—a custom hybridization capture-based next-generation sequencing assay approved for clinical use in New York state. All samples were sequenced with 1 of 3 incrementally larger versions of the IMPACT assay, including 341, 410, and 468 cancer-associated genes, respectively. The details of sample acquisition, sequencing and mutational analysis have been previously published (Zehir, Nat Med, 2017, 23, 703-713). All PIK3CA mutations were identified and tumors were identified as containing single, dual, or multiple PIK3CA mutations.

Codon Enrichment Analysis

PIK3CA single and dual mutant tumors were combined in the indicated cohorts. Tumors were analyzed for the frequency of a particular amino acid site mutation across the whole p110α protein in dual mutant tumors versus single mutant tumors, compared to chance, as assessed by Fisher's exact test. Statistics were calculated together for all studies.

Phasing Mutations and Clonality Analysis

To determine the allelic configuration of multiple somatic mutations in the same gene and tumor (i.e. to “phase” them), the present example implemented a computational framework for read-backed phasing. To this end, the present example exploited the fact that if two mutations were near enough in genomic position to be spanned by the same sequencing reads, then the identification of individual sequencing reads calling both variants at once unambiguously indicated that the different variants arose on the same DNA fragment, and therefore were in cis in the tumor genome. Conversely, if a large proportion of the reads spanning both mutations' loci called either mutation, but none call them both, and the two mutations were clonal enough to have arisen in the same cells, this implied that the two mutations arose in trans. Briefly, when two or more mutations in the same gene were found in a sample in tumor sequencing dataset, the tumor's raw sequencing data in BAM format was algorithmically queried using Samtools (version 1.3.1) (Li, H. et al. Bioinformatics 25, 2078-2079, (2009) for the reads mapping to the loci of each mutation in that gene. The unique barcodes for the individual read-pairs calling each mutant allele were then obtained using the sam2tsv function from jvarkit (Lindenbaum, FigShare, 2015, doi:10.6084/m9.figshare.1425030). By inspecting the barcodes calling the different mutant alleles in a gene, the present example called two mutations in cis if both mutations were called by the same read-pair (in at least two distinct read-pairs, to mitigate false positives due to sequencing error). Conversely, the present example called two mutations in trans if their loci were spanned by at least 10 reads, but less than two called them both at once, and their cancer cell fractions (as estimated by the FACETS algorithm (version 0.3.9) (Shen, Nucleic Acids Res, 2016, 44, e131) summed to at least 100%, indicating that they likely arose in the same cancer cells. FACETS was also used for clonality analyses on dual mutant tumors.

Fresh Frozen Tumor Acquisition

Patients were initially identified as having dual PIK3CA mutant tumors by MSK-IMPACT on FFPE samples, then were consented for collection of fresh tumor biopsies.

RNA Extraction and cDNA Generation

RNA was extracted from cell pellets (1×10⁷ cells) using the RNeasy Mini Kit (Qiagen), as specified by the manufacturer. Briefly, cells were homogenized in 350 μL lysis buffer (buffer RLT) by needle shearing, passing the resuspended pellet through a 20-gauge needle attached to a 5 mL syringe 10 times until a homogenous lysate was achieved. RNA extract from the lysate was then mixed with 70% ethanol and applied to the RNeasy spin column. Following the designated binding and wash steps, total RNA was eluted from the column twice using 30 μL RNAase free water for each elution, resulting in 60 μL extracted RNA per sample. Upon extraction, total RNA was aliquoted and stored at −80° C. for later use.

Total cDNA for SMRT-seq was generated using the SuperScript IV First Strand Synthesis System for RT-PCR (part no. 18091050; Thermo Fisher Scientific) using, 5 μL total RNA input, the provided oligo (dT) to prime first-strand synthesis and according to the manufacturer's protocol. Aliquots of cDNA were stored at −20° C. until needed for custom-primer, targeted PIK3CA amplification to achieve full-length molecules to phase variants of interest for diagnostic purposes. Total cDNA for Sanger sequencing was generated using the iScript cDNA Synthesis Kit (Bio-Rad).

Sanger Sequencing

BT20, CAL148, HCC202, and MDA-MB-361 cells were purchased from ATCC. Fresh frozen tumors were acquired from cancer patients, and samples were homogenized in RIPA buffer supplemented with protease and phosphatase inhibitors (Roche). Full length PIK3CA cDNA was amplified using Taq polymerase to generate 3′ A-tailed fragments and purified using a Qiaquick Gel Extraction kit (Qiagen). Full length PIK3CA cDNA was ligated into pGEM-T (Promega), transformed into E. coli, and plated on LB plates containing ampicillin, IPTG, and X-Gal for blue and white colony selection. White colonies were selected, miniprep plasmid DNA was isolated (Qiagen), and were submitted for Sanger sequencing.

PIK3CA Amplification for SMRT-Seq

Targeted PIK3CA amplification was performed using polymerase chain reaction (PCR) with High Performance Liquid Chromatography (HPLC)-purified primers: PIK3CA-F1: TGGGACCCGATGCGGTTA [SEQ ID No: 1]; and PIK3CA-R1: AATCGGTCTTTGCCTGCTGA [Seq ID No: 2]. The primers were synthesized at Integrated DNA Technologies, purified, and diluted to 10 μM in 0.1×TE buffer before use. Each reaction totaled 50 μL and consisted of 5 μL total cDNA, 5 μl 10×LA PCR Buffer II (Mg²⁺ plus), 8 μL of 2.5 mM dNTP mix, 2 μL each of PIK3CA-F and PIK3CA-R, 27.5 μL of nuclease free water, and 0.5 μL of LA-Taq polymerase (part no. RRO2C, TaKaRa Bio). Reactions were heated to 98° C. for 3 minutes and then subjected to 32 cycles of PCR using the following parameters: 25-sec denaturation at 98° C., followed by 15-sec annealing at 55° C., followed by 8-min extension at 68° C. After the 32nd cycle, the reactions were incubated for 15 min at 68° C. and then held at 4° C. PIK3CA amplicons were purified from PCR reactions using 1×AMPure PB beads, as described by the manufacturer (part no. 100-265-900, Pacific Biosciences). PIK3CA amplicons were visualized and quantified using the 2100 Bioanalyzer System with the DNA 12000 kit (Agilent Biosciences).

SMRTbell Library Preparation and Sequencing

SMRTbell template libraries of the ˜3.3-kb PIK3CA amplicon insert size were prepared according to the manufacturer's instructions using the SMRTbell Template Prep Kit 1.0 (part no. 100-259-100; Pacific Biosciences). A total of 250 ng of purified PIK3CA amplicon was added directly into the DNA damage repair step of the Amplicon Template Preparation and Sequencing protocol. Library quality and quantity were assessed using the DNA 12000 Kit and the 2100 Bioanalyzer System (Agilent), as well as the Qubit dsDNA Broad Range Assay kit and Qubit Fluorometer (Thermo Fisher). Sequencing primer annealing and P6 polymerase binding were performed using the recommended 20:1 primer:template ratio and 10:1 polymerase:template ratio, respectively. SMRT sequencing was performed on the PacBio RS II using the C4 sequencing kit with magnetic bead loading and one-cell-per-well protocol and 240-minute movies.

SMRT-Seq Haplotype Generation and Variant Calling

In order to generate haplotypes and identify variants, data were processed by the Minor Variants Analysis Tool as part of the SMRTLink 5.1 bioinformatics suite (Pacific Biosciences). Briefly, circular consensus sequence (CCS) reads were generated and filtered on reads that were ≥99.9% (Q30) accurate as input for haplotype and variant analysis. A conservative 5% variant frequency threshold was also applied, such that the phased haplotypes were generated using variants called with very high confidence. Phased haplotypes indicated those variants that were present in cis- or trans- within each selected sample.

Mutagenesis and Cloning

For pBabe puro HA PIK3CA and pcDNA 3.4-PIK3CA, the SNP coding for I143V was mutated back to the wildtype isoleucine by site-directed mutagenesis. For pBabe puro HA PIK3CA, the N-terminal HA tag was deleted by site-directed mutagenesis. For pDONR223_PIK3CA_WT, a C-terminal stop codon was inserted by site-directed mutagenesis. In total, all of these modifications resulted in untagged wildtype PIK3CA in the various plasmids. Onto these wildtype backbones, E545K and H1047R mutants were cloned. After this first round of mutagenesis, E453Q, E726K, and M1043L were cloned into the E545K and H1047R plasmids to create dual compound mutants. pDONR plasmids were recombined with the pLX-302 acceptor plasmid using Gateway LR Clonase II Enzyme mix (Thermo Fisher).

Cell Lines, Retroviral, and Lentiviral Production, and Drugs

NIH-3T3 cells were maintained in DMEM media supplemented with 10% FCS and 1% Pen/Strep. MCF-10A cells were maintained in DF-12 media supplemented with 5% filtered horse serum (Invitrogen), EGF (20 ng/μL) (Sigma), hydrocortisone (0.5 mg/mL) (Sigma), cholera toxin (100 mg/mL) (Sigma), insulin (10m/mL) (Sigma), and 1% penicillin/streptomycin. MCF7 cells and 293T cells were maintained in DMEM media supplemented with 10% FBS and 1% Pen/Strep. Cells were used at low passages and were incubated at 37° C. in 5% CO2.

For retroviral and lentiviral production, 7×10⁶ 293 T cells were seeded in 10-cm plates, transfected with the plasmid of interest, pCMV-VSVG, and pCMV-dR8.2 (for lentivirus) using Jetprime (Polyplus Transfection). Viruses were harvested 48 hours after transfection and were filtered through a 0.45 μm filter (Millipore). Target cells were infected using fresh viral supernatants and were selected using puromycin (2 μg/mL) to obtain stable clones. Cell lines were genotyped to confirm the presence of the PIK3CA cDNA sequence.

Cell Proliferation Assays

MCF10A cell lines were seeded in serum starved media (MCF10A media without EGF or insulin), at 10000 cells/mL in 12 well plates. Cells were grown and time points were collected daily from 0-4 days and fixed in formalin. Formalin fixed cells were developed using crystal violet and pictures were taken for day 4 growth. Acetic acid was added and OD595 was obtained. OD values were normalized to day 0 for each cell lines and plotted.

Western Blotting

MCF10A, NIH-3T3 cells, and MCF7 cells were seeded in normal growth medium, either 4 million cells in 10 cm dishes or 400000 cells in 6 cm plates. 24 hours later, cells were washed twice with PBS then refreshed with serum starved media. Serum starved media for MCF10A cells used MCF10A media with 5% horse serum and without EGF or insulin. Serum starved media for NIH-3T3 and MCF7 cells used 0.1% FCS and 0.1% FBS, respectively. For drugging experiments cells were washed twice with PBS then refreshed with serum starved media with DMSO or 1 μM BYL719. 24 hours later, Cells were washed with PBS twice, and lysed in RIPA buffer supplemented with protease and phosphatase inhibitors (Roche). Xenograft tumor samples were also lysed in RIPA buffer supplemented with protease and phosphatase inhibitors. Protein extracts were quantified and normalized (NuPage), separated using SDS-PAGE gells, and transferred to PVDF membranes. Membranes were probed using specific antibodies. p110α, pAKT (S473), pAKT (T308), total AKT, pPRAS40, pS6 (240/4), pS6 (235/6), total S6, pERK1/2 (T202/Y204), total ERK, and vinculin were purchased from Cell Signaling Technology (CST). All primary antibodies were diluted 1:1000 and anti-rabbit IgG secondary antibody (GE Healthcare) (1:4000) was used.

Mouse xenografts 5×10⁶ NIH-3T3 cells in 1:1 PBS/Matrigel (Corning) were injected subcutaneously into six-week-old female athymic nude mice. When tumors reached a volume of ˜150 mm³, mice measured twice a week during a month. 4 tumors per group were used in these studies. For statistical analysis, outliers were removed using Grubbs' test (α=0.05). Tumors were harvested at the end of the experiment, fixed in 4% formaldehyde in PBS, and paraffin-embedded. IHC was performed on a BOND RX processor platform (Leica) using standard protocols with BOND Epitope Retrieval Solution 2 (Leica). Primary staining with pAKT (S473) (D9E), 1:100 (CST) for 30 minutes was followed by staining with a Bond Polymer Refine Detection kit (Leica) for 60 minutes.

Atomic Modeling

The structure of a truncated PI3K complex (PDB 4OVU) (Echeverria, FEBS J, 2015, 282, 3528-3542) of the tagged full length p110α and niSH2 domains of p85α was modeled using PyMOL.

Protein Expression and Purification

EXPI-293F cells (Thermo Fisher) were incubated at 37° C. in 8% CO2, in spinner flasks on an orbital shaker at 125 rpm in Expi293 Expression Medium (Thermo Fisher). 300 ug of pcDNA 3.4-PIK3CA and 200 ug pcDNA 3.4-PIK3R1 were combined and diluted in Opti-MEM I Reduced Serum Medium (Thermo Fisher). ExpiFectamine 293 Reagent (Thermo Fisher) was diluted with Opti-MEM separately then combined with diluted plasmid DNA for 10 minutes at room temperature. The mixture was then transferred slowly to 500 mL EXPI-293F cells (3×10⁶ cells/mL) and incubated. 24 hours later, ExpiFectamine 293 Transfection Enhancer 1 and Enhancer 2 (Thermo Fisher) were added. Cells were harvested 3 days after transfection and centrifuged at 4000 rpm for 30 minutes and frozen at −20° C.

All steps of protein purification were performed at 4° C. Cell pellets were solubilized in lysis buffer (50 mM Tris pH 8.0, 400 mM NaCl, 2 mM MgCl₂, 5% glycerol, 1% Triton X-100, 5 mM β-mercaptoethanol, 20 mM imidazole) supplemented with EDTA-free protease inhibitor (Sigma) and lysed using a Dounce homogenizer for 20 strokes. Lysates were centrifuged at 14000 rpm for 60 minutes and clarified lysates were affinity purified on Ni-NTA resin (Qiagen) by batch binding at 4° C. for 1 hour. Resin was washed with 10 column volumes of lysis buffer (50 mM Tris pH 8.0, 500 mM NaCl, 2 mM MgCl₂, 2% glycerol, 20 mM imidazole) and eluted in 10 column volumes of elution buffer (50 mM Tris pH 8.0, 100 mM NaCl, 2 mM MgCl₂, 2% glycerol, 1 mM TCEP, 250 mM imidazole). Eluted protein was buffer exchanged with elution buffer without imidazole, concentrated using 100 kDa Ultra Centrifugal Filter Units (Amicon), and flash frozen in liquid nitrogen with 20% glycerol. Concentrations of PI3K complexes used in all biochemistry experiments were normalized by Western blotting for p110α as compared to 1 μg WT PI3K complex.

Thermal Shift Assays

1 μg of PI3K complex was added to 10 μL 5× Assay Buffer I (SignalChem), 2 μL 1 mM ATP, and 1 μL BSA (2 mg/mL) and distilled water to a total volume of 50 μL into each tube of a MicroAmp Optical 8-Cap strip (Thermo Fisher) at room temperature. For each experiment, one 8-cap strip was prepared per PI3K construct. Tubes were placed in a C1000 Touch Thermocycler (BioRad). Samples were cycled at 46° C. for 30 seconds, then on a temperature gradient from 46°−63° C. for 3 minutes, then 25° C. for 3 minutes. Samples were spun in a minispin centrifuge for 30 seconds and 40 μL of the supernatant was transferred to separate Eppendorf tubes. Tubes were centrifuged at 15000 rpm for 20 minutes at 4° C. 30 μL of the supernatant was transferred to separate Eppendorf tubes with SDS buffer. Samples were loaded and amount of soluble p110α probed by Western blotting across the temperature gradient.

Liposome Preparation and Liposome Binding Assays

PS, PE, and PI were purchased (Avanti) and cholesterol was purchased (Nu Chek Prep). Neutral lipid stocks were prepared at 10 mg/mL in HPLC-grade chloroform from using molar percentages of 35% PE, 25% PS, 5% PI, and 35% cholesterol. PIP2 lipid stocks were prepared at 35% PE, 25% PS, 4.9% PI, 0.1% PIP2, and 35% cholesterol. A gentle stream of argon gas was applied for 15 seconds and tubes were frozen and stored at −20° C. Prior to experiments, the lipid stocks were vortexed and 100 μL of chloroform (HPLC-grade) was transferred to a clean glass vial. Argon gas was immediately applied to the stock tube, capped, and stored at −20° C. Argon gas was applied the 100 μL aliquot leaving a translucent lipid film. 2 mL of 1× filter-sterilized TBSM buffer (50 mM Tris pH 8.0, 50 mM NaCl, 5 mM MgCl₂) was added and lipids were hydrated at room temperature for 1 hour. Liposomes were extruded using a Mini-Extruder kit (Avanti) through an 8.0 μm membrane 15 times. Liposomes were transferred to a clean Eppendorf tube and centrifuged at 15000 rpm for 8 minutes. Supernatant was discarded, and the lipid pellet was resuspended in 100 μL TBSM buffer vigorously until resuspended. 900 μL of TBSM was added for a final volume of 1 mL.

Liposome binding assays were performed at room temperature. 1 μg of PI3K complex in PBS was added to 70 μL liposomes (10 mg/mL) in a total volume of 100 μL. Binding reactions proceeded for 30 minutes. Solutions were centrifuged at 15000 rpm for 15 minutes and supernatant was removed by aspiration. Lipid pellets were mixed with 50 μL SDS buffer, and the amount of bound p110α was probed by Western blotting.

Lipid Kinase Assays

For triplicate kinase reactions, radioactive ATP buffer, protein, and PIP2 master mixes were assembled. The radioactive ATP buffer master mix contained 1100 μL 5× Assay Buffer I (SignalChem), 55 μL ATP (10 mM), 55 μL BSA (2 mg/mL), 55 μL ³²P-labeled ATP (0.01 mCi/uL), and 2805 μL distilled water. The protein master mix contained 4 μg PI3K complex in 16 μL total volume. The PIP2 master mix contained 50 μL PIP2 (Avanti) and 450 μL distilled water. For each construct, 296 μL buffer master mix was combined with 14 μL protein master mix (buffer+protein master mix) and was mixed well by pipetting. 90 μL of the buffer+protein master mix was aliquoted in triplicate, corresponding to a total amount of 1.016 μg PI3K complex per reaction. To this was added 10 μL of PIP2 master mix (100 uL total volume per reaction) and the solution was mixed well by pipetting to start the reaction. Kinase reactions proceeded at 30° C. for 10 minutes. 50 μL of 4N HCL was added to quench the reaction followed by 100 μL of 1:1 methanol-chloroform. Tubes were vortexed for 30 seconds each and centrifuged at 15000 rpm for 10 minutes. Using gel loading pipet tips pipetted with chloroform in and out, 20 μL of the bottom hydrophobic phase was removed and spotted onto a TLC plate (EMD Millipore, M1164870001). Plates were placed in a sealed chamber with 65:35 1-propanol and 2M acetic acid and TLC was run overnight. Plates were exposed to a phosphor screen for 4 hours and imaged on a Typhoon FLA 7000.

Cell Inhibition by PI3K Inhibitors.

1000 MCF10A cells were seeded in 100 μL of MCF10A media (containing 2% horse serum) lacking EGF or insulin, per well, in a 96-well plate. 24 hours later, serial concentrations of BYL719 or GDC-0077 were added in 100 μL of MCF10A media (containing 2% horse serum) lacking EGF or insulin. Cells were incubated for 4 days and then developed with CellTiter-Glo (Promega). Fold inhibition was calculated relative to cell growth in medium without drug.

Clinical Trial Analysis

Progression-free survival analysis was performed on patients enrolled in NCT01870505, a phase I clinical trial of BYL719 (alpelisib) plus letrozole or exemestane for patients (n=51) with hormone-receptor positive locally-advanced unresectable or metastatic breast cancer. 44/51 patients were analyzed for NGS of their tumors (MSK-IMPACT) and/or NGS of their ctDNA (Guardant) and were included in the analysis. Progression free survival was calculated and was compared between dual and single mutant patients. Clinical benefit rates (complete response, partial response or stable disease) were calculated and were compared between dual and single mutant patients using Fisher's exact test.

Oligonucleotides
SMRT-seq primers
Forward:
[Seq ID No: 1]
TGGGACCCGATGCGGTTA
Reverse:
[Seq ID No: 2]
AATCGGTCTTTGCCTGCTGA
E545K
Forward:
[Seq ID No: 3]
CCTCTCTCTGAAATCACTAAGCAGGAGAAAGATTTTC
Reverse:
[Seq ID No: 4]
GAAAATCTTTCTCCTGCTTAGTGATTTCAGAGAGAGG
H1047R
Forward:
[Seq ID No: 5]
CAAATGAATGATGCACGTCATGGTGGCTGGAC
Reverse:
[Seq ID No: 6]
GTCCAGCCACCATGACGTGCATCATTCATTTG
E453Q
Forward:
[Seq ID No: 7]
CCAGTACCTCATGGATTACAGGATTTGCTGAACCCTATTG
Reverse:
[Seq ID No: 8]
CAATAGGGTTCAGCAAATCCTGTAATCCATGAGGTACTGG
E726K
Forward:
[Seq ID No: 9]
GAGAAGAAGGATAAAACACAAAAGGTAC
Reverse:
[Seq ID No: 10]
GTACCTTTTGTGTTTTATCCTTCTTCTC
M1043L
Forward:
[Seq ID No: 11]
GTATTTCATGAAACAACTGAATG
ATGCACATCATGGTGGCTGGAC
Reverse:
[Seq ID No: 12]
GTCCAGCCACCATGATGTGCATCATT
CAGTTGTTTCATGAAATAC
Mutate in C-terminal stop codon for WT
in pDONR223
Forward:
[Seq ID No: 13]
CATGCATTGAACTGATTGCCAACTTTC
Reverse:
[Seq ID No: 14]
GAAAGTTGGCAATCAGTTCAATGCATG
Mutate out N-terminal HA tag
(in pBabe puro Myr HA PIK3CA)
Forward:
[Seq ID No: 15]
GATCCAAGCTTCACCATGCCTCCAAGACCATCATCA
Reverse:
[Seq ID No: 16]
TGATGATGGTCTTGGAGGCATGGTGAAGCTTGGATC
I143 (mutating back to WT I143 in pBabe
puro Myr HA PIK3CA which has I143V SNP)
Forward:
[Seq ID No: 17]
GACTTCCGAAGAAATATTCTGAACGTTTGTAAA
Reverse:
[Seq ID No: 18]
TTTACAAACGTTCAGAATATTTCTTCGGAAGTC

### Example 3: Multiple PIK3CA Mutant Tumors are Hypersensitive to PI3K Inhibition in Patients

PIK3CA is the most frequently mutated oncogene across all human cancers, and codes for p110α, the catalytic subunit of the phosphoinositide 3-kinase alpha (PI3Kα) complex, which is necessary for normal growth and proliferation (Bailey et al., Cell 174, 1034-1035 (2018); Whitman et al., Nature 332, 644-646 (1988)). PI3Kα is comprised of p110α and the regulatory subunit p85α, which catalyzes the phosphorylation of the lipid phosphatidylinositol 4,5 bisphosphate (PIP2) to phosphatidylinositol 3,4,5-trisphosphate (PIP3), which in turn initiates a downstream signaling cascade involving the activation of AKT and mammalian target of rapamycin (mTOR) (Fruman et al., Cell 170, 605-635 (2017)). PI3Kα is activated by binding to membrane-bound receptor tyrosine kinases (RTKs) and can be constitutively activated by oncogenic mutations. Many distinct cancer associated PIK3CA mutations have been identified including hotspot single amino acid substitutions in the helical (E542K/E545K) or kinase (H1047R) domains Samuel et al., Science 304, 554 (2004)). These mutations are considered oncogenic in multiple cancer histologies including breast cancer, where PIK3CA mutations are present in 40% of estrogen receptor-positive (ER+), human epidermal growth factor receptor 2-negative (HER2-) primary and metastatic tumors and have been proposed as a target for cancer therapy (Samuels et al., Cancer Cell 7, 561-573 (2005); Kang et al., Proc Natl Acad Sci USA 102, 802-807 (2005); Zhao et al., Cancer Cell 3, 483-495 (2003); Razavi et al., Cancer Cell 34, 427-438 e426 (2018)).

Based on this hypothesis, several PI3K inhibitors have been studied, with clinical activity in patients with PIK3CA-mutant breast cancer, although toxicities were significant and precluded their clinical development (Baselga et al., Lancet Oncol 18, 904-916 (2017); Di Leo et al., Lancet Oncol 19, 87-100 (2018); Baselga et al, Journal of Clinical Oncology, 2018, 36, no. 18_suppl.). More recently, a more selective PI3Kα inhibitor alpelisib has shown improved tolerability and a large randomized phase 3 clinical trial study has shown improved progression-free survival (PFS) in patients with ER+PIK3CA mutant metastatic breast cancer (Mayer et al., Clin Cancer Res 23, 26-34 (2017); Azambuia et al., Journal of Clinical Oncology 33, no. 15 suppl; Andre et al., N Engl J Med 380, 1929-1940 (2019)).

As with other targeted therapies in cancer, acquired and adaptive resistance mechanisms limit the efficacy of PI3Kα inhibitors. On the other hand, in early clinical trials, it was observed that there was also a population of patients that displayed deep and prolonged clinical benefit (Juric et al., Nature 518, 240-244 (2015); Bosch et al., Sci Transl Med 7, 283ra251 (2015); Toska et al., Science 355, 1324-1330 (2017); Hopkins et al., Nature 560, 499-503 (2018); Juric et al., Clin Oncol 36, 1291-1299 (2018)). In search of genomic signals of improved clinical response to PI3K inhibitors, the present disclosure identified double PIK3CA mutations as a candidate biomarker. This finding prompted to undertake a comprehensive analysis of the prevalence of these mutations and to investigate their potential biological relevance and correlation with sensitivity to PI3Kα inhibitors.

Durable Responses to Alpelisib in Some Patients with Double PIK3CA Mutant Breast Cancer

The present disclosure previously reported an exceptional responder breast cancer patient to alpelisib monotherapy, who eventually developed acquired resistance through convergent

PTEN mutations. In this patient, also it was detected the presence of double PIK3CA mutations in all metastatic sites and at different times over tumor evolution, with equal variant allele frequencies (VAFs) of both mutations (FIG. 18B). The present disclosure analyzed data from a phase 1 clinical trial (n=51) investigating alpelisib with an aromatase inhibitor in heavily pre-treated patients with ER+ metastatic breast cancer. PIK3CA mutational status was determined by tumor NGS. Patients with double PIK3CA mutant tumors had a longer median PFS than patients with single mutant tumors or WT tumors but this was not statistically significant due to small numbers (FIG. 18C). The present disclosure hypothesized that this sensitivity was due the PI3K inhibitor rather than the hormonal therapy, as patients with double PIK3CA mutant tumors do not have improved PFS when treated with aromatase inhibition or fulvestrant alone as compared to patients with single mutant or WT tumors on retrospective analysis (FIG. 18A).

### Double PIK3CA Mutant Tumors are Frequent in Breast Cancer and Other Tumor Histologies

The present disclosure analyzed a publicly available cohort (n=70754) across different cancer histologies from cBioPortal (Cerami et al., Cancer Discov 2, 401-404 (2012); Gao et al., Sci Signal 6, pl1 (2013)) and identified 4526 PIK3CA mutant tumors, 576 (13%) of which contain multiple PIK3CA mutations (FIG. 9A, Table 4). The present disclosure recapitulated these findings using a cohort enriched with metastatic tumors (n=28139) across different cancer histologies, sequenced by MSK-IMPACT (Cheng et al., J Mol Diagn 17, 251-264 (2015)). The present disclosure identified 3740 PIK3CA mutant tumors, 451 (12%) of which contain multiple PIK3CA mutations (FIG. 10F, Table 5). In both cBioPortal and MSK-IMPACT cohorts, breast, uterine, and colorectal cancers had the greatest number of multiple PIK3CA mutant tumors. The present disclosure also analyzed individual breast cancer subsets of the cBioPortal dataset and found similar frequencies of multiple PIK3CA mutant breast cancer in METABRIC (13%), TCGA (11%), and other data sets (8%) (FIG. 10B) (Curtis et al., Nature 486, 346-352 (2012); N. Cancer Genome Atlas, Nature 490, 61-70 (2012); Banerji et al., Nature 486, 400-404 (2012); Wagle et al., Cancer Research 78, 5371-5371 (2018)). The vast majority of multiple PIK3CA mutant tumors in all these patient cohorts carried exactly two mutations (FIG. 10C).

TABLE 4
List of multiple PIK3CA mutant tumors (n = 576) (cBioPortal)

| | | Protein | Protein | Protein | Protein | Protein | Protein |
|---|---|---|---|---|---|---|---|
| Sample ID | Cancer Type | Change 1 | Change 2 | Change 3 | Change_4 | Change_5 | Change 6 |
| AMPAC_2713 | Ampullary | Y1021C | V146I | | | | |
| | Carcinoma | | | | | | |
| P-0011521-T01-IM5 | Anaplastic | E542K | G106V | | | | |
| | Astrocytoma | | | | | | |
| P-0007319-T01-IM5 | Basaloid Penile | E545G | M1055I | R617W | | | |
| | Squamous Cell | | | | | | |
| | Carcinoma | | | | | | |
| B52 | Bladder combined | E726K | G451R | | | | |
| B98 | Bladder combined | E542K | E545Q | | | | |
| BL033 | Bladder combined | E545K | E542K | | | | |
| DS-sig-003-P | Bladder combined | M1043I | D1017H | E1012Q | | | |
| P-0000043-T02-IM3 | Bladder combined | E545K | V356A | | | | |
| P-0000423-T01-IM3 | Bladder combined | E545K | E453Q | | | | |
| P-0002659-T01-IM3 | Bladder combined | E545K | Q682H | | | | |
| P-0004424-T01-IM5 | Bladder combined | E542K | H1047R | | | | |
| P-0009101-T01-IM5 | Bladder combined | E545K | E542K | | | | |
| P-0010921-T01-IM5 | Bladder combined | E542K | E453Q | H1065Y | | | |
| TCGA-2F-A9KR-01 | Bladder combined | E542K | E1012Q | | | | |
| TCGA-4Z-AA7Y-01 | Bladder combined | E545G | E545K | | | | |
| TCGA-4Z-AA89-01 | Bladder combined | N345K | R93Q | | | | |
| TCGA-5N-A9KI-01 | Bladder combined | E545K | R274K | | | | |
| TCGA-FD-A5BX-01 | Bladder combined | E365K | S66C | | | | |
| TCGA-UY-A78P-01 | Bladder combined | E418K | M1043I | | | | |
| TCGA-XF-AAMG-01 | Bladder combined | E542K | W552C | | | | |
| TCGA-ZF-A9R2-01 | Bladder combined | E726K | E710Q | | | | |
| TCGA-ZF-A9RG-01 | Bladder combined | E545K | E453K | | | | |
| P-0004635-T01-IM5 | Breast combined | H1047Y | C420R | | | | |
| P-0000075-T01-IM3 | Breast combined | H1047R | N142K | | | | |
| P-0000107-T01-IM3 | Breast combined | Y1021H | D1017E | I1019V | | | |
| P-0000138-T01-IM3 | Breast combined | E542K | E453K | | | | |
| P-0000138-T02-IM3 | Breast combined | E542K | E453K | | | | |
| P-0000155-T01-IM3 | Breast combined | H1047R | D350N | | | | |
| P-0000167-T01-IM3 | Breast combined | K111E | E418K | | | | |
| P-0000167-T02-IM3 | Breast combined | E542K | K111E | | | | |
| P-0000207-T01-IM3 | Breast combined | K111E | N345K | | | | |
| P-0000234-T01-IM3 | Breast combined | E542K | M1043I | | | | |
| P-0000234-T02-IM5 | Breast combined | E542K | M1043I | | | | |
| P-0000356-T01-IM3 | Breast combined | H1047R | E453K | | | | |
| P-0000381-T01-IM3 | Breast combined | H1047R | E726K | | | | |
| P-0000397-T01-IM3 | Breast combined | E542K | E453K | | | | |
| P-0000592-T01-IM3 | Breast combined | E545K | M1043I | | | | |
| P-0000607-T01-IM3 | Breast combined | H1047L | E545Q | | | | |
| P-0001043-T01-IM3 | Breast combined | H1047R | R93L | | | | |
| P-0001114-T02-IM3 | Breast combined | E545K | E453Q | E978Q | | | |
| P-0001351-T01-IM3 | Breast combined | E545K | E726K | | | | |
| P-0001351-T02-IM5 | Breast combined | E545K | E726K | | | | |
| P-0001631-T01-IM3 | Breast combined | E545K | M1043L | | | | |
| P-0001631-T02-IM5 | Breast combined | E545K | M1043L | | | | |
| P-0001902-T01-IM3 | Breast combined | E542K | E545D | | | | |
| P-0001990-T01-IM3 | Breast combined | H1047Y | N345K | | | | |
| P-0002124-T01-IM3 | Breast combined | H1047R | E365K | | | | |
| P-0002562-T01-IM3 | Breast combined | E545K | E453Q | | | | |
| P-0002562-T02-IM5 | Breast combined | E545K | E453Q | | | | |
| P-0002657-T01-IM3 | Breast combined | G118D | G364R | | | | |
| P-0002667-T01-IM3 | Breast combined | H1047R | N107I | | | | |
| P-0002756-T02-IM5 | Breast combined | E545K | E453Q | | | | |
| P-0002841-T01-IM3 | Breast combined | H1047R | K111N | | | | |
| P-0002841-T02-IM6 | Breast combined | H1047R | K111N | | | | |
| P-0002922-T01-IM3 | Breast combined | H1047R | E545Q | | | | |
| P-0003224-T01-IM5 | Breast combined | H1047R | G106V | | | | |
| P-0003987-T01-IM5 | Breast combined | E545K | H1047R | | | | |
| P-0004187-T01-IM5 | Breast combined | H1047R | E970K | | | | |
| p-0004194-T01-IM5 | Breast combined | E545K | D725G | | | | |
| P-0004196-T01-IM5 | Breast combined | H1047R | Q958K | | | | |
| P-0004264-T01-IM5 | Breast combined | H1047R | E726K | | | | |
| P-0004433-T01-IM5 | Breast combined | E545K | L252F | | | | |
| P-0004913-T01-IM5 | Breast combined | H1047R | E542A | | | | |
| P-0005032-T01-IM5 | Breast combined | L455Wfs*6 | L452Qfs*5 | | | | |
| P-0005037-T01-IM5 | Breast combined | N345K | R93Q | | | | |
| P-0005120-T01-IM5 | Breast combined | E542K | E726K | | | | |
| P-0005154-T01-IM5 | Breast combined | E542K | H1047R | | | | |
| P-0005220-T01-IM5 | Breast combined | H1047R | C407F | | | | |
| P-0005242-T01-IM5 | Breast combined | E545K | E726K | | | | |
| P-0005314-T01-IM5 | Breast combined | E542K | E726K | | | | |
| P-0005611-T01-IM5 | Breast combined | M1043I | E39K | | | | |
| P-0005818-T01-IM5 | Breast combined | H1047R | G118D | | | | |
| P-0005968-T01-IM5 | Breast combined | R88Q | H1047R | | | | |
| P-0006161-T03-IM5 | Breast combined | E542K | E453K | | | | |
| P-0006166-T01-IM5 | Breast combined | E542K | E726K | | | | |
| P-0006335-T01-IM5 | Breast combined | E545K | T1025A | | | | |
| P-0006660-T01-IM5 | Breast combined | R88Q | Q546H | | | | |
| P-0006723-T01-IM5 | Breast combined | H1047R | E81K | | | | |
| P-0006780-T01-IM5 | Breast combined | E726K | V344M | | | | |
| P-0006787-T01-IM5 | Breast combined | H1047R | E726K | | | | |
| P-0007396-T01-IM5 | Breast combined | N345K | E970K | | | | |
| P-0008679-T01-IM5 | Breast combined | H1047R | N345I | | | | |
| P-0008679-T03-IM5 | Breast combined | H1047R | N345I | | | | |
| P-0008694-T01-IM5 | Breast combined | E542K | E453K | | | | |
| P-0008845-T01-IM5 | Breast combined | K111E | G118D | | | | |
| P-0009745-T02-IM6 | Breast combined | C420R | E726K | | | | |
| P-0010002-T01-IM5 | Breast combined | G1049R | Q546P | | | | |
| P-0010043-T01-IM5 | Breast combined | H1047R | P539R | | | | |
| P-0010703-T01-IM5 | Breast combined | H1047L | E726K | | | | |
| P-0010917-T01-IM5 | Breast combined | E542K | E726K | | | | |
| P-0011305-T01-IM5 | Breast combined | E545K | E726K | | | | |
| P-0011355-T01-IM5 | Breast combined | H1047R | E81K | | | | |
| P-0011420-T01-IM5 | Breast combined | E545K | A1066V | | | | |
| P-0012911-T01-IM5 | Breast combined | C420R | R108H | | | | |
| P-0013491-T01-IM5 | Breast combined | G118D | E542Q | | | | |
| P-0013771-T01-IM5 | Breast combined | H1047R | E726K | | | | |
| P-0013895-T01-IM5 | Breast combined | E542K | E726K | | | | |
| P-0014091-T01-IM5 | Breast combined | H1047L | E81K | | | | |
| P-0014136-T01-IM5 | Breast combined | E542K | N457K | | | | |
| P-0014278-T01-IM6 | Breast combined | E542K | E726K | | | | |
| P-0014479-T01-IM6 | Breast combined | E545K | E542Q | | | | |
| P-0014480-T01-IM6 | Breast combined | E542K | E726K | | | | |
| P-0014515-T01-IM6 | Breast combined | Q546R | M1004I | | | | |
| P-0014622-T01-IM6 | Breast combined | H1047R | P104L | | | | |
| P-0014737-T01-IM6 | Breast combined | H1047L | P539R | | | | |
| P-0014860-T01-IM6 | Breast combined | H1047L | E545D | | | | |
| P-0014940-T01-IM6 | Breast combined | E542K | E726K | | | | |
| P-0014943-T01-IM6 | Breast combined | H1047R | E542G | | | | |
| P-0015005-T01-IM6 | Breast combined | E545K | Q546R | | | | |
| P-0015097-T01-IM6 | Breast combined | H1047R | E453K | | | | |
| P-0015499-T01-IM6 | Breast combined | H1047R | E418K | | | | |
| P-0015633-T01-IM6 | Breast combined | H1047L | I112F | | | | |
| P-0015640-T01-IM6 | Breast combined | N1044K | D350N | | | | |
| P-0015944-T01-IM6 | Breast combined | C420R | E726K | | | | |
| P-0015964-T01-IM6 | Breast combined | E545K | M1004I | | | | |
| P-0016773-T01-IM6 | Breast combined | G106V | Q546H | | | | |
| P-0016786-T01-IM6 | Breast combined | E545K | M1043I | | | | |
| P-0016802-T01-IM6 | Breast combined | H1047R | G118D | | | | |
| P-0016840-T01-IM6 | Breast combined | H1047R | V344M | | | | |
| P-0017581-T01-IM5 | Breast combined | E545G | T1025A | | | | |
| P-0017818-T01-IM6 | Breast combined | E545K | H1048R | | | | |
| P-0018891-T01-IM6 | Breast combined | H1047R | N107Y | | | | |
| TCGA-D8-A1JS-01 | Breast combined | H1047R | V344M | | | | |
| TCGA-LD-A74U-01 | Breast combined | E545K | C420R | | | | |
| BR-M-150 | Breast combined | E545G | E453K | | | | |
| MB-0014 | Breast combined | G1049R | Q546H | | | | |
| MB-0162 | Breast combined | L452Kfs*4 | E453Dfs*7 | | | | |
| MB-0172 | Breast combined | E545K | H1047R | | | | |
| MB-0261 | Breast combined | H1047R | E453K | | | | |
| MB-0345 | Breast combined | H1047R | R108H | | | | |
| MB-0388 | Breast combined | E545K | S509Y | | | | |
| MB-0393 | Breast combined | E545K | G914R | | | | |
| MB-0399 | Breast combined | H1047R | L10_P17del | | | | |
| MB-0463 | Breast combined | E545K | E726K | | | | |
| MB-0475 | Breast combined | H1047R | E81K | | | | |
| MB-0554 | Breast combined | H1047R | H1048R | D1029H | | | |
| MB-0571 | Breast combined | N345K | N1044K | | | | |
| MB-0598 | Breast combined | V105del | K148N | | | | |
| MB-0623 | Breast combined | H1047R | T727K | | | | |
| MB-0632 | Breast combined | H1047R | E80K | | | | |
| MB-0904 | Breast combined | E542K | T727K | | | | |
| MB-0906 | Breast combined | H1047R | G118D | | | | |
| MB-2617 | Breast combined | H1047R | P471L | | | | |
| MB-2944 | Breast combined | Q546R | E453K | | | | |
| MB-2971 | Breast combined | E542K | Y1021H | | | | |
| MB-3254 | Breast combined | H1047R | E453K | | | | |
| MB-3295 | Breast combined | H1047R | E365K | | | | |
| MB-3344 | Breast combined | H1047R | E453K | | | | |
| MB-3824 | Breast combined | H1047L | P449T | | | | |
| MB-4343 | Breast combined | H1047R | H1048R | | | | |
| MB-4362 | Breast combined | E542K | E970K | | | | |
| MB-4607 | Breast combined | N345K | F83C | | | | |
| MB-4739 | Breast combined | E545K | P104R | | | | |
| MB-4746 | Breast combined | E542K | M1043I | | | | |
| MB-4749 | Breast combined | Y1021H | V105del | | | | |
| MB-4791 | Breast combined | M1043I | E970K | | | | |
| MB-4800 | Breast combined | L113Sfs*32 | K111Dfs*16 | | | | |
| MB-4801 | Breast combined | H450_P458del | H1065Y | Q1064H | | | |
| MB-4843 | Breast combined | H1047R | E453Q | E726K | | | |
| MB-4869 | Breast combined | H1047R | P471L | R108H | | | |
| MB-4959 | Breast combined | Q546P | *1069Wext*4 | | | | |
| MB-4961 | Breast combined | H1047R | E726K | | | | |
| MB-4967 | Breast combined | H1047R | E81K | | | | |
| MB-4969 | Breast combined | E545K | E726K | | | | |
| MB-5072 | Breast combined | H1047R | E542A | | | | |
| MB-5088 | Breast combined | H1047L | C420R | | | | |
| MB-5119 | Breast combined | E726K | N1044K | | | | |
| MB-5134 | Breast combined | H1047Y | N345K | | | | |
| MB-5169 | Breast combined | E542K | E726K | | | | |
| MB-5179 | Breast combined | H1047R | I459T | | | | |
| MB-5182 | Breast combined | H1047R | I354F | | | | |
| MB-5196 | Breast combined | E545K | E542K | | | | |
| MB-5270 | Breast combined | N345K | N114S | | | | |
| MB-5275 | Breast combined | E545K | D1017H | | | | |
| MB-5288 | Breast combined | E545K | M1043I | | | | |
| MB-5401 | Breast combined | G118D | T957P | | | | |
| MB-5425 | Breast combined | H1047R | R108H | | | | |
| MB-5446 | Breast combined | R88Q | C420R | | | | |
| MB-5470 | Breast combined | H1047R | G106R | | | | |
| MB-5482 | Breast combined | H1047R | E365K | | | | |
| MB-5486 | Breast combined | Q546P | P539R | | | | |
| MB-5511 | Breast combined | H1047R | E81A | | | | |
| MB-5582 | Breast combined | H1047R | P104L | | | | |
| MB-5599 | Breast combined | H1047R | P104R | | | | |
| MB-5623 | Breast combined | E545K | E726K | | | | |
| MB-5656 | Breast combined | E545K | E542K | H1047R | | | |
| MB-6006 | Breast combined | H1047R | E726K | | | | |
| MB-6007 | Breast combined | H1047R | E726K | | | | |
| MB-6018 | Breast combined | H1047R | R88Q | | | | |
| MB-6019 | Breast combined | H1047R | Y1021C | E110del | | | |
| MB-6029 | Breast combined | H1047R | I69N | | | | |
| MB-6030 | Breast combined | H1047R | R93W | | | | |
| MB-6047 | Breast combined | C420R | E970K | | | | |
| MB-6164 | Breast combined | H1047R | H1048R | | | | |
| MB-6194 | Breast combined | H1047R | K111N | | | | |
| MB-6207 | Breast combined | E542K | T1025S | | | | |
| MB-7005 | Breast combined | H1047Y | G118D | | | | |
| MB-7042 | Breast combined | E545K | M1004I | | | | |
| MB-7061 | Breast combined | H1047R | G1007R | | | | |
| MB-7195 | Breast combined | H1047R | G106R | | | | |
| MB-7200 | Breast combined | H1047R | R108H | | | | |
| MB-7230 | Breast combined | N345K | T727K | | | | |
| MB-7244 | Breast combined | H1047R | H1048R | | | | |
| MBC- | Breast combined | E542K | T1025A | | | | |
| MBCProject_27uAugT4- | | | | | | | |
| Tumor-SM-DL45T | | | | | | | |
| MBC- | Breast combined | H1047L | E81K | | | | |
| MBCProject_57iLiJIl- | | | | | | | |
| Tumor-SM-CGLIV | | | | | | | |
| TCGA-3C-AALK-01 | Breast combined | E542K | M1004I | | | | |
| TCGA-A1-A0SI-01 | Breast combined | H1047R | E726K | | | | |
| TCGA-A2-A0CP-01 | Breast combined | H1047R | E726K | | | | |
| TCGA-A2-A0EV-01 | Breast combined | H1047R | E469delinsDK | | | | |
| TCGA-A2-A0T7-01 | Breast combined | E542K | M1043I | | | | |
| TCGA-A8-A075-01 | Breast combined | E545K | E453K | | | | |
| TCGA-A8-A095-01 | Breast combined | H1047L | E726K | | | | |
| TCGA-AC-A23H-01 | Breast combined | D603H | L989V | | | | |
| TCGA-AN-A0XO-01 | Breast combined | E453K | E542K | | | | |
| TCGA-AO-A0JC-01 | Breast combined | R108H | E545K | | | | |
| TCGA-AO-A0JF-01 | Breast combined | E545K | E453K | | | | |
| TCGA-AO-A12A-01 | Breast combined | E545G | E542K | | | | |
| TCGA-AO-A1KR-01 | Breast combined | H1047R | M1004I | | | | |
| TCGA-B6-A0RO-01 | Breast combined | H1047R | P539R | | | | |
| TCGA-BH-A0B6-01 | Breast combined | E453K | E81K | | | | |
| TCGA-BH-A0BT-01 | Breast combined | H1047R | P366R | | | | |
| TCGA-BH-A0DV-01 | Breast combined | E545K | E726K | E726G | | | |
| TCGA-BH-A0W7-01 | Breast combined | H1047R | E726K | | | | |
| TCGA-BH-A18F-01 | Breast combined | H1047R | Q546K | | | | |
| TCGA-BH-A202-01 | Breast combined | H1047R | E365V | | | | |
| TCGA-C8-A131-01 | Breast combined | H1047R | R108H | | | | |
| TCGA-C8-A278-01 | Breast combined | H1047L | M1040V | | | | |
| TCGA-D8-A1JD-01 | Breast combined | E545K | E726K | M1004I | | | |
| TCGA-D8-A1JF-01 | Breast combined | E545K | R398H | | | | |
| TCGA-D8-A1JG-01 | Breast combined | P447_L455del | L456Afs*13 | | | | |
| TCGA-E2-A159-01 | Breast combined | E542K | E542G | | | | |
| TCGA-E2-A1IN-01 | Breast combined | E542K | C901F | | | | |
| TCGA-E9-A1RH-01 | Breast combined | E103_P104del | L531V | | | | |
| TCGA-EW-A1P5-01 | Breast combined | P447_L455del | L456Afs*13 | | | | |
| TCGA-EW-A1PE-01 | Breast combined | Q546K | T1025A | | | | |
| TCGA-GM-A2D9-01 | Breast combined | H1047R | E453K | | | | |
| TCGA-GM-A2DH-01 | Breast combined | Q546R | G1007R | | | | |
| TCGA-OL-A5RX-01 | Breast combined | D939G | P366R | | | | |
| BR-M-083 | Breast combined | H1047R | E542Q | | | | |
| MB-0083 | Breast combined | N345K | E81K | | | | |
| MB-0101 | Breast combined | H1047L | E726K | | | | |
| MB-0188 | Breast combined | E542K | E726K | | | | |
| MB-0306 | Breast combined | H1047R | E726K | | | | |
| MB-0578 | Breast combined | E545K | M1043V | | | | |
| MB-2840 | Breast combined | N345K | M1043I | | | | |
| MB-4484 | Breast combined | H1047R | P471A | | | | |
| MB-4697 | Breast combined | N345K | E726K | | | | |
| MB-4743 | Breast combined | E545K | M1004I | | | | |
| MB-4764 | Breast combined | E542K | E726K | | | | |
| MB-4802 | Breast combined | E81K | D1017H | | | | |
| MB-5035 | Breast combined | E542K | E726K | | | | |
| MB-5107 | Breast combined | K111E | G320A | | | | |
| MB-5326 | Breast combined | K111E | M1043I | | | | |
| MB-6021 | Breast combined | H1047R | N345K | | | | |
| MB-7268 | Breast combined | E545K | E726K | | | | |
| TCGA-A2-A0EN-01 | Breast combined | H1047R | H1065L | | | | |
| TCGA-AC-A5XS-01 | Breast combined | E545G | E545K | C378F | E545R | | |
| TCGA-B6-A0IP-01 | Breast combined | R88Q | H1047R | F83L | | | |
| TCGA-B6-A0RQ-01 | Breast combined | E542G | E545K | | | | |
| TCGA-E2-A10F-01 | Breast combined | H1047R | E726K | | | | |
| TCGA-E2-A14U-01 | Breast combined | E542K | H1047R | | | | |
| TCGA-E2-A2P5-01 | Breast combined | N345K | G914R | | | | |
| TCGA-EW-A1J5-01 | Breast combined | E545K | E726K | | | | |
| TCGA-JL-A3YX-01 | Breast combined | E542K | E726K | | | | |
| TCGA-LQ-A4E4-01 | Breast combined | H1047R | E542G | | | | |
| MB-0171 | Breast combined | E365K | C420R | | | | |
| MB-0353 | Breast combined | H1047R | G118D | | | | |
| MB-0356 | Breast combined | H1047R | H1048R | | | | |
| MB-0379 | Breast combined | E545K | G320A | | | | |
| MB-0504 | Breast combined | H1047R | R108H | | | | |
| MB-0653 | Breast combined | H1047L | E385K | | | | |
| MB-0897 | Breast combined | E542K | E726K | | | | |
| MB-5412 | Breast combined | H1047R | R108H | | | | |
| MB-6022 | Breast combined | E545A | G1050S | | | | |
| MB-0170 | Breast combined | N345K | N1044K | | | | |
| MB-0295 | Breast combined | H1047R | P104L | | | | |
| MB-0363 | Breast combined | E542K | N345K | | | | |
| MB-0891 | Breast combined | H1047R | K111E | | | | |
| MB-2790 | Breast combined | E726K | P449_L452del | | | | |
| MB-3181 | Breast combined | M1043V | E726K | | | | |
| MB-3228 | Breast combined | E545K | D725N | | | | |
| MB-3439 | Breast combined | E542K | D1045N | Q1064H | | | |
| MB-3490 | Breast combined | H1047R | R88Q | | | | |
| MB-3871 | Breast combined | N345K | S161R | | | | |
| MB-7006 | Breast combined | E545K | E542K | E726K | D725N | | |
| MBC_109 | Breast combined | E545K | E726K | | | | |
| MBC_187 | Breast combined | H1047R | P366R | | | | |
| MBC_189 | Breast combined | E542K | E726K | | | | |
| MBC_29 | Breast combined | H1047R | Q546K | | | | |
| MBC_45 | Breast combined | E545K | E726K | | | | |
| MBC_50 | Breast combined | E545K | E385K | | | | |
| MBC_95 | Breast combined | E542K | E453K | | | | |
| MTS-T0035 | Breast combined | H1047R | N345I | | | | |
| MTS-T0065 | Breast combined | H1047R | E263Q | | | | |
| MTS-T0207 | Breast combined | H1047L | N1068K | | | | |
| MTS-T0255 | Breast combined | E542K | H1047R | | | | |
| MTS-T0327 | Breast combined | H1047R | R108H | | | | |
| MTS-T0340 | Breast combined | E545A | E726K | | | | |
| MTS-T0351 | Breast combined | N1044K | E1032Q | D1045H | | | |
| MTS-T0380 | Breast combined | H1047R | P104L | | | | |
| MTS-T0396 | Breast combined | E545K | E726K | | | | |
| MTS-T1284 | Breast combined | E542K | M1043I | | | | |
| MTS-T1800 | Breast combined | E542K | E726K | | | | |
| MTS-T2408 | Breast combined | H1047R | Q958K | | | | |
| MTS-T2413 | Breast combined | E542K | E726K | | | | |
| PD4120a | Breast combined | H1047R | K111N | | | | |
| PD4125a | Breast combined | E81K | E80K | | | | |
| PD4202a | Breast combined | H1047R | P104L | | | | |
| PD4844a | Breast combined | L113_N114del | M1040T | | | | |
| P-0004374-T01-IM5 | Cancer of | E542K | D454G | | | | |
| | Unknown | | | | | | |
| | Primary | | | | | | |
| P-0008153-T01-IM5 | Cancer of | H1047R | R88Q | | | | |
| | Unknown | | | | | | |
| | Primary | | | | | | |
| P-0010527-T01-IM5 | Cervical | E545K | E542K | | | | |
| | combined | | | | | | |
| TCGA-2W-A8YY-01 | Cervical | R88Q | R693H | Y432C | D589N | | |
| | combined | | | | | | |
| TCGA-C5-A1BJ-01 | Cervical | E545Q | E600K | | | | |
| | combined | | | | | | |
| TCGA-C5-A1MH-01 | Cervical | E545K | E726K | | | | |
| | combined | | | | | | |
| TCGA-C5-A1MK-01 | Cervical | E545K | E726K | | | | |
| | combined | | | | | | |
| TCGA-C5-A8XJ-01 | Cervical | E545K | L866F | | | | |
| | combined | | | | | | |
| TCGA-EK-A2RN-01 | Cervical | E545K | E726K | | | | |
| | combined | | | | | | |
| TCGA-FU-A3HZ-01 | Cervical | L339I | E542K | | | | |
| | combined | | | | | | |
| TCGA-VS-A8QA-01 | Cervical | E726K | E453K | | | | |
| | combined | | | | | | |
| TCGA-VS-A959-01 | Cervical | E542K | E453K | | | | |
| | combined | | | | | | |
| TCGA-IR-A3LF-01 | Cervical | E542K | G1007R | | | | |
| | combined | | | | | | |
| TCGA-LP-A7HU-01 | Cervical | E545K | E542K | | | | |
| | combined | | | | | | |
| TCGA-AA-3489-01 | Colorectal | G1049R | D350G | | | | |
| | combined | | | | | | |
| TCGA-AA-3675-01 | Colorectal | H1047Y | V344G | | | | |
| | combined | | | | | | |
| TCGA-AA-3848-01 | Colorectal | R88Q | D350G | | | | |
| | combined | | | | | | |
| TCGA-AA-3977-01 | Colorectal | R88Q | M1043I | | | | |
| | combined | | | | | | |
| TCGA-AA-3984-01 | Colorectal | E970K | R357Q | | | | |
| | combined | | | | | | |
| TCGA-AD-A5EJ-01 | Colorectal | H1047R | E542G | | | | |
| | combined | | | | | | |
| TCGA-AM-5821-01 | Colorectal | H1047R | A224S | | | | |
| | combined | | | | | | |
| TCGA-AY-A69D-01 | Colorectal | C420R | E722K | | | | |
| | combined | | | | | | |
| TCGA-AZ-4315-01 | Colorectal | R88Q | H1048R | E81* | | | |
| | combined | | | | | | |
| TCGA-CA-5255-01 | Colorectal | E545A | H1047R | | | | |
| | combined | | | | | | |
| TCGA-CA-6718-01 | Colorectal | R88Q | H1047Q | M732I | | | |
| | combined | | | | | | |
| TCGA-DM-A0X9-01 | Colorectal | E545K | E970K | | | | |
| | combined | | | | | | |
| TCGA-DM-A28A-01 | Colorectal | H1047Y | V344G | | | | |
| | combined | | | | | | |
| TCGA-F4-6569-01 | Colorectal | H1047Y | R357Q | | | | |
| | combined | | | | | | |
| TCGA-F4-6807-01 | Colorectal | M1043I | R108H | | | | |
| | combined | | | | | | |
| TCGA-NH-A50T-01 | Colorectal | E542K | P104R | | | | |
| | combined | | | | | | |
| TCGA-QG-A5YX-01 | Colorectal | H1047R | E542G | | | | |
| | combined | | | | | | |
| 587220 | Colorectal | H1047R | K111N | | | | |
| | combined | | | | | | |
| 587224 | Colorectal | H1047R | K111E | | | | |
| | combined | | | | | | |
| 587260 | Colorectal | R88Q | E545A | | | | |
| | combined | | | | | | |
| 587356 | Colorectal | T1052K | H419N | | | | |
| | combined | | | | | | |
| P-0000788-T01-IM3 | Colorectal | E542K | M1010I | | | | |
| | combined | | | | | | |
| P-0001215-T01-IM3 | Colorectal | E365K | I816N | R992* | | | |
| | combined | | | | | | |
| P-0001289-T01-IM3 | Colorectal | H1047R | V344G | | | | |
| | combined | | | | | | |
| P-0001732-T01-IM3 | Colorectal | E542K | M1043I | | | | |
| | combined | | | | | | |
| P-0001940-T01-IM3 | Colorectal | H1047R | G118D | | | | |
| | combined | | | | | | |
| P-0002413-T01-IM3 | Colorectal | Q546K | E726K | | | | |
| | combined | | | | | | |
| P-0003513-T01-IM5 | Colorectal | E365K | H1047Q | | | | |
| | combined | | | | | | |
| P-0003720-T01-IM5 | Colorectal | H1047R | R93Q | | | | |
| | combined | | | | | | |
| P-0004566-T01-IM5 | Colorectal | E545K | H1047R | | | | |
| | combined | | | | | | |
| P-0004865-T01-IM5 | Colorectal | E542K | K111N | | | | |
| | combined | | | | | | |
| P-0004928-T01-IM5 | Colorectal | E545K | E542K | | | | |
| | combined | | | | | | |
| P-0006170-T01-IM5 | Colorectal | E545G | Q75H | | | | |
| | combined | | | | | | |
| P-0006581-T01-IM5 | Colorectal | E545K | L540F | | | | |
| | combined | | | | | | |
| P-0006612-T01-IM5 | Colorectal | M1043I | R693C | | | | |
| | combined | | | | | | |
| P-0007147-T01-IM5 | Colorectal | R88Q | H1047R | | | | |
| | combined | | | | | | |
| P-0007272-T01-IM5 | Colorectal | R38C | R357L | | | | |
| | combined | | | | | | |
| P-0007836-T01-IM5 | Colorectal | E110del | R93Q | E418D | | | |
| | combined | | | | | | |
| P-0008721-T01-IM5 | Colorectal | H1047R | A289T | | | | |
| | combined | | | | | | |
| P-0010125-T01-IM5 | Colorectal | G364R | V125E | | | | |
| | combined | | | | | | |
| P-0010167-T01-IM5 | Colorectal | H1047R | V851A | | | | |
| | combined | | | | | | |
| P-0011071-T01-IM5 | Colorectal | E542K | R108S | | | | |
| | combined | | | | | | |
| P-0011357-T01-IM5 | Colorectal | K111N | M1004I | R357* | | | |
| | combined | | | | | | |
| P-0013010-T01-IM5 | Colorectal | H1047R | C420R | | | | |
| | combined | | | | | | |
| P-0013020-T01-IM5 | Colorectal | H1047R | Q546R | | | | |
| | combined | | | | | | |
| coadread_dfci_2016_1240 | Colorectal | E542K | E726K | | | | |
| | combined | | | | | | |
| coadread_dfci_2016_1762 | Colorectal | Q546R | *1069Wext*4 | | | | |
| | combined | | | | | | |
| coadread_dfci_2016_2271 | Colorectal | E542K | F83S | | | | |
| | combined | | | | | | |
| coadread_dfci_2016_250 | Colorectal | G1007R | R93W | | | | |
| | combined | | | | | | |
| coadread_dfci_2016_2641 | Colorectal | H1047R | R38S | | | | |
| | combined | | | | | | |
| coadread_dfci_2016_2664 | Colorectal | E542K | E982K | | | | |
| | combined | | | | | | |
| coadread_dfci_2016_2936 | Colorectal | E545K | C604R | | | | |
| | combined | | | | | | |
| coadread_dfci_2016_3010 | Colorectal | K111E | V344E | | | | |
| | combined | | | | | | |
| coadread_dfci_2016_3022 | Colorectal | E545K | R88Q | | | | |
| | combined | | | | | | |
| coadread_dfci_2016_3237 | Colorectal | H1047R | R93W | | | | |
| | combined | | | | | | |
| coadread_dfci_2016_328 | Colorectal | G1007R | L1006H | | | | |
| | combined | | | | | | |
| coadread_dfci_2016_3298 | Colorectal | R88Q | C604R | | | | |
| | combined | | | | | | |
| coadread_dfci_2016_3535 | Colorectal | R88Q | H1047R | | | | |
| | combined | | | | | | |
| coadread_dfci_2016_3611 | Colorectal | R88Q | R818C | | | | |
| | combined | | | | | | |
| coadread_dfci_2016_4508 | Colorectal | E542K | G106V | | | | |
| | combined | | | | | | |
| coadread_dfci_2016_578 | Colorectal | R88Q | K111N | | | | |
| | combined | | | | | | |
| coadread_dfci_2016_60 | Colorectal | E542K | C378R | | | | |
| | combined | | | | | | |
| TCGA-AA-3821-01 | Colorectal | H1047L | M1043I | | | | |
| | combined | | | | | | |
| TCGA-AA-3837-01 | Colorectal | R88Q | N345K | | | | |
| | combined | | | | | | |
| TCGA-AA-3852-01 | Colorectal | E81K | G106R | E545Q | | | |
| | combined | | | | | | |
| TCGA-AA-A00N-01 | Colorectal | R357Q | Y1021C | V344A | | | |
| | combined | | | | | | |
| TCGA-CA-6717-01 | Colorectal | R88Q | E970K | S1015Y | | | |
| | combined | | | | | | |
| TCGA-CM-6162-01 | Colorectal | R88Q | R108H | | | | |
| | combined | | | | | | |
| P-0009189-T01-IM5 | Cutaneous | S67F | L402F | | | | |
| | Melanoma | | | | | | |
| P-0009752-T01-IM5 | Cutaneous | H14Y | S326F | | | | |
| | Melanoma | | | | | | |
| TCGA-DA-A1IB-06 | Cutaneous | E674D | Q682K | | | | |
| | Melanoma | | | | | | |
| P-0001198-T01-IM3 | Cutaneous | E542K | C24S | S66F | W780* | | |
| | Squamous Cell | | | | | | |
| | Carcinoma | | | | | | |
| TCGA-IC-A6RE-01 | Esophageal | E545K | K111N | | | | |
| | Adenocarcinoma | | | | | | |
| TCGA-XP-A8T7-01 | Esophageal | E726K | M441I | | | | |
| | Squamous Cell | | | | | | |
| | Carcinoma | | | | | | |
| PGM4 | Esophagogastric | E545K | I13S | | | | |
| | Adenocarcinoma | | | | | | |
| P-0004531-T01-IM5 | Glioma combined | E545G | C420R | | | | |
| TCGA-DU-A5TT-01 | Glioma combined | K111E | Q546E | | | | |
| TCGA-S9-A6TW-01 | Glioma combined | E110del | N345Y | | | | |
| P18_Rec | Glioma combined | E542K | L540V | | | | |
| P-0000500-T01-IM3 | Glioma combined | E545K | E542K | | | | |
| P-0002633-T01-IM3 | Glioma combined | Q546R | R88Q | | | | |
| P-0002695-T01-IM3 | Glioma combined | E545K | E116K | | | | |
| P-0008649-T01-IM5 | Glioma combined | H1047R | L436_P449dup | | | | |
| TCGA-06-0210-02 | Glioma combined | E545K | R38H | | | | |
| TCGA-06-0879-01 | Glioma combined | L455_G460delinsF | E453_G460delinsDDF | E453D | | | |
| TCGA-06-5416-01 | Glioma combined | R88Q | E81K | S292I | | | |
| TCGA-12-5301-01 | Glioma combined | R88Q | R108H | | | | |
| TCGA-19-5954-01 | Glioma combined | K111R | R4* | | | | |
| TCGA-HT-7481-01 | Glioma combined | E453del | G118D | M1043V | | | |
| OSCJM-PT01-166-T | Head and Neck | K111E | T1052A | | | | |
| | Carcinoma | | | | | | |
| TCGA-BA-A8YP-01 | Head and Neck | E418K | C420R | | | | |
| | Carcinoma | | | | | | |
| TCGA-CR-6471-01 | Head and Neck | R88Q | M1043V | | | | |
| | Carcinoma | | | | | | |
| TCGA-CR-7404-01 | Head and Neck | E545K | E542K | | | | |
| | Carcinoma | | | | | | |
| TCGA-CV-7568-01 | Head and Neck | P104L | Y606C | | | | |
| | Carcinoma | | | | | | |
| P-0001105-T01-IM3 | High-Grade | R108H | C378F | | | | |
| | Serous Ovarian | | | | | | |
| | Cancer | | | | | | |
| P-0002286-T01-IM3 | Intrahepatic | E545K | C420R | | | | |
| | Cholangio- | | | | | | |
| | carcinoma | | | | | | |
| LO3793 | Lung combined | R88Q | E418K | | | | |
| LUAD-S01473-Tumor | Lung combined | E545K | E453Q | | | | |
| P-0005985-T01-IM5 | Lung combined | PIK3CA- | E545K | | | | |
| | | intragenic | | | | | |
| P-0009431-T01-IM5 | Lung combined | E81K | R93W | | | | |
| TCGA-73-7499-01 | Lung combined | M123_P124delinsIA | M123I | P124A | MP123IA | | |
| P-0011388-T01-IMS | Lung combined | D1029H | D1045N | E982Q | | | |
| TCGA-18-5595-01 | Lung combined | E545K | E726K | | | | |
| TCGA-21-1078-01 | Lung combined | E542K | D538N | | | | |
| TCGA-33-A4WN-01 | Lung combined | E542K | P539S | F667L | | | |
| TCGA-60-2721-01 | Lung combined | E542K | G1049R | | | | |
| A14 | Lung combined | E529G | G1049S | | | | |
| MB-REC-31 | Medulloblastoma | H1047L | Q958K | | | | |
| PIP14-47205-T1 | Mixed Cancer | N345K | I391M | | | | |
| | Types | | | | | | |
| TCGA-VS-A9UT-01 | Mucinous | E418K | G106R | | | | |
| | Carcinoma | | | | | | |
| TCGA-VS-A9UZ-01 | Mucinous | E545K | E453Q | | | | |
| | Carcinoma | | | | | | |
| P-0002411-T01-IM3 | Nasopharyngeal | E545K | D538Y | | | | |
| | Carcinoma | | | | | | |
| P-0000650-T01-IM3 | Non- | K724del | E542K | | | | |
| | Seminomatous | | | | | | |
| | Germ Cell Tumor | | | | | | |
| P-0005212-T01-IM5 | Oropharynx | E542K | E78K | | | | |
| | Squamous Cell | | | | | | |
| | Carcinoma | | | | | | |
| P-0009761-T01-IM5 | Oropharynx | E545K | E579K | | | | |
| | Squamous Cell | | | | | | |
| | Carcinoma | | | | | | |
| TCGA-5P-A9K3-01 | Papillary Renal | E542K | R38C | | | | |
| | Cell Carcinoma | | | | | | |
| P-0003178-T01-IM5 | Poorly | E542K | H1047R | | | | |
| | Differentiated | | | | | | |
| | Thyroid Cancer | | | | | | |
| TCGA-EJ-A65D-01 | Prostate | *1069fs* | R88Q | | | | |
| | Adenocarcinoma | | | | | | |
| TCGA-HC-7081-01 | Prostate | E542A | N345I | | | | |
| | Adenocarcinoma | | | | | | |
| TCGA-XK-AAIW-01 | Prostate | R108C | L569I | | | | |
| | Adenocarcinoma | | | | | | |
| TCGA-AG-A015-01 | Rectal | Q546K | D350G | | | | |
| | Adenocarcinoma | | | | | | |
| TCGA-AH-6903-01 | Rectal | H1047R | E726K | | | | |
| | Adenocarcinoma | | | | | | |
| TCGA-EI-6917-01 | Rectal | R88Q | E116K | | | | |
| | Adenocarcinoma | | | | | | |
| TCGA-F5-6814-01 | Rectal | K337N | E469A | | | | |
| | Adenocarcinoma | | | | | | |
| P-0000957-T01-IM3 | Salivary Duct | *1069F | M1004I | | | | |
| | Carcinoma | | | | | | |
| TCGA-HU-8608-01 | Stomach | E365K | N345K | | | | |
| | combined | | | | | | |
| TCGA-VQ-A91K-01 | Stomach | G1049R | E453K | | | | |
| | combined | | | | | | |
| TCGA-VQ-A91W-01 | Stomach | E542K | G364R | | | | |
| | combined | | | | | | |
| TCGA-VQ-A8P2-01 | Stomach | R88Q | D350N | | | | |
| | combined | | | | | | |
| P-0009918-T01-IM5 | Stomach | H1047R | V344M | K111N | | | |
| | combined | | | | | | |
| TCGA-BR-4371-01 | Stomach | E545K | H1047R | | | | |
| | combined | | | | | | |
| TCGA-BR-6452-01 | Stomach | R412Q | H1047R | E547K | K179E | V243A | |
| | combined | | | | | | |
| TCGA-BR-6706-01 | Stomach | E545K | E453K | | | | |
| | combined | | | | | | |
| TCGA-BR-8284-01 | Stomach | E542K | Q546K | | | | |
| | combined | | | | | | |
| TCGA-BR-8366-01 | Stomach | G118D | Y182H | | | | |
| | combined | | | | | | |
| TCGA-BR-8591-01 | Stomach | R88Q | C378R | | | | |
| | combined | | | | | | |
| TCGA-BR-8676-01 | Stomach | E542K | E453K | | | | |
| | combined | | | | | | |
| TCGA-CG-5721-01 | Stomach | K111E | K948E | | | | |
| | combined | | | | | | |
| TCGA-F1-6177-01 | Stomach | R93Q | Q546H | | | | |
| | combined | | | | | | |
| TCGA-BR-8686-01 | Stomach | E542K | R88Q | | | | |
| | combined | | | | | | |
| TCGA-D7-5577-01 | Stomach | R88Q | M1043I | | | | |
| | combined | | | | | | |
| TCGA-HU-A4GQ-01 | Stomach | R93W | Y1021H | | | | |
| | combined | | | | | | |
| TCGA-VQ-A8PF-01 | Stomach | R88Q | G106V | | | | |
| | combined | | | | | | |
| DS-uttcc-042-P | Upper Tract | H1047L | D1029Y | | | | |
| | Urothelial | | | | | | |
| | Carcinoma | | | | | | |
| DS-uttcc-060-P | Upper Tract | F261V | Q958H | R349* | | | |
| | Urothelial | | | | | | |
| | Carcinoma | | | | | | |
| P-0004330-T01-IM5 | Upper Tract | R93Q | R310C | | | | |
| | Urothelial | | | | | | |
| | Carcinoma | | | | | | |
| P-0004688-T01-IM5 | Upper Tract | R108H | G1007D | T322I | | | |
| | Urothelial | | | | | | |
| | Carcinoma | | | | | | |
| P-0010803-T01-IM5 | Upper Tract | H1047Y | E542V | | | | |
| | Urothelial | | | | | | |
| | Carcinoma | | | | | | |
| MM04T | Uterine combined | R88Q | R357Q | | | | |
| MM18T | Uterine combined | R818C | R916C | R992* | | | |
| P-0002357-T01-IM3 | Uterine combined | H1047Y | D1029H | | | | |
| TCGA-N7-A4Y8-01 | Uterine combined | E545K | H1047R | | | | |
| TCGA-ND-A4WC-01 | Uterine combined | R88Q | R108H | | | | |
| P-0006269-T01-IM5 | Uterine combined | H1047Y | V344M | | | | |
| P-0000404-T01-IM3 | Uterine combined | K111E | P449R | | | | |
| P-0000448-T01-IM3 | Uterine combined | V344M | T1025A | | | | |
| P-0003767-T01-IM5 | Uterine combined | H1047R | E453del | | | | |
| P-0004136-T01-IM5 | Uterine combined | E542K | I1058L | | | | |
| P-0004255-T01-IM5 | Uterine combined | H1047R | R88Q | | | | |
| P-0006201-T01-IM5 | Uterine combined | R93W | E365K | | | | |
| P-0009316-T01-IM5 | Uterine combined | E545K | H1047Y | | | | |
| P-0010967-T01-IM5 | Uterine combined | R88Q | E81K | | | | |
| P-0011569-T01-IM5 | Uterine combined | R88Q | Y1021C | R93Q | L779M | | |
| P-0011570-T01-IM5 | Uterine combined | D549N | F83S | L339I | | | |
| P-0012113-T01-IM5 | Uterine combined | R88Q | T1025A | Q958R | | | |
| P-0012152-T01-IM5 | Uterine combined | E545G | P97H | | | | |
| P-0012358-T01-IM5 | Uterine combined | R88Q | L997I | R115Q | R537Q | | |
| P-0012397-T01-IM5 | Uterine combined | R88Q | Y1021C | R852Q | | | |
| TCGA-4E-A92E-01 | Uterine combined | E542K | M1043I | | | | |
| TCGA-A5-A0GB-01 | Uterine combined | R108H | G359R | | | | |
| TCGA-A5-A0GP-01 | Uterine combined | R88Q | G118D | | | | |
| TCGA-A5-A0RA-01 | Uterine combined | W11_P18del | R115L | | | | |
| TCGA-A5-A0VP-01 | Uterine combined | R88Q | E542V | | | | |
| TCGA-A5-A2K5-01 | Uterine combined | R88Q | M1043I | | | | |
| TCGA-AJ-A3BH-01 | Uterine combined | R108H | D1045A | | | | |
| TCGA-AJ-A3EK-01 | Uterine combined | R88Q | R93W | | | | |
| TCGA-AJ-A3EL-01 | Uterine combined | R88Q | I816S | | | | |
| TCGA-AJ-A8CW-01 | Uterine combined | R88Q | H1047R | | | | |
| TCGA-AP-A054-01 | Uterine combined | M1043I | L866W | | | | |
| TCGA-AP-A056-01 | Uterine combined | R88Q | Y1021C | | | | |
| TCGA-AP-A0LD-01 | Uterine combined | R38H | R108H | | | | |
| TCGA-AP-A0LM-01 | Uterine combined | R818C | D350N | N170S | L279I | D454Y | |
| TCGA-AP-A0LS-01 | Uterine combined | H1047Y | P449S | | | | |
| TCGA-AP-A1DK-01 | Uterine combined | R88Q | M811I | | | | |
| TCGA-AP-A1DV-01 | Uterine combined | E545G | T1025A | | | | |
| TCGA-AP-A1E0-01 | Uterine combined | R88Q | M1043I | F667L | | | |
| TCGA-AP-A1E1-01 | Uterine combined | Q546K | Y1021C | | | | |
| TCGA-AX-A05Z-01 | Uterine combined | T1025A | S576Y | L997I | | | |
| TCGA-AX-A060-01 | Uterine combined | C604R | V344A | | | | |
| TCGA-AX-A06J-01 | Uterine combined | C378F | G118D | | | | |
| TCGA-AX-A0J0-01 | Uterine combined | R38C | T1025S | | | | |
| TCGA-AX-A1C5-01 | Uterine combined | H1047Y | V344M | | | | |
| TCGA-AX-A1CE-01 | Uterine combined | R88Q | D350G | R818H | L929M | P953S | A1020T |
| TCGA-AX-A2HC-01 | Uterine combined | R88Q | R832* | X555_splice | | | |
| TCGA-AX-A2HD-01 | Uterine combined | E418K | R93Q | E600K | | | |
| TCGA-AX-A2HG-01 | Uterine combined | E542K | R310C | | | | |
| TCGA-AX-A2IN-01 | Uterine combined | N345T | R38C | | | | |
| TCGA-B5-A0JU-01 | Uterine combined | E545A | H1047Y | | | | |
| TCGA-B5-A0JY-01 | Uterine combined | G12D | P449L | E522A | | | |
| TCGA-B5-A0K2-01 | Uterine combined | R93W | C378R | | | | |
| TCGA-B5-A111-01 | Uterine combined | R93Q | M1004I | | | | |
| TCGA-B5-A11R-01 | Uterine combined | E39K | N1044K | | | | |
| TCGA-B5-A11S-01 | Uterine combined | M1043V | G1007R | | | | |
| TCGA-B5-A11X-01 | Uterine combined | G118D | E39K | | | | |
| TCGA-B5-A11Y-01 | Uterine combined | V344M | H1047Q | R93Q | | | |
| TCGA-B5-A3FA-01 | Uterine combined | R88Q | Y1021H | R401Q | | | |
| TCGA-B5-A3FC-01 | Uterine combined | Y1021C | R108C | | | | |
| TCGA-BG-A0M0-01 | Uterine combined | P471L | E365K | | | | |
| TCGA-BG-A0MQ-01 | Uterine combined | Y1021C | R93Q | | | | |
| TCGA-BG-A0VX-01 | Uterine combined | R88Q | Q546P | | | | |
| TCGA-BG-A0VZ-01 | Uterine combined | K111N | N1044K | | | | |
| TCGA-BG-A187-01 | Uterine combined | C420R | R93Q | | | | |
| TCGA-BK-A0C9-01 | Uterine combined | E453Q | E542Q | | | | |
| TCGA-BK-A56F-01 | Uterine combined | FNDC3B-PIK3CA | H1047R | P539R | | | |
| TCGA-BS-A0TC-01 | Uterine combined | R108H | R38C | | | | |
| TCGA-BS-A0UF-01 | Uterine combined | R401Q | Q643H | F1016C | | | |
| TCGA-BS-A0UJ-01 | Uterine combined | Q546P | W479* | | | | |
| TCGA-BS-A0UV-01 | Uterine combined | R88Q | R1023* | | | | |
| TCGA-BS-A0V6-01 | Uterine combined | K111E | D939G | | | | |
| TCGA-BS-A0V7-01 | Uterine combined | H1047L | N345K | | | | |
| TCGA-D1-A103-01 | Uterine combined | E39K | R617W | | | | |
| TCGA-D1-A16R-01 | Uterine combined | R88Q | C901F | | | | |
| TCGA-D1-A16Y-01 | Uterine combined | R88Q | E81K | | | | |
| TCGA-D1-A17F-01 | Uterine combined | G118D | D725N | | | | |
| TCGA-D1-A17T-01 | Uterine combined | R88Q | C901F | | | | |
| TCGA-D1-A1O5-01 | Uterine combined | Q546P | E542A | | | | |
| TCGA-D1-A1O7-01 | Uterine combined | R88Q | M1004V | | | | |
| TCGA-DF-A2KN-01 | Uterine combined | C378F | R617Q | R992* | | | |
| TCGA-DF-A2KU-01 | Uterine combined | R88Q | E365K | Y392H | | | |
| TCGA-DF-A2KV-01 | Uterine combined | T1025A | R93W | | | | |
| TCGA-E6-A1LX-01 | Uterine combined | R88Q | C378Y | L339I | P266T | F930V | |
| TCGA-EO-A22R-01 | Uterine combined | R88Q | G106V | R19I | | | |
| TCGA-EO-A22U-01 | Uterine combined | E365K | R93W | I351T | | | |
| TCGA-EO-A22X-01 | Uterine combined | R88Q | R357Q | M282V | L1006R | | |
| TCGA-EO-A3AV-01 | Uterine combined | E81K | M1004R | | | | |
| TCGA-EO-A3B0-01 | Uterine combined | D350N | R38C | R770Q | | | |
| TCGA-EO-A3KX-01 | Uterine combined | R108H | N1044I | | | | |
| TCGA-EY-A1GL-01 | Uterine combined | E726K | K111N | | | | |
| TCGA-EY-A1GU-01 | Uterine combined | R38H | G118D | | | | |
| TCGA-EY-A1H0-01 | Uterine combined | R38C | E542Q | M282V | | | |
| TCGA-EY-A2OM-01 | Uterine combined | R88Q | R108H | | | | |
| TCGA-FI-A2D5-01 | Uterine combined | R88Q | M1004I | T322A | | | |
| TCGA-PG-A916-01 | Uterine combined | G118D | N345I | | | | |
| P-0001821-T01-IM3 | Uterine combined | R88Q | Q546H | | | | |
| P-0004017-T01-IM5 | Uterine combined | H1047L | E453K | | | | |
| TCGA-DI-A1BU-01 | Uterine combined | E110del | G106D | Y165H | I406V | | |
| TCGA-E6-A2P8-01 | Uterine combined | R88Q | A1066V | | | | |
| TCGA-EO-A3AZ-01 | Uterine combined | E453del | R108H | E545Q | Q958R | | |
| TCGA-QF-A5YS-01 | Uterine combined | R88Q | T1025A | M1055I | | | |
| P-0002945-T01-IM3 | Uterine combined | H1047Y | E726K | | | | |
| TCGA-A5-A0G2-01 | Uterine combined | L569I | R852Q | G903E | | | |
| TCGA-A5-A0R6-01 | Uterine combined | M1043V | C378Y | | | | |
| TCGA-E6-A1LZ-01 | Uterine combined | E545K | R992P | E453Q | | | |

TABLE 5
List of multiple PIK3CA mutant tumors (n = 451) (MSK IMPACT)

| Sample ID | Cancer Type | PIK3CA Mutation |
|---|---|---|
| P-0006682-T02-IM6 | Anal Cancer | E545K, R108H |
| P-0018953-T01-IM6 | Anal Cancer | M1043I, K111N |
| P-0020781-T01-IM6 | Anal Cancer | E545K, RS3W |
| P-0031461-T01-IM6 | Anal Cancer | E545K, E726K |
| P-0000043-T02-IM3 | Bladder Cancer | E545K, V356A |
| P-0002659-T01-IM3 | Bladder Cancer | E545K, Q682H |
| P-0009101-T01-IM5 | Bladder Cancer | E545K, E542K |
| P-0000423-T01-IM3 | Bladder Cancer | E545K, E453Q |
| P-0003433-T01-IM5 | Bladder Cancer | E542K, E453K |
| P-0004330-T01-IM5 | Bladder Cancer | R93Q, R310C |
| P-0004424-T01-IM5 | Bladder Cancer | H1047R, E542K |
| P-0004688-T01-IM5 | Bladder Cancer | R108H, T322I, G1007D |
| P-0010803-T01-IM5 | Bladder Cancer | H1047Y, E542V |
| P-0010921-T01-IM5 | Bladder Cancer | E542K, H1065Y, E453Q |
| P-0014909-T01-IM6 | Bladder Cancer | E542K, E726K |
| P-0015886-T01-IM6 | Bladder Cancer | E726K, E503K |
| P-0017472-T01-IM6 | Bladder Cancer | Q75E, A1066V |
| P-0019005-T01-IM6 | Bladder Cancer | E545K, E542K |
| P-0024039-T01-IM6 | Bladder Cancer | E545K, E542K |
| P-0031416-T01-IM6 | Bladder Cancer | N345K, E978K |
| P-0031860-T01-IM6 | Bladder Cancer | E545K, D133Y |
| P-0032134-T01-IM6 | Bladder Cancer | E545K, R88Q, E453Q, E418K |
| P-0000138-T01-IM3 | Breast Cancer | E542K, E453K |
| P-0000155-T01-IM3 | Breast Cancer | H1047R, D350N |
| P-0000167-T01-IM3 | Breast Cancer | K111E, E418K |
| P-0000167-T02-IM3 | Breast Cancer | E542K, K111E |
| P-0000234-T01-IM3 | Breast Cancer | E542K, M1043I |
| P-0000397-T01-IM3 | Breast Cancer | E542K, E453K |
| P-0000607-T01-IM3 | Breast Cancer | H1047L, E545Q |
| P-0001114-T02-IM3 | Breast Cancer | E545K, E453Q, E978Q |
| P-0001351-T01-IM3 | Breast Cancer | E545K, E726K |
| P-0001902-T01-IM3 | Breast Cancer | E542K, E545D |
| P-0001990-T01-IM3 | Breast Cancer | N345K, H1047Y |
| P-0002124-T01-IM3 | Breast Cancer | H1047R, E365K |
| P-0002562-T01-IM3 | Breast Cancer | E545K, E453Q |
| P-0002667-T01-IM3 | Breast Cancer | H1047R, N107I |
| P-0002841-T01-IM3 | Breast Cancer | H1047R, K111N |
| P-0002922-T01-IM3 | Breast Cancer | H1047R, E545Q |
| P-0003224-T01-IM5 | Breast Cancer | H1047R, G106V |
| P-0003233-T03-IM6 | Breast Cancer | E542K, E453K |
| P-0003882-T03-IM6 | Breast Cancer | N345K, E726K |
| P-0003987-T01-IM5 | Breast Cancer | E545K, H1047R |
| P-0004187-T01-IM5 | Breast Cancer | H1047R, E970K |
| P-0004264-T01-IM5 | Breast Cancer | H1047R, E726K |
| P-0004433-T01-IM5 | Breast Cancer | E545K, L252F |
| P-0005032-T01-IM5 | Breast Cancer | L455Wfs*6, L452Qfs*5 |
| P-0005037-T01-IM5 | Breast Cancer | N345K, R93Q |
| P-0005154-T01-IM5 | Breast Cancer | H1047R, E542K |
| P-0005220-T01-IM5 | Breast Cancer | H1047R, C407F |
| P-0005242-T01-IM5 | Breast Cancer | E545K, E726K |
| P-0005818-T01-IM5 | Breast Cancer | H1047R, G118D |
| P-0006161-T03-IM5 | Breast Cancer | E542K, E453K |
| P-0006166-T01-IM5 | Breast Cancer | E542K, E726K |
| P-0006335-T01-IM5 | Breast Cancer | E545K, T1025A |
| P-0006660-T01-IM5 | Breast Cancer | R88Q, Q546H |
| P-0006723-T01-IM5 | Breast Cancer | H1047R, E81K |
| P-0006780-T01-IM5 | Breast Cancer | V344M, E726K |
| P-0006787-T01-IM5 | Breast Cancer | H1047R, E726K |
| P-0007396-T01-IM5 | Breast Cancer | N345K, E970K |
| P-0009364-T02-IM6 | Breast Cancer | E545K, H1047R |
| P-0010043-T01-IM5 | Breast Cancer | H1047R, P539R |
| P-0010703-T01-IM5 | Breast Cancer | H1047L, E726K |
| P-0010917-T01-IM5 | Breast Cancer | E542K, E726K |
| P-0011305-T01-IM5 | Breast Cancer | E545K, E726K |
| P-0011420-T01-IM5 | Breast Cancer | E545K, A1066V |
| P-0012667-T01-IM5 | Breast Cancer | D390N, E385K |
| P-0013491-T01-IM5 | Breast Cancer | G118D, E542Q |
| P-0013771-T01-IM5 | Breast Cancer | H1047R, E726K |
| P-0013895-T01-IM5 | Breast Cancer | E542K, E726K |
| P-0014091-T01-IM5 | Breast Cancer | E81K, H1047L |
| P-0014136-T01-IM5 | Breast Cancer | E542K, N457K |
| P-0014362-T01-IM6 | Breast Cancer | H1047R, N370K |
| P-0014656-T01-IM6 | Breast Cancer | E542K, K240Q |
| P-0015097-T01-IM6 | Breast Cancer | H1047R, E453K |
| P-0015379-T01-IM6 | Breast Cancer | H419_P421delinsQ, M1043_N1044delinsVK |
| P-0015499-T01-IM6 | Breast Cancer | H1047R, E418K |
| P-0015944-T01-IM6 | Breast Cancer | C420R, E726K |
| P-0015964-T01-IM6 | Breast Cancer | E545K, M1004I |
| P-0016473-T01-IM6 | Breast Cancer | E545K, L239R |
| P-0016686-T01-IM6 | Breast Cancer | H1047R, P539R |
| P-0017015-T01-IM6 | Breast Cancer | N345K, E726K |
| P-0017042-T01-IM6 | Breast Cancer | E545K, M1043I |
| P-0017422-T01-IM6 | Breast Cancer | E542K, A1066L |
| P-0018661-T01-IM6 | Breast Cancer | E453K, G451K |
| P-0019024-T01-IM6 | Breast Cancer | E545K, L10_P18del |
| P-0019040-T01-IM6 | Breast Cancer | H1047R, R93Q, E418K |
| P-0019103-T01-IM6 | Breast Cancer | H1047R, L456R |
| P-0019118-T01-IM6 | Breast Cancer | E542K, E453K |
| P-0019425-T01-IM6 | Breast Cancer | H1047R, C257Y |
| P-0019458-T01-IM6 | Breast Cancer | H1047R, E726K |
| P-0020188-T01-IM6 | Breast Cancer | E542K, E726K |
| P-0020301-T01-IM6 | Breast Cancer | E453K, H419_P421delinsQ |
| P-0021148-T01-IM6 | Breast Cancer | H1047R, V344M |
| P-0021480-T01-IM6 | Breast Cancer | H1047R, E81K |
| P-0021571-T01-IM6 | Breast Cancer | H1047R, R88Q |
| P-0021751-T01-IM6 | Breast Cancer | Q546R, E453K |
| P-0021895-T01-IM6 | Breast Cancer | H1047R, E726K |
| P-0022762-T01-IM6 | Breast Cancer | H1047R, G451V |
| P-0023508-T01-IM6 | Breast Cancer | E545K, K228N |
| P-0023835-T01-IM6 | Breast Cancer | H1047R, E722K |
| P-0024230-T01-IM6 | Breast Cancer | K111E, E545V |
| P-0024231-T01-IM6 | Breast Cancer | Q546R, E453_L455del |
| P-0024285-T01-IM6 | Breast Cancer | E545K, E726K |
| P-0024507-T01-IM6 | Breast Cancer | E545K, H1047R |
| P-0025307-T01-IM6 | Breast Cancer | E110del, E453K |
| P-0025489-T01-IM6 | Breast Cancer | E542K, E726K |
| P-0025528-T01-IM6 | Breast Cancer | H1047R, Q546H |
| P-0026513-T01-IM6 | Breast Cancer | E542K, G118D |
| P-0026885-T01-IM6 | Breast Cancer | H1047R, M1004I, E722K |
| P-0027710-T01-IM6 | Breast Cancer | E110del, P539R |
| P-0028309-T01-IM6 | Breast Cancer | H1047R, E39Q |
| P-0028907-T01-IM6 | Breast Cancer | N345K, E726K |
| P-0028960-T01-IM6 | Breast Cancer | G118D, E982D |
| P-0029802-T01-IM6 | Breast Cancer | E545K, E542K |
| P-0030451-T01-IM6 | Breast Cancer | H1047R, R88Q |
| P-0031280-T01-IM6 | Breast Cancer | E545K, V344M |
| P-0031695-T01-IM6 | Breast Cancer | E542K, W1043I |
| P-0031839-T01-IM6 | Breast Cancer | E545K, M1004I |
| P-0000107-T01-IM3 | Breast Cancer | Y1021H, D1017E, I1019V |
| P-0000207-T01-IM3 | Breast Cancer | N345K, K111E |
| P-0000356-T01-IM3 | Breast Cancer | H1047R, E453K |
| P-0000381-T01-IM3 | Breast Cancer | H1047R, E726K |
| P-0000592-T01-IM3 | Breast Cancer | E545K, M1043I |
| P-0001043-T01-IM3 | Breast Cancer | H1047R, R93L |
| P-0001631-T01-IM3 | Breast Cancer | E545K, M1043L |
| P-0002657-T01-IM3 | Breast Cancer | G118D, G364R |
| P-0002756-T02-IM5 | Breast Cancer | E545K, E453Q |
| P-0004194-T01-IM5 | Breast Cancer | E545K, D725G |
| P-0004196-T01-IM5 | Breast Cancer | H1047R, Q958K |
| P-0004913-T01-IM5 | Breast Cancer | H1047R, E542A |
| P-0005120-T01-IM5 | Breast Cancer | E542K, E726K |
| P-0005314-T01-IM5 | Breast Cancer | E542K, E726K |
| P-0005611-T01-IM5 | Breast Cancer | E39K, M1043I |
| P-0005968-T01-IM5 | Breast Cancer | H1047R, R88Q |
| P-0008679-T01-IM5 | Breast Cancer | H1047R, N345I |
| P-0008694-T01-IM5 | Breast Cancer | E542K, E453K |
| P-0008845-T01-IM5 | Breast Cancer | G118D, K111E |
| P-0009745-T02-IM6 | Breast Cancer | C420R, E726K |
| P-0010002-T01-IM5 | Breast Cancer | G1049R, Q546P |
| P-0011355-T01-IM5 | Breast Cancer | H1047R, E81K |
| P-0012635-T02-IM6 | Breast Cancer | H1047R, P539R |
| P-0012911-T01-IM5 | Breast Cancer | C420R, R108H |
| P-0014278-T01-IM6 | Breast Cancer | E542K, E726K |
| P-0014479-T01-IM6 | Breast Cancer | E545K, E542Q |
| P-0014480-T01-IM6 | Breast Cancer | E542K, E726K |
| P-0014515-T01-IM6 | Breast Cancer | Q546R, M1004I |
| P-0014622-T01-IM6 | Breast Cancer | H1047R, P104L |
| P-0014737-T01-IM6 | Breast Cancer | H1047L, P539R |
| P-0014860-T01-IM6 | Breast Cancer | H1047L, E545D |
| P-0014940-T01-IM6 | Breast Cancer | E542K, E726K |
| P-0014943-T01-IM6 | Breast Cancer | H1047R, E542G |
| P-0015005-T01-IM6 | Breast Cancer | E545K, Q546R |
| P-0015633-T01-IM6 | Breast Cancer | H1047L, I112F |
| P-0015640-T01-IM6 | Breast Cancer | N1044K, D350N |
| P-0016041-T02-IM6 | Breast Cancer | R88Q, H1047L |
| P-0016773-T01-IM6 | Breast Cancer | G106V, Q546H |
| P-0016786-T01-IM6 | Breast Cancer | E545K, M10431 |
| P-0016802-T01-IM6 | Breast Cancer | H1047R, G118D |
| P-0016840-T01-IM6 | Breast Cancer | H1047R, V344M |
| P-0017581-T01-IM5 | Breast Cancer | E545G, T1025A |
| P-0017818-T01-IM6 | Breast Cancer | E545K, H1048R |
| P-0018891-T01-IM6 | Breast Cancer | H1047R, N107Y |
| P-0019133-T01-IM6 | Breast Cancer | G1049R, P539R |
| P-0019191-T01-IM6 | Breast Cancer | H1047R, E542K |
| P-0019808-T01-IM6 | Breast Cancer | H1047R, R108H |
| P-0019935-T01-IM6 | Breast Cancer | H1047R, E970K |
| P-0020020-T01-IM6 | Breast Cancer | D549N, *1fs* |
| P-0020043-T01-IM6 | Breast Cancer | D725N, H450_P458del |
| P-0020049-T01-IM6 | Breast Cancer | E545K, H1047R |
| P-0020551-T02-IM6 | Breast Cancer | E545K, E726K |
| P-0020645-T01-IM6 | Breast Cancer | H1047R, M1043V |
| P-0021040-T01-IM6 | Breast Cancer | H1047R, M1055L |
| P-0021087-T01-IM6 | Breast Cancer | E542K, T544S |
| P-0021129-T01-IM6 | Breast Cancer | H1047R, E726K |
| P-0021135-T02-IM6 | Breast Cancer | H1047R, E542Q |
| P-0021201-T01-IM6 | Breast Cancer | N345I, M1040T, L1036S |
| P-0021218-T01-IM6 | Breast Cancer | H1047R, E418K |
| P-0021660-T01-IM6 | Breast Cancer | E545K, E542K |
| P-0022021-T01-IM6 | Breast Cancer | E545K, N1044K, D549H |
| P-0022048-T01-IM6 | Breast Cancer | N345K, M1043_N1044delinsIH |
| P-0022088-T01-IM6 | Breast Cancer | E545K, H1047R |
| P-0022158-T01-IM6 | Breast Cancer | P471L, H1047C |
| P-0022167-T01-IM6 | Breast Cancer | G1049R, R93Q, P539R |
| P-0022565-T01-IM6 | Breast Cancer | H1047R, C378F |
| P-0022750-T01-IM6 | Breast Cancer | H1047R, E542K |
| P-0023131-T01-IM6 | Breast Cancer | H1047R, C420R |
| P-0023919-T02-IM6 | Breast Cancer | H1047R, C378Y |
| P-0024141-T01-IM6 | Breast Cancer | E542K, E726K |
| P-0024364-T01-IM6 | Breast Cancer | H1047R, P449A |
| P-0024420-T01-IM6 | Breast Cancer | E545K, D939G, D549N |
| P-0024647-T01-IM6 | Breast Cancer | H1047R, R88Q |
| P-0025190-T01-IM6 | Breast Cancer | E542K, E726K |
| P-0026012-T01-IM6 | Breast Cancer | E545K, H1047L |
| P-0026070-T01-IM6 | Breast Cancer | E545K, G903A |
| P-0026484-T01-IM6 | Breast Cancer | I31M, I112T |
| P-0026829-T01-IM6 | Breast Cancer | E542K, R93W |
| P-0026957-T01-IM6 | Breast Cancer | H1047R, D350G |
| P-0028922-T01-IM6 | Breast Cancer | G1049R, P104T |
| P-0029706-T01-IM6 | Breast Cancer | H1047R, C378Y |
| P-0029966-T01-IM6 | Breast Cancer | E545K, N1044K |
| P-0030165-T01-IM6 | Breast Cancer | N345K, E418K |
| P-0030169-T01-IM6 | Breast Cancer | R108H, H1047Q |
| P-0030392-T01-IM6 | Breast Cancer | E545K, E453K |
| P-0030977-T01-IM6 | Breast Cancer | H1047R, R108C |
| P-0031023-T01-IM6 | Breast Cancer | H1047R, R88Q |
| P-0031133-T01-IM6 | Breast Cancer | H1047R, S323F |
| P-0032388-T01-IM6 | Breast Cancer | E542K, E418K |
| P-0004374-T01-IM5 | Cancer of Unknown Primary | E542K, D454G |
| P-0008153-T01-IM5 | Cancer of Unknown Primary | H1047R, R88Q |
| P-0022966-T01-IM6 | Cancer of Unknown Primary | M1043I, I932M |
| P-0023748-T01-IM6 | Cancer of Unknown Primary | E545K, H1047R |
| P-0026350-T01-IM6 | Cancer of Unknown Primary | R108_E109del, X353_splice |
| P-0028427-T01-IM6 | Cancer of Unknown Primary | R357Q, K111N, R537Q, L62I, V146A |
| P-0010527-T01-IM5 | Cervical Cancer | E545K, E542K |
| P-0013774-T01-IM5 | Cervical Cancer | E545K, E81K |
| P-0015136-T01-IM6 | Cervical Cancer | C420R, N370K |
| P-0029938-T01-IM6 | Cervical Cancer | E545K, E453K |
| P-0030876-T01-IM6 | Cervical Cancer | E545K, E726K |
| P-0033211-T01-IM6 | Cervical Cancer | K111N, Q546L |
| P-0001215-T01-IM3 | Colorectal Cancer | E365K, R992*, I816N |
| P-0001732-T01-IM3 | Colorectal Cancer | E542K, M1043I |
| P-0011071-T01-IM5 | Colorectal Cancer | E542K, R108S |
| P-0019593-T01-IM6 | Colorectal Cancer | E542K, E453K |
| P-0020473-T01-IM6 | Colorectal Cancer | R88Q, I816S |
| P-0020493-T01-IM6 | Colorectal Cancer | H1047R, R93W |
| P-0022090-T01-IM6 | Colorectal Cancer | H1047R, Y343F |
| P-0023461-T01-IM6 | Colorectal Cancer | E545K, G106V |
| P-0026678-T01-IM6 | Colorectal Cancer | R38C, Y1021C |
| P-0000788-T01-IM3 | Colorectal Cancer | E542K, M1010I |
| P-0001289-T01-IM3 | Colorectal Cancer | H1047R, V344G |
| P-0001940-T01-IM3 | Colorectal Cancer | H1047R, G118D |
| P-0002413-T01-IM3 | Colorectal Cancer | Q546K, E726K |
| P-0003513-T01-IM5 | Colorectal Cancer | E365K, H1047Q |
| P-0003720-T01-IM5 | Colorectal Cancer | H1047R, R93Q |
| P-0004566-T01-IM5 | Colorectal Cancer | E545K, H1047R |
| P-0004865-T01-IM5 | Colorectal Cancer | E542K, K111N |
| P-0004928-T01-IM5 | Colorectal Cancer | E545K, E542K |
| P-0005270-T02-IM6 | Colorectal Cancer | H1065Y, E39D |
| P-0006170-T01-IM5 | Colorectal Cancer | E545G, Q75H |
| P-0006581-T01-IM5 | Colorectal Cancer | E545K, L540F |
| P-0006612-T01-IM5 | Colorectal Cancer | M1043I, R693C |
| P-0007147-T01-IM5 | Colorectal Cancer | H1047R, R88Q |
| P-0007272-T01-IM5 | Colorectal Cancer | R38C, R357L |
| P-0007836-T01-IM5 | Colorectal Cancer | E110del, R93Q, E418D |
| P-0008721-T01-IM5 | Colorectal Cancer | H1047R, A289T |
| P-0010125-T01-IM5 | Colorectal Cancer | G364R, V125E |
| P-0010167-T01-IM5 | Colorectal Cancer | H1047R, V851A |
| P-0011357-T01-IM5 | Colorectal Cancer | M1004I, K111N, R357* |
| P-0013010-T01-IM5 | Colorectal Cancer | H1047R, C420R |
| P-0013020-T01-IM5 | Colorectal Cancer | H1047R, Q546R |
| P-0016066-T01-IM6 | Colorectal Cancer | H1047R, K111E |
| P-0016314-T01-IM6 | Colorectal Cancer | H1047R, C420R |
| P-0017583-T01-IM5 | Colorectal Cancer | Y1021C, H1047Q |
| P-0017995-T01-IM6 | Colorectal Cancer | Y1021H, R93W |
| P-0018437-T01-IM6 | Colorectal Cancer | C420R, H1047Y |
| P-0019464-T01-IM6 | Colorectal Cancer | N345K, H1047Y |
| P-0020144-T01-IM6 | Colorectal Cancer | R38C, Y1021H |
| P-0020383-T01-IM6 | Colorectal Cancer | R108H, N345I |
| P-0020689-T01-IM6 | Colorectal Cancer | R88Q, R108H |
| P-0022477-T01-IM6 | Colorectal Cancer | H1047R, K111N |
| P-0023187-T01-IM6 | Colorectal Cancer | D350N, K111N |
| P-0023271-T01-IM6 | Colorectal Cancer | R88Q, L339F, L144M |
| P-0025047-T01-IM6 | Colorectal Cancer | H1047R, K111E |
| P-0025073-T01-IM6 | Colorectal Cancer | E545K, H1047R |
| P-0025348-T01-IM6 | Colorectal Cancer | H1047R, P449S |
| P-0025695-T01-IM6 | Colorectal Cancer | E545K, E545G |
| P-0025715-T01-IM6 | Colorectal Cancer | H1047R, G914R |
| P-0025792-T01-IM6 | Colorectal Cancer | E542K, L540_S541dup |
| P-0026962-T01-IM6 | Colorectal Cancer | E542K, H1048R, V491Dfs*2 |
| P-0027476-T01-IM6 | Colorectal Cancer | H1047R, R88Q, P539R |
| P-0027608-T01-IM6 | Colorectal Cancer | E542K, R93Q |
| P-0030265-T01-IM6 | Colorectal Cancer | R93Q, T1025I |
| P-0030988-T01-IM6 | Colorectal Cancer | H1047R, R412* |
| P-0032698-T01-IM6 | Colorectal Cancer | H1047R, R1023Q |
| P-0033100-T01-IM6 | Colorectal Cancer | H1047R, R88Q, P266H |
| P-0033196-T01-IM6 | Colorectal Cancer | E545K, C420R |
| P-0000404-T01-IM3 | Endometrial Cancer | K111E, P449R |
| P-0000448-T01-IM3 | Endometrial Cancer | V344M, T1025A |
| P-0003767-T01-IM5 | Endometrial Cancer | H1047R, E453del |
| P-0004136-T01-IM5 | Endometrial Cancer | E542K, I1058L |
| P-0006269-T01-IM5 | Endometrial Cancer | V344M, H1047Y |
| P-0012152-T01-IM5 | Endometrial Cancer | E545G, P97H |
| P-0012358-T01-IM5 | Endometrial Cancer | R88Q, R115Q, R537Q, L997I |
| P-0014720-T01-IM6 | Endometrial Cancer | G118D, E110del |
| P-0018542-T01-IM6 | Endometrial Cancer | G118D, T957I |
| P-0019471-T01-IM6 | Endometrial Cancer | P104L, M1043I |
| P-0024756-T01-IM6 | Endometrial Cancer | H1047R, R108H |
| P-0025003-T01-IM6 | Endometrial Cancer | R88Q, E453G |
| P-0025812-T01-IM6 | Endometrial Cancer | M1043V, P539R |
| P-0026297-T01-IM6 | Endometrial Cancer | R88Q, D350G, R230* |
| P-0027698-T01-IM6 | Endometrial Cancer | H1047R, Y1021H |
| P-0028924-T01-IM6 | Endometrial Cancer | V344M, M1043I |
| P-0029337-T01-IM6 | Endometrial Cancer | V346E, D743V |
| P-0029683-T01-IM6 | Endometrial Cancer | H1047Q, F477S |
| P-0031387-T01-IM6 | Endometrial Cancer | V344M, N1044S, E418K |
| P-0031615-T01-IM6 | Endometrial Cancer | H1047R, M1043I |
| P-0002357-T01-IM3 | Endometrial Cancer | H1047Y, D1029H |
| P-0002945-T01-IM3 | Endometrial Cancer | H1047Y, E726K |
| P-0004017-T01-IM5 | Endometrial Cancer | H1047L, E453K |
| P-0004255-T01-IM5 | Endometrial Cancer | H1047R, R88Q |
| P-0006201-T01-IM5 | Endometrial Cancer | E365K, R93W |
| P-0009316-T01-IM5 | Endometrial Cancer | E545K, H1047Y |
| P-0010967-T01-IM5 | Endometrial Cancer | E81K, R88Q |
| P-0011569-T01-IM5 | Endometrial Cancer | R88Q, Y1021C, R93Q, L779M |
| P-0011570-T01-IM5 | Endometrial Cancer | L339I, D549N, F83S |
| P-0012113-T01-IM5 | Endometrial Cancer | R88Q, T1025A, Q958R |
| P-0012397-T01-IM5 | Endometrial Cancer | R88Q, Y1021C, R852Q |
| P-0012445-T01-IM5 | Endometrial Cancer | Y1021C, R992* |
| P-0012670-T01-IM5 | Endometrial Cancer | R88Q, Y1021C |
| P-0012726-T01-IM5 | Endometrial Cancer | R88Q, G106V |
| P-0012755-T01-IM5 | Endometrial Cancer | C420R, R108H |
| P-0012819-T01-IM5 | Endometrial Cancer | R38C, L712I |
| P-0012881-T01-IM5 | Endometrial Cancer | I816S, R19I |
| P-0013452-T01-IM5 | Endometrial Cancer | R93W, C378R |
| P-0013512-T01-IM5 | Endometrial Cancer | R88Q, G118D, G106D |
| P-0014388-T01-IM6 | Endometrial Cancer | R88Q, R93W |
| P-0014461-T01-IM6 | Endometrial Cancer | W11R, L1036S |
| P-0014495-T01-IM6 | Endometrial Cancer | R88Q, Q546R |
| P-0014528-T01-IM6 | Endometrial Cancer | R88Q, Y1021C |
| P-0014780-T01-IM6 | Endometrial Cancer | H1047R, R88Q, V344A, R852Q |
| P-0015626-T01-IM6 | Endometrial Cancer | R88Q, R357Q |
| P-0015869-T01-IM6 | Endometrial Cancer | R357Q, K111N, D883G |
| P-0015885-T01-IM6 | Endometrial Cancer | C420R, Y1021C |
| P-0015986-T01-IM6 | Endometrial Cancer | G118D, H1048R |
| P-0016099-T01-IM6 | Endometrial Cancer | E365K, F1002V |
| P-0016203-T01-IM6 | Endometrial Cancer | V344M, R88Q |
| P-0016247-T01-IM6 | Endometrial Cancer | H1047R, N345D |
| P-0016537-T01-IM6 | Endometrial Cancer | E545K, H1047L |
| P-0016556-T01-IM6 | Endometrial Cancer | C901F, R88Q |
| P-0016904-T01-IM6 | Endometrial Cancer | R88Q, N345K, R93W |
| P-0016907-T01-IM6 | Endometrial Cancer | P366R, H1047Y |
| P-0017302-T01-IM6 | Endometrial Cancer | Y1021H, K111E |
| P-0017424-T01-IM6 | Endometrial Cancer | E545K, R88Q |
| P-0017681-T01-IM6 | Endometrial Cancer | R88Q, R108H, V344A |
| P-0017896-T01-IM6 | Endometrial Cancer | E545K, E542K |
| P-0018005-T01-IM6 | Endometrial Cancer | E545K, R274T, L285F |
| P-0018009-T01-IM6 | Endometrial Cancer | E81K, E453K |
| P-0018459-T01-IM6 | Endometrial Cancer | H1047L, V101del |
| P-0018616-T01-IM6 | Endometrial Cancer | R108C, D743N |
| P-0018778-T01-IM6 | Endometrial Cancer | R115P, P539R |
| P-0018781-T01-IM6 | Endometrial Cancer | R88Q, P449S |
| P-0018790-T01-IM6 | Endometrial Cancer | H1047R, R108H |
| P-0019107-T01-IM6 | Endometrial Cancer | K111N, D350N |
| P-0019658-T01-IM6 | Endometrial Cancer | R88Q, R357Q |
| P-0019659-T01-IM6 | Endometrial Cancer | R88Q, R108S |
| P-0019741-T01-IM6 | Endometrial Cancer | R88Q, H1047Y |
| P-0019871-T01-IM6 | Endometrial Cancer | R88Q, F83I, P458L |
| P-0019986-T01-IM6 | Endometrial Cancer | R88Q, R108C |
| P-0020227-T01-IM6 | Endometrial Cancer | R38H, R38C, E600K |
| P-0020295-T01-IM6 | Endometrial Cancer | E81K, T1025A |
| P-0020509-T01-IM6 | Endometrial Cancer | R38H, R108H |
| P-0020556-T01-IM6 | Endometrial Cancer | E545K, R93Q |
| P-0020757-T01-IM6 | Endometrial Cancer | K111E, P539R |
| P-0021579-T01-IM6 | Endometrial Cancer | N1044K, N345H |
| P-0021665-T01-IM6 | Endometrial Cancer | R38H, E453K |
| P-0021777-T01-IM6 | Endometrial Cancer | R38C, R108H |
| P-0022813-T01-IM6 | Endometrial Cancer | K111E, H450_I459delinsL |
| P-0023250-T01-IM6 | Endometrial Cancer | H1047R, F83C |
| P-0023555-T01-IM6 | Endometrial Cancer | H1047R, D350G, E453K |
| P-0023857-T01-IM6 | Endometrial Cancer | H1047R, R88Q |
| P-0023969-T01-IM6 | Endometrial Cancer | R88Q, K111E |
| P-0025632-T01-IM6 | Endometrial Cancer | R88Q, R108H |
| P-0025648-T01-IM6 | Endometrial Cancer | R88Q, R357Q |
| P-0025662-T01-IM6 | Endometrial Cancer | N345T, E542G |
| P-0026278-T01-IM6 | Endometrial Cancer | R88Q, Y1021C, G364R |
| P-0026297-T02-IM6 | Endometrial Cancer | R88Q, D350G, R401Q, R230* |
| P-0026714-T01-IM6 | Endometrial Cancer | G12D, L422W |
| P-0027431-T01-IM6 | Endometrial Cancer | C420R, M1040K |
| P-0027652-T01-IM6 | Endometrial Cancer | E542A, F744L |
| P-0028610-T01-IM6 | Endometrial Cancer | E726K, P104L |
| P-0028776-T01-IM6 | Endometrial Cancer | H1047R, R951C, L267M, E784D |
| P-0028970-T01-IM6 | Endometrial Cancer | R38H, C378R |
| P-0029162-T01-IM6 | Endometrial Cancer | C378R, R4Q |
| P-0029274-T01-IM6 | Endometrial Cancer | E542K, N107I |
| P-9029666-T01-IM6 | Endometrial Cancer | P366R, E726A |
| P-0029690-T01-IM6 | Endometrial Cancer | R38C, T1025A, L339I, K111N |
| P-0029778-T01-IM6 | Endometrial Cancer | E81K, M1004I |
| P-0030372-T01-IM6 | Endometrial Cancer | R357Q, R992*, G1009E |
| P-0031042-T01-IM6 | Endometrial Cancer | E110del, R93Q |
| P-0031185-T02-IM6 | Endometrial Cancer | E542K, R88Q |
| P-0031195-T01-IM6 | Endometrial Cancer | Q546R, D350G |
| P-0031271-T01-IM6 | Endometrial Cancer | H1047R, V344M |
| P-0031332-T02-IM6 | Endometrial Cancer | H1047Y, R93W |
| P-0031501-T01-IM6 | Endometrial Cancer | V344M, R88Q, H1047Y |
| P-0032113-T01-IM6 | Endometrial Cancer | R88Q, Q981H |
| P-0032496-T01-IM6 | Endometrial Cancer | R88Q, R108H, R357Q |
| P-0032589-T01-IM6 | Endometrial Cancer | R88Q, Y1021C, E365K, T544N |
| P-0032606-T01-IM6 | Endometrial Cancer | C420R, I1058L |
| P-0032818-T01-IM6 | Endometrial Cancer | R88Q, P449S, R115Q |
| P-0033016-T01-IM6 | Endometrial Cancer | T1025A, K111N |
| P-0024054-T01-IM6 | Esophagogastric Cancer | E545K, M1004I |
| P-0009918-T01-IM5 | Esophagogastric Cancer | H1047R, V344M, K111N |
| P-0031190-T01-IM6 | Esophagogastric Cancer | X765_splice, X806_splice, X889_splice, X928_splice |
| P-0000650-T01-IM3 | Germ Cell Tumor | E542K, K724del |
| P-0019519-T01-IM6 | Glioma | E542A, E722K |
| P-0000500-T01-IM3 | Glioma | E545K, E542K |
| P-0002633-T01-IM3 | Glioma | R88Q, Q546R |
| P-0002695-T01-IM3 | Glioma | E545K, E116K |
| P-0004531-T01-IM5 | Glioma | C420R, E545G |
| P-0008649-T01-IM5 | Glioma | H1047R, L436_P449dup |
| P-0011521-T01-IM5 | Glioma | E542K, G106V |
| P-0013293-T02-IM6 | Glioma | E545K, D300N |
| P-0017482-T01-IM6 | Glioma | V71I, V448dup |
| P-0017675-T01-IM5 | Glioma | V125L, D133N, X382_splice |
| P-0018691-T01-IM6 | Glioma | G914R, N345D |
| P-0023123-T01-IM6 | Glioma | R93W, P2L, G411S |
| P-0023566-T01-IM6 | Glioma | E545K, R93W |
| P-0027147-T01-IM6 | Glioma | K111E, P539R |
| P-0029104-T01-IM6 | Glioma | P200S, P57L |
| P-0031276-T01-IM6 | Glioma | E542K, I543F |
| P-0032337-T01-IM6 | Glioma | Q546K, M1043V |
| P-0005212-T01-IM5 | Head and Neck Cancer | E542K, E78K |
| P-0009761-T01-IM5 | Head and Neck Cancer | E545K, E579K |
| P-0029015-T01-IM6 | Head and Neck Cancer | E545K, E542K |
| P-0002411-T01-IM3 | Head and Neck Cancer | E545K, D538Y |
| P-0024866-T01-IM6 | Head and Neck Cancer | E545K, E542K, D549N |
| P-0032593-T01-IM6 | Head and Neck Cancer | N1044K, E453del |
| P-0002286-T01-IM3 | Hepatobiliary Cancer | E545K, C420R |
| P-0023198-T01-IM6 | Hepatobiliary Cancer | E545K, E81K |
| P-0028463-T01-IM6 | Hepatobiliary Cancer | E545K, D549V |
| P-0009189-T01-IM5 | Melanoma | S67F, L402F |
| P-0009752-T01-IM5 | Melanoma | H14Y, S326F |
| P-0021394-T01-IM6 | Melanoma | E545K, C420R |
| P-0009431-T01-IM5 | Non-Small Cell Lung Cancer | E81K, R93W |
| P-0021735-T01-IM6 | Non-Small Cell Lung Cancer | H1047R, G118V |
| P-0022891-T03-IM6 | Non-Small Cell Lung Cancer | H1047R, E726K |
| P-0023265-T01-IM6 | Non-Small Cell Lung Cancer | E545K, E542K |
| P-0032172-T01-IM6 | Non-Small Cell Lung Cancer | E542K, M318V |
| P-0003551-T02-IM6 | Non-Small Cell Lung Cancer | E545K, E542K |
| P-0011388-T01-IM5 | Non-Small Cell Lung Cancer | D1029H, D1045N, E982Q |
| P-0012580-T01-IM5 | Non-Small Cell Lung Cancer | E726K, H1065L |
| P-0018244-T01-IM6 | Non-Small Cell Lung Cancer | E542K, P471A |
| P-0024921-T01-IM6 | Non-Small Cell Lung Cancer | P217A, N444I |
| P-0027048-T01-IM6 | Non-Small Cell Lung Cancer | H450_I459del, G460S |
| P-0028051-T01-IM6 | Non-Small Cell Lung Cancer | A77V, A415Cfs*2 |
| P-0032292-T01-IM6 | Non-Small Cell Lung Cancer | E542K, E453K |
| P-0032614-T01-IM6 | Non-Small Cell Lung Cancer | E545K, E542K |
| P-0033200-T01-IM6 | Non-Small Cell Lung Cancer | E542K, D214Y |
| P-0029643-T01-IM6 | Ovarian Cancer | V344M, R115L |
| P-0001105-T01-IM3 | Ovarian Cancer | R108H, C378F |
| P-0031332-T01-IM6 | Ovarian Cancer | H1047Y, E545D |
| P-0026409-T01-IM6 | Prostate Cancer | E542K, I1058L |
| P-0025880-T01-IM6 | Prostate Cancer | E545K, H1047L |
| P-0000957-T01-IM3 | Salivary Gland Cancer | M1004I, *1069Ffs*5 |
| P-0001198-T01-IM3 | Skin Cancer, Non-Melanoma | E542K, C24S, S66F, W780* |
| P-0018328-T01-IM6 | Skin Cancer, Non-Melanoma | H1047R, E726K |
| P-0018382-T01-IM6 | Small Bowel Cancer | R88Q, Q546R |
| P-0032985-T01-IM6 | Small Bowel Cancer | K111E, N107H |
| P-0031236-T01-IM6 | Soft Tissue Sarcoma | H1047R, E365K |
| P-0003178-T01-IM5 | Thyroid Cancer | H1047R, E542K |
| P-0032729-T02-IM6 | Thyroid Cancer | N345K, H1047Y |

The present disclosure next investigated potential patterns of co-mutation. In the majority of double PIK3CA mutant breast tumors, one of the mutations was either a helical or kinase domain major hotspot mutation (involving E542, E545, or H1047) (FIG. 10G), which are the most common alterations in single mutant tumors. The present disclosure performed codon enrichment analysis and determined that second-site E726, E453, and M1043 mutations were most significantly enriched in multiple mutant tumors compared to single mutant tumors in cBioPortal (FIG. 9B) and MSK-IMPACT (FIG. 9E) breast cancer datasets; this is compared to E542, E545, or H1047 mutations which were equally distributed between single and multiple mutant tumors. Almost all tumors containing second-site E726, E453, or M1043 mutations in cBioPortal (n=70 [88%]) and MSK-IMPACT (n=43 [100%]) also contained E542, E545, or H1047 mutations (FIG. 10G). In the non-breast cancer cohorts, E88 and E93 mutations were the most significantly enriched (FIG. 9B and FIG. 10D). Thus, the most frequent double PIK3CA mutant tumor combinations in breast cancer were comprised of a canonical “major mutant” hotspot (involving either E542, E545, or H1047) combined with a second “minor mutant” site (involving either E453, E726, or M1043) (FIG. 9F), and these recurrent mutational sites were specific to breast cancer compared to other cancer histologies.

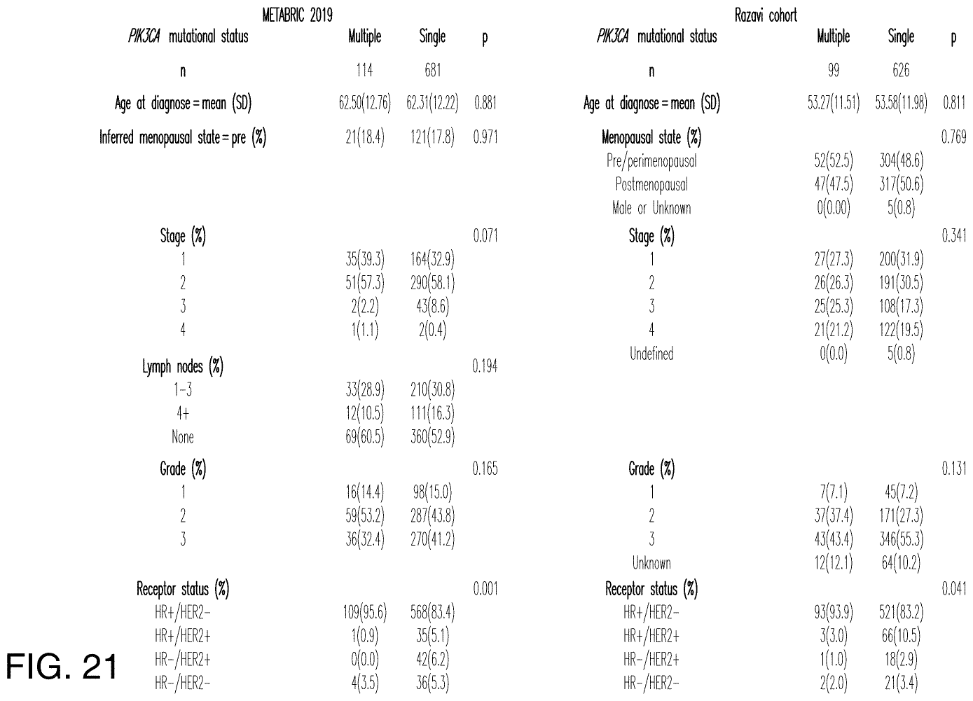

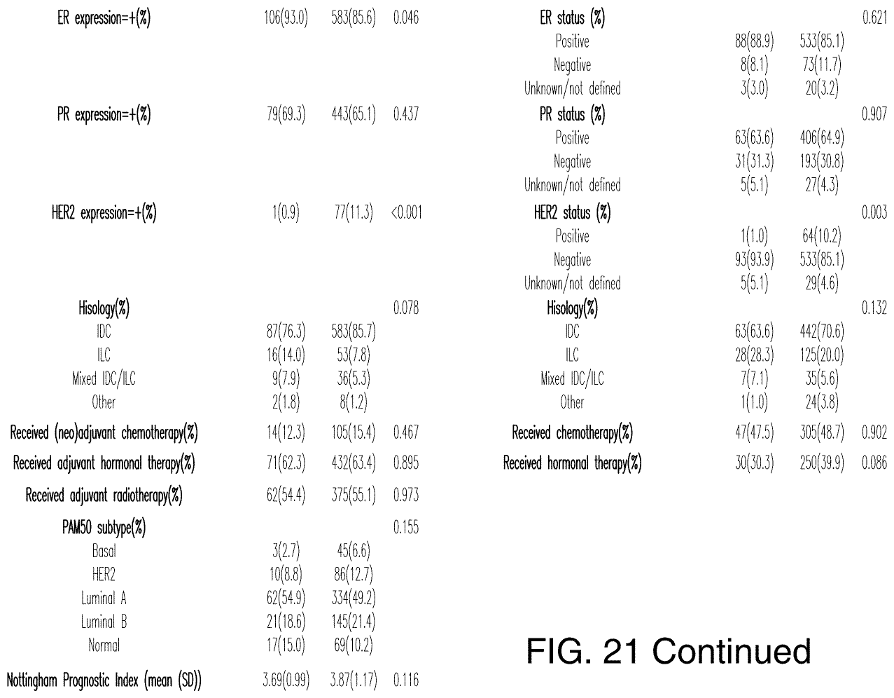

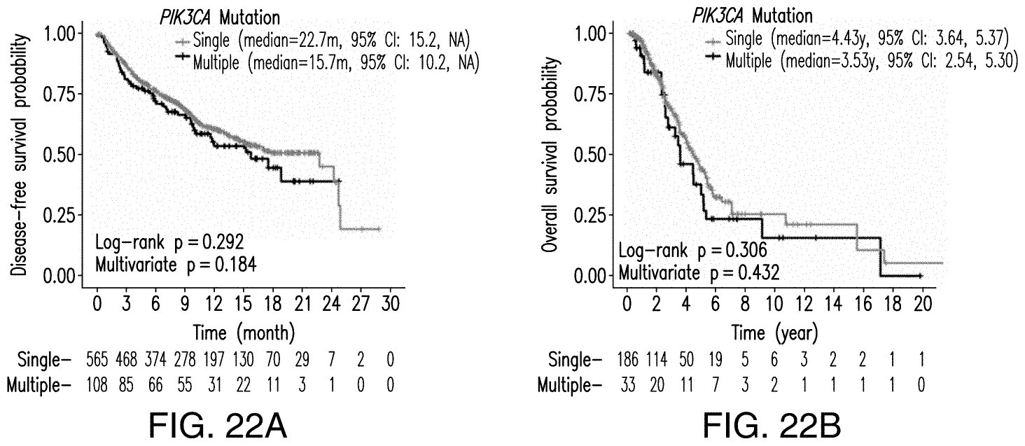

To determine if double mutants are in the same cell or are in different cells, the present disclosure analyzed clonality using FACETS (Shen et al., Nucleic Acids Res 44, e131 (2016)) of double mutant tumors from a large clinically-annotated breast cancer cohort (n=1918) (Ravazi et al., Cancer Cell 34, 427-438 e426 (2018)). Of the tumors that contained the most frequent double mutant combinations in breast cancer—E545K or H1047R major hotspots and E453, E726, or M1043 minor mutations (n=43)—most (64%) were clonal for both mutations (FIG. 9D). This was concordant with interpatient VAFs of multiple mutant breast tumors from cBioPortal (FIG. 10E), which follow a 1:1 linear distribution. The present disclosure performed additional clinicogenomic analysis of double PIK3CA mutant breast tumors from METABRIC 2019 (Bertucci et al., Nature 569, 560-564 (2019)) and Razavi 2018 cohorts (Razavi et al., Cancer Cell 34, 427-438 e426 (2018)), revealing that HER2 expression was less frequent with multiple vs single mutant breast tumors (FIG. 21) such that multiple PIK3CA mutations are enriched in hormone receptor-positive (HR+)/HER2− breast cancers, compared to other receptor subtypes (including HER2+ or triple negative breast cancers) (FIGS. 9E and 21). Notably, multiple and single mutations occur at similar frequencies in therapy-naïve primary and metastatic tumors (FIGS. 9E and 21). Invasive disease-free survival and overall survival are similar between multiple and single PIK3CA mutant patients on univariate and multivariate analyses (FIGS. 22A-22B).

### Double PIK3CA Mutations are in Cis on the Same Allele

Any two mutations in the same gene within a cell can be in cis on the same allele or in trans, on separate alleles. Since double PIK3CA mutations are most often clonal (in the same cell), establishing their allelic configuration is important as cis mutations would result in a single protein with two mutations while trans mutations would result in two proteins with separate individual mutations, and these could have different functional consequences.

To study the allelic configuration of double mutations the present example faced several technical hurdles based on the observation that the most frequent double PIK3CA mutants are located far apart in genomic DNA (FIG. 10F). An initial limitation was that tumor specimens are classically preserved as formalin fixed, paraffin embedded (FFPE) samples, which results in fragmented genomic DNA and RNA of ˜200 nucleotides, prohibiting phasing of recurrent double PIK3CA mutations (FIG. 11D). The present disclosure overcame this dependence by obtaining fresh frozen samples of patients known to carry two PIK3CA mutations in their tumors, by MSK-IMPACT. This could be done only for patients with metastatic disease (since most patients who underwent primary breast tumor resection had only FFPE samples available). In addition, even with fresh frozen tumor samples, current NGS library construction methods limit the allelic phasing of fragments to ˜300 nucleotides, again prohibiting this type of analysis for the most recurrent double PIK3CA mutations (FIG. 11D). To resolve this technical limitation, the present disclosure applied two alternative approaches. First, from initial double mutant (E545K/E726K) breast tumor with high VAF, the present disclosure performed bacterial colony Sanger sequencing and found that 14/14 (100%) of mutant cDNA inserts contained double mutations, in cis (FIG. 11A). The same technique was applied to the double PIK3CA mutant BT20 breast cancer cell line (P539R and H1047R) and found that 13/14 (92%) mutant cDNA inserts contained double mutations, in cis (FIG. 12A).

While Sanger sequencing of bacterial colonies can be used to determine the allelic configuration of double mutants, it is a heterologous system, exhibits low efficiency in biopsies with low cancer cell fraction, and is indirect for some double mutants far apart in the gene that require separate priming reactions. To solve these limitations, the present disclosure utilized single molecule real-time sequencing (SMRT-seq) (Eid et al, Science 323, 133-138 (2009)) (FIG. 11B), which uses long range sequencing of circular DNA templates, enabling direct phasing of the allelic configuration of all recurrent double PIK3CA mutants far apart in the gene. This is the first demonstration of SMRT-seq to phase recurrent mutations directly from solid tumor samples.

The present disclosure first analyzed BT20 cells as a control and three additional double PIK3CA mutant breast cancer cell lines with unknown allelic configurations: CAL148 (D350N/H1047R), MDA-MB-361 (E545K/K567R), and HCC202 (E545K/L866F) (FIG. 12B). While BT20 and CAL148 cell lines contain cis mutations, MDA-MB-361 cells contain trans mutations. HCC202 contains E545K and L866F mutations in trans, but also E545K and I391M mutations in cis. Thus, the present disclosure concluded that SMRT-seq is feasible to phase the allelic configuration of PIK3CA mutations and re-curate known cell line mutations.

Six patient tumor samples were used to demonstrate that the double mutations occur in cis, by SMRT-seq. This cohort contained samples from patients with E542K/E726K, E545K/E726K, E453K/H1047R, and E545K/M1043L double PIK3CA mutations, representative of the most frequent double mutants in breast cancer (FIG. 9F). All six patient tumors contained double mutations in cis (FIG. 11C).

The present disclosure also used next generation sequencing (NGS) by MSK-IMPACT (Table 6) and RNA sequencing (Table 7) on breast tumors from TCGA (N. Cancer Genome Atlas, Nature 490, 61-70 (2012)) to interrogate the allelic configuration of less frequent double PIK3CA mutants located close together in the gene. These findings support that double PIK3CA mutations are mainly found as cis mutations in breast cancer.

TABLE 6
Double PIK3CA mutant breast tumors phased as cis or trans mutants, by NGS

| Sample ID | Cancer Type | PIK3CA Mutation (gDNA) | Cis or trans |
|---|---|---|---|
| P-0012667-T01-IM5 | Breast Cancer | E385K + D390N | Cis |
| P-0001902-T01-IM3 | Breast Cancer | E542K + E545D | Cis |
| P-0014479-T01-IM6 | Breast Cancer | E542Q + E545K | Cis |
| P-0029802-T01-IM6 | Breast Cancer | E542K + E545K | Cis |
| P-0022021-T01-IM6 | Breast Cancer | E545K + D549H | Cis |
| P-0024420-T01-IM6 | Breast Cancer | E545K + D549N | Cis |
| P-0026885-T01-IM6 | Breast Cancer | M1004I + H1047R | Cis |
| P-0000107-T01-IM3 | Breast Cancer | D1017E + I1019V + Y1021H | Cis |
| P-0021201-T01-IM6 | Breast Cancer | L1036S + M1040T | Cis |
| P-0021040-T01-IM6 | Breast Cancer | H1047R + M1055L | Cis |
| P-0020645-T01-IM6 | Breast Cancer | M1043 + H1047R | Trans |

TABLE 7
Double PIK3CA mutant breast tumors phased
as cis mutants, by RNA-seq (TCGA)

| | | | Number | |
|---|---|---|---|---|
| | | | of reads | Number |
| | | PIK3CA | calling | of reads |
| | Cancer | mutations | both | spanning |
| Sample ID | Type | (RNA, in cis) | mutations | both loci |
| TCGA-AO- | Breast | 1004 + 1047 | 11 | 12 |
| A1KR-01A- | Cancer | | | |
| 12D-A142-09 | | | | |
| TCGA-A2- | Breast | 1047 + 1065 | 4 | 10 |
| A0EN-01A- | Cancer | | | |
| 13D-A099-09 | | | | |

### Double PIK3CA Mutations in Cis Hyperactivate PI3K and Enhance Proliferation

The present disclosure reasoned that the high frequency of double PIK3CA mutations in cis in breast cancer could reflect a selective advantage rather than being the result of randomly driven events. Taken individually, the minor PIK3CA mutations E453, E726, and M1043 demonstrated mild transforming activity in vitro as compared to the major mutations E542, E545, and H1047 (Zhang et al., Cancer Cell 31, 820-832 e823 (2017)). The present disclosure hypothesized that cis PIK3CA mutants demonstrate a hypermorphic function as they code for a single protein molecule with both major and minor mutations of varying activating capacities. The present disclosure therefore explored the effects of double PIK3CA mutations in cis on the activation of the PI3K pathway. E542K and E545K single hotspot mutants were predicted to have similar mechanisms of activation (Zhao et al., Proc Natl Acad Sci USA 105, 2652-2657 (2008)), and the present disclosure posited that mutations at the same amino acid position have also similar mechanisms. Thus, the present disclosure focused on the cis mutants E453Q/E545K, E453Q/H1047R, E545K/E726K, E726K/H1047R, and E545K/M1043L and their constituent single mutants for functional characterization.

The present disclosure stably overexpressed each cis mutant and constituent single mutant in MCF10A breast epithelial cells and NIH-3T3 fibroblasts, both of which have been previously used to characterize PIK3CA mutations, and also in MCF7 ER+ breast cancer cells engineered by somatic gene editing to carry a PIK3CA wildtype (WT) background (Isakoff et al., Cancer Res 65, 10992-11000 (2005); Ikenoue et al., Cancer Res 65, 4562-4567 (2005); Beaver et al., Clin Cancer Res 19, 5413-5422 (2013). Double PIK3CA mutations in cis increased downstream PI3K pathway signaling when compared to single hotspot mutants, as evidenced by increased phosphorylation of AKT (T308), AKT (S473), PRAS40, S6 (S235/236), and S6 (S240/244) under serum starvation in MCF10A cells (FIG. 13C), NIH-3T3 cells (FIG. 13D), and MCF7 cells (FIG. 14A). All cis mutants were capable of additional stimulation by growth factor, as shown by PDGF-BB or IGF1 stimulation of NIH-3T3 cells (FIG. 14B) though certain phosphoproteins were not further stimulated by growth factor (e.g. pS6 under IGF-1). Cis mutants prolonged downstream signaling kinetics as demonstrated by the E726K/H1047R MCF10A mutant which maintained increased phosphorylated AKT (T308 and S473) up to 48 hours (FIG. 1311). Cis mutants displayed increased proliferation by crystal violet assay in MCF10A cells as compared to single hotspot mutants (FIG. 13A and FIG. 13B). Cis mutations on the same allele were necessary for the increased signaling and growth phenotype, as E726K and H1047R in trans did not increase MCF10A cell signaling (FIG. 13H and FIG. 14C) and growth proliferation (FIG. 14D) more than single mutations.

The present disclosure next investigated whether cis mutant cells enhanced tumor growth in vivo compared to single mutants. NIH-3T3 nude mice allografts expressing the E726K/H1047R cis mutant demonstrated increased tumor growth compared to H1047R, or E726K (FIG. 13E) (Berenjeno et al., Nat Commun 8, 1773 (2017); Kinross et al., J Clin Invest 122, 553-557 (2012)). Of note, there was no difference in tumor growth between the single mutants and wild-type, supporting the notion that in some model systems single hotspot PIK3CA mutations are weakly oncogenic. In parallel to the enhanced tumorigenicity, and the observations in cell culture, E726K/H1047R cis mutant NIH-3T3 tumors exhibited higher activation of the PI3K pathway as shown by increased phosphorylation of AKT (S473) and AKT (T308) on western blotting (FIG. 13F) and AKT (S473) by immunohistochemistry (FIG. 13G).

### Double PIK3CA Mutations in Cis Combine Biochemical Effects of Single Mutants

p110α is constitutively bound to p85α, and this interaction stabilizes its structure, inhibiting catalytic activity (Yu et al., Mol Cell Biol 18, 1379-1387 (1998)). The prevailing model of PI3Kα activation occurs through the engagement of its p85α binding partner with phosphotyrosines on RTK signaling complexes. This interaction translates to a partial release of p85α from p110α which relieves catalytic inhibition (Burke et al., Proc Natl Acad Sci USA 109, 15259-15264 (2012)). Single oncogenic mutations recapitulate these events in distinct ways in the absence of phosphotyrosine binding, by weakening the interactions between p110α and p85α (mutants here describes as “disrupters”), or by binding to membrane (mutants here described as “binders”). The present disclosure structurally mapped the constitutive single mutants and postulated that E545K and E453Q act as disrupters while E726K, H1047R, and M1043L act as binders (FIGS. 15E-15F and FIGS. 16D-16F). Notably, none of these mutants is involved directly in the PI3Kα catalytic mechanism (Maheshwari et al., J Biol Chem 292, 13541-13550 (2017)). The present disclosure dissected the biochemical mechanisms by which these double PIK3CA mutations in cis increase PI3Kα activation, by purifying recombinant PI3Kα complexes containing single and double cis p110α mutations (FIG. 16B).

The present disclosure modelled cis mutant PI3Kα complex destabilization using thermal shift assays, which expose proteins to increasing levels of heat to determine the melting temperature. Unstable proteins will readily denature and aggregate at lower temperatures. p110α depends on its interaction with p85α to properly fold, and weakening their association renders them thermally labile (Yu et al., Mol Cell Biol 18, 1379-1387 (1998); Croessmann et al., Clin Cancer Res 24, 1426-1435 (2018)). All cis mutants tested demonstrated increased thermal instability as quantified by decreased melting temperatures, compared to each of their constituent minor and major mutants (FIG. 15A and FIG. 15H).

The present disclosure then measured basal recombinant kinase activity of using radioactive in vitro kinase assays, assessing for levels of radiolabeled 32P-PIP3 by thin-liquid chromatography (TLC). E453Q/E545K, E453Q/H1047R, and E545K/M1043L cis mutants demonstrated increased basal kinase activity compared to each of their constituent minor or major mutants (FIG. 15B and FIG. 15C).

To assess whether cis mutants increase lipid binding, the present disclosure performed liposome sedimentation assays with liposomes containing anionic lipids (modeled after the inner leaflet of the plasma membrane) with and without 0.1% PIP2 (the physiologic concentration) given differential contributions to lipid binding to PI3K (Hon et al., Oncogene 31, 3655-3666 (2012)). All cis mutants tested exhibited increased binding to anionic liposomes compared to single major or minor mutants (FIGS. 15D, 15I), with E453Q/E545K, E453Q/E1047R, E545K/E726K, and E726K/H1047R cis mutants showing enhanced binding to PIP2 liposomes compared to their constituent single mutants.

### Double PIK3CA Mutations in Cis are Hypersensitive to PI3K Inhibition in Cells

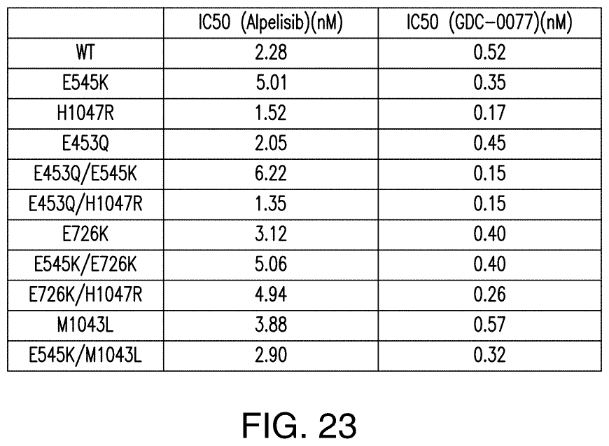

The biochemical and functional data herein presented suggested that double PIK3CA mutants in cis resulted in a constitutive activation of PI3K signaling, implying that cells bearing these mutations were more dependent on the PI3K pathway for proliferation and survival. IC50 values for the PI3Kα inhibitors alpelisib and GDC-0077 are similar among the recombinant single and cis mutant PI3Kα complexes (FIG. 23). Similar phenomena have been observed with other oncogenes, where both wild-type and translocated/mutant proteins are inhibited at clinically attainable drug concentrations (Druker et al., Nat Med 2, 561-566 (1996); Sharma et al., Genes Dev 21, 3214-3231 (2007)).

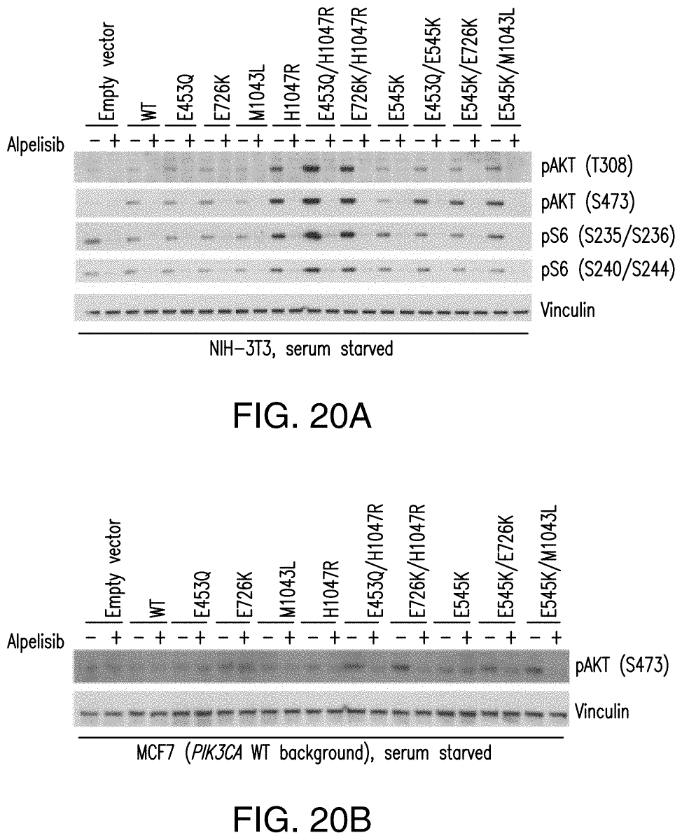

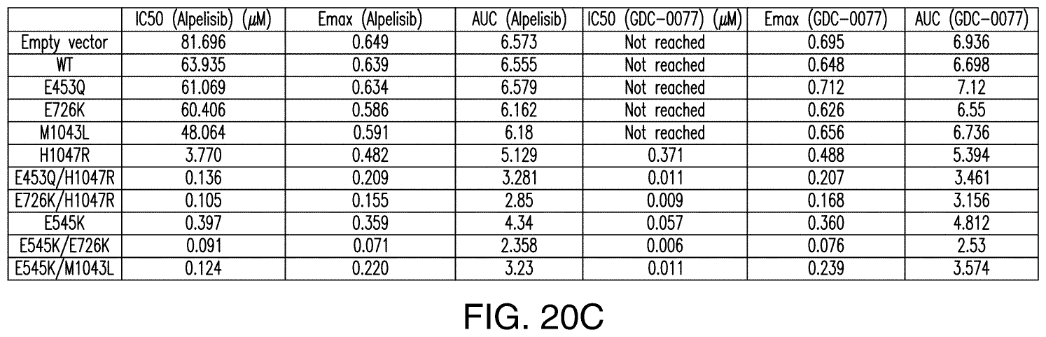

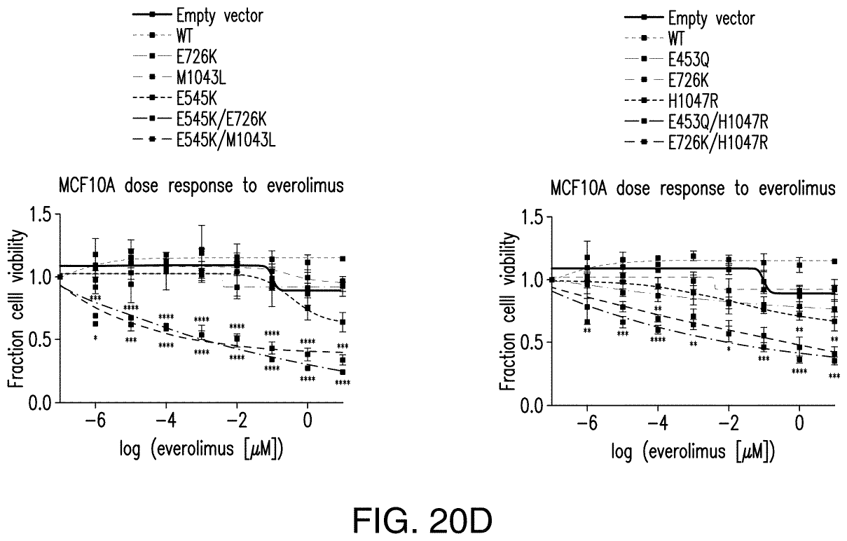

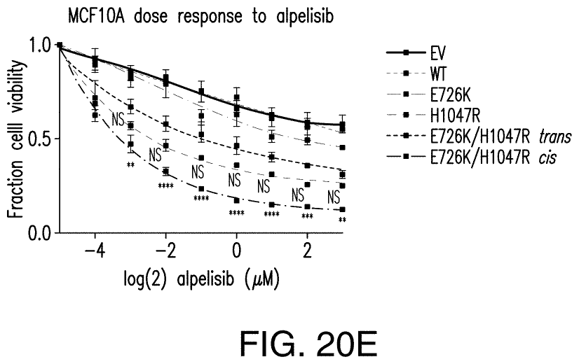

Therefore, the present disclosure tested whether cis mutant cells exhibit differential sensitivity to PI3Kα inhibitors. While in the absence of pharmacological pressure cis mutant signaling was increased compared to single mutants, treatment with the PI3Kα inhibitors alpelisib or GDC-0077 (Fallahi-Sichani et al., Nat Chem Biol 9, 708-714 (2013)) resulted in a similar inhibition of phosphorylated AKT (T308 and S473), S6 (S235/236), and S6 (S240/244) among all the MCF10A clones (FIG. 6D and FIG. 6E). Similar results were obtained in NIH-3T3 cells (FIG. 20A) and MCF7 cells (FIG. 20B). The present disclosure then used the MCF10A cell culture models to test cell growth upon PI3Kα inhibition. E545K and H1047R major hotspot mutants were more sensitive to alpelisib (FIG. 6F) and GDC-0077 (FIG. 6G) compared to minor mutants and WT. In turn, all cis mutants were more sensitive to alpelisib and GDC-0077 compared to the E545K or H1047R major hotspots (FIGS. 6F-6G) with respect to IC50, Emax, and area under the curve (AUC) (Singh et al., Ann Diagn Pathol 17, 322-326 (2013)) (FIG. 20C). Cis mutants were also more sensitive to downstream PI3K pathway inhibitors including everolimus (FIG. 20D), compared to single mutants. In contrast, mutations in trans were less sensitive to alpelisib compared to cis mutants and were no more sensitive than the single major mutant, as demonstrated by E726K/H1047R (FIG. 20E). IC50 values for recombinant cis mutant kinases were not different from single mutants.

### Multiple PIK3CA Mutant Tumors are Hypersensitive to PI3K Inhibition in Patients

The present disclosure investigated the effects of multiple PIK3CA mutations on clinical response to PI3Kα inhibitors in metastatic breast cancer. The present disclosure analyzed response data from SANDPIPER, a phase III registrational clinical trial that tested the efficacy of the PI3Kα inhibitor taselisib (GDC-0032), with fulvestrant (an estrogen receptor [ER] degrader) in metastatic ER-positive PIK3CA mutant breast cancer. This is the largest randomized clinical study testing a PI3Kα inhibitor (631 patients).

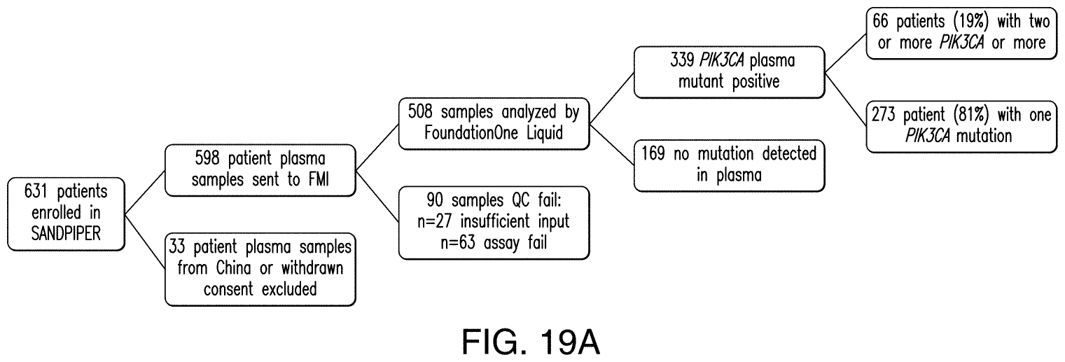

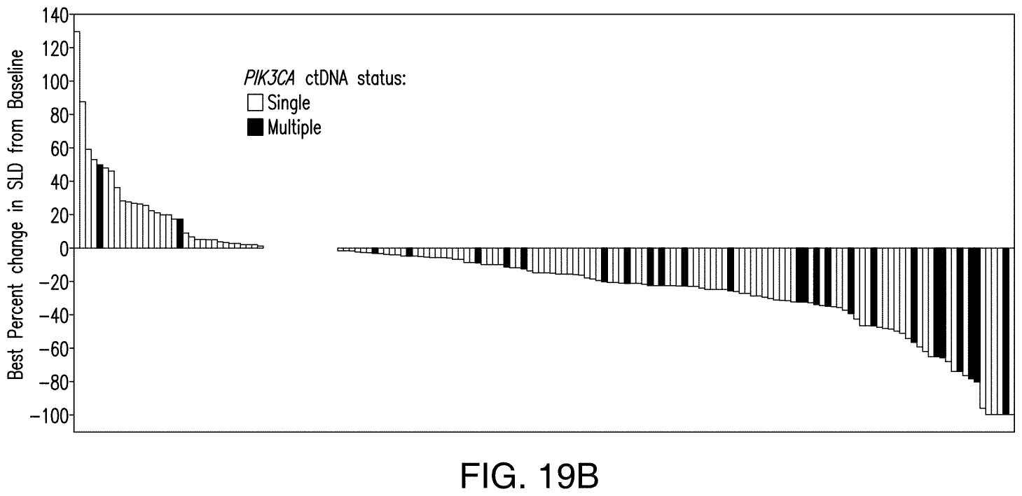

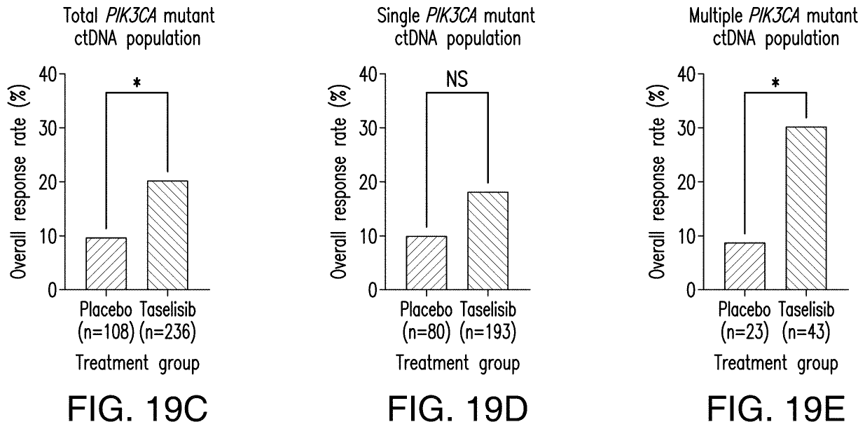

Many patients with metastatic ER-positive breast cancer enrolled in this trial had bone metastases, which must be decalcified to be analyzed by NGS, which render DNA sequencing particularly challenging (Singh et al., Ann Diagn Pathol 17, 322-326 (2013)). Thus, the present disclosure used circulating tumor DNA (ctDNA), which has been utilized in many breast cancer clinical trials (Baselga et al., Lancet Oncol 18, 904-916 (2017); Andre et al., N Engl J Med 380, 1929-1940 (2019); Baselga et al., N Engl J Med 366, 520-529 (2012); Turner et al., N Engl J Med 373, 209-219 (2015)), to detect the presence of multiple mutations. Of the 631 patients on the trial, 598 had plasma samples available for analysis, of which 508 were adequate for testing (FIG. 19A). Samples were tested using the Foundation One liquid assay, which sequences the entire PIK3CA gene. Of the 339 patients with detected PIK3CA mutations, 66 (19%) had 2 or more PIK3CA mutations. Notably, this is even higher than the frequency observed of archival tumor testing (12%) and may reflect the ability of ctDNA to detect global tumoral heterogeneity vs tumor biopsy of a single site.

Individual PIK3CA mutant patient responses on the taselisib arm were denoted on the waterfall plot (FIG. 19B), where more mutant patients exhibited tumor shrinkage than tumor growth. The present disclosure examined differences in overall response rates, defined as tumor shrinkage >30%. PIK3CA mutant patients on the taselisib arm (n=236) had an overall response rate of 20.3% vs 9.7% compared to the placebo arm (n=103) (95% CI 15.5-25.9% vs 4.8-16.7%, p=0.0202) (FIG. 19C). This result confirmed that the presence of PIK3CA mutations predicts response to PI3Kα inhibition (Baselga et al., Lancet Oncol 18, 904-916 (2017); Andre et al., N Engl J Med 380, 1929-1940 (2019); Azambuj a et al., Journal of Clinical Oncology 33, no. 15_suppl; Di Leo et al., Lancet Oncol 19, 87-100 (2018)).

The present disclosure then compared responses of patients with single vs multiple mutations. Patients with multiple mutant tumors experienced tumor shrinkage more than patients with single mutant tumors (FIG. 19B). Single PIK3CA mutant patients on the taselisib arm (n=193) had an overall response rate of 18.1% vs 10.0% compared to the placebo arm (n=80) (95% CI 13.0-24.2% vs 4.4-18.1%, p=0.0981) (FIG. 19D). However, multiple PIK3CA mutant patients on the taselisib arm (n=43) had an overall response rate of 30.2% vs 8.7% compared to the placebo arm (n=23) (95% CI 18.4 44.9% vs 1.6-26.8%, p=0.0493) (FIG. 19E).

### Postulated Biochemical Mechanisms of PIK3CA Mutations

E545K and E453Q (Mandelker et a., Proc Natl Acad Sci USA 106, 16996-17001 (2009); Miller et al., Oncotarget 5, 5198-5208 (2014)) are located in the binding interfaces between p110α and p85α and are predicted to be disrupters. E545K, located in the helical domain, disrupted binding to the p85α nSH2 domain and had a similar outcome to phosphotyrosine peptide binding to p85α (FIGS. 15E-15F), and E453Q impaired p110α C2 domain binding to the p85α iSH2 domain (FIGS. 15E-15F). The orientations of p110α C2 to p85α iSH2 were similar in the WT, WT+PIP2, and H1047R structures, with root mean square deviation (RMSD) values <1 Å (FIG. 16E); however, there were subtle changes in the C2 loop regions interacting with p85α iSH2 including the orientation of E453 which may be functionally relevant (FIG. 16E) (Mandelker et a., Proc Natl Acad Sci USA 106, 16996-17001 (2009); Miller et al., Oncotarget 5, 5198-5208 (2014); Science 318, 1744-1748 (2007)). H1047R and M1043L are located along the C-terminal tail, which forms part of the membrane-docking surface and are therefore predicted to be binders (FIGS. 15E-15F). Structurally, H1047R is postulated to increase membrane binding through interactions of the mutated arginine as well as reorganization of a C-terminal loop that also interacts with membrane. E726K is in the kinase domain and has been reported to be activating, but its mechanism is unknown (Zhang et al., Cancer Cell 31, 820-832 e823 (2017)). In crystal structures (Mandelker et a., Proc Natl Acad Sci USA 106, 16996-17001 (2009); Miller et al., Oncotarget 5, 5198-5208 (2014); Science 318, 1744-1748 (2007)), E726 was located in the membrane binding interface (FIG. 16C and FIG. 16D) and was oriented outwards directed towards the membrane (FIG. 16F). Therefore, the present disclosure hypothesized that E726K is also a binder, as the mutant lysine would increase positive charge and promote binding to the negatively charged phospholipids at the plasma membrane (FIG. 13D and FIG. 16F).

### Rationale for Recombinant Protein Purification Strategy

Recombinant full-length human PI3Kα complexes were purified from suspension EXPI293 human embryonic kidney cells (FIG. 16A and FIG. 16B). Fusing affinity tags to the termini of PIK3CA altered its basal catalytic activity (Sun et al., Cell Cycle 10, 3731-3739 (2011)). Structurally, the N-terminus sits along its binding interface with p85α and the C-terminus is located near its catalytic site. To generate recombinant p110α in its most native form, the present disclosure developed a purification scheme that utilizes a polyhistidine tag on the N-terminus p85α to purify untagged p110α, as a heterodimeric complex.

### DISCUSSION

In this work, the present disclosure identified double mutations in cis as a novel genomic alteration in PIK3CA, the most frequently mutated oncogene in human cancer (Kandoth et al., Nature 502, 333-339 (2013)). Double PIK3CA mutations in cis activated PI3K pathway cellular signaling and promoted growth more so than single mutants, through a combination mechanism of increased membrane binding and increased p85α disinhibition. The overall consequence of these cis mutations was a phenotype of enhanced oncogenicity and greater response to PI3Kα inhibitors compared to single mutations, in preclinical models and in the largest randomized clinical trial testing a PI3Kα inhibitor in breast cancer patients.

While cancers can accumulate numerous mutations in functionally relevant genes, many tumors depend on one gene to maintain the malignant phenotype, which has led to the concept of oncogene addiction. Oncogene addiction forms the rationale for the clinical development of many targeted therapies that have altered the natural history of human cancer (Weinstein et al., Clin Cancer Res 3, 2696-2702 (1997); Slamon et al., N Engl J Med 344, 783-792 (2001); Druker et al., N Engl J Med 344, 1031-1037 (2001); Lynch et al., N Engl J Med 350, 2129-2139 (2004)). While there are no formal definitions for oncogene addiction, some critical tenets are that the altered oncogene is sufficient for growth, and that inactivation of the oncogene induces tumor regression in both preclinical and clinical models (Weinstein et al., Nat Clin Pract Oncol 3, 448-457 (2006)). The herein presented findings that PIK3CA double mutations in cis synergistically increased growth and sensitivity to PI3Kα inhibition compared to single mutations implicate a model of oncogene addiction to mutant PIK3CA in breast cancer.

The common practice of sequencing only certain single nucleotide variants or some but not all exons across a gene likely underestimated the frequency of multiple mutations in PIK3CA mutant cancers at <1% (Saal et al., Cancer Res 65, 2554-2559 (2005); Yuan et al., Oncogene 27, 5497-5510 (2008)); in fact the true frequency is ˜10-15% which translates into a clinically meaningful number of patients who may derive additional benefit from targeted therapy. PI3Kα inhibitors are now a standard of care in PIK3CA-mutant ER+ metastatic breast cancer and are being explored in other PIK3CA mutant tumor histologies (Jhaveri et al., Cancer Research 78, CT046-CT046 (2018)). The herein presented findings provide a rationale for the selection of PI3Kα inhibitors in earlier therapeutic settings for multiple PIK3CA mutant metastatic breast cancer patients, and for the design of clinical trials testing the efficacy of PI3Kα inhibitors in patients with multiple PIK3CA mutant tumors.

### Materials and Methods

Mutational Data

All cases reported with PIK3CA mutation were downloaded from www.cbioportal.org on Sep. 18, 2018. Ten breast cancer studies were analyzed within the Breast Cancer cohort. Those cases not found in METABRIC and TCGA were combined as Breast Cancers (others). Cell line and xenograft studies were removed in Breast and Pan Cancer cohorts.

The MSK IMPACT dataset consisted of 28139 tumor samples from patients who were prospectively sequenced as part of their active care at Memorial Sloan Kettering Cancer Center (MSKCC) between January 2014 and September 2018, as part of an Institutional Review Board-approved research protocol (NCT01775072). All patients provided written informed consent, in compliance with ethical regulations. The details of patient consent, sample acquisition, sequencing and mutational analysis have been previously published (Zehir et al., Nat Med 23, 703-713 (2017)). Briefly, matched tumor and blood specimens for each patient were sequenced using Memorial Sloan Kettering-integrated mutation profiling of actionable cancer targets (MSK-IMPACT)—a custom hybridization capture-based next-generation sequencing assay (Cheng et al, J Mol Diagn 17, 251-264 (2015)). All samples were sequenced with 1 of 3 incrementally larger versions of the IMPACT assay, including 341, 410, and 468 cancer-associated genes, respectively. All PIK3CA mutations were identified and tumors were identified as containing single, double, or multiple PIK3CA mutations.

Codon Enrichment Analysis

PIK3CA single and double mutant tumors were combined in the indicated cohorts.

Tumors were analyzed for the frequency of a particular amino acid site mutation across the whole p110α protein in double mutant tumors versus single mutant tumors, compared to chance, as assessed by Fisher's exact test (two-tailed). Statistics were calculated together for all studies.

Phasing Mutations and Clonality Analysis

To determine the allelic configuration of multiple somatic mutations in the same gene and tumor, the present disclosure implemented a computational framework for read-backed phasing. To this end, the present disclosure exploited the fact that if two mutations were near enough in genomic position to be spanned by the same sequencing reads, then the identification of individual sequencing reads calling both variants at once unambiguously indicated that the different variants arose on the same DNA fragment, and therefore were in cis in the tumor genome. Conversely, if a large proportion of the reads spanning both mutations' loci called either mutation, but none call them both, and the two mutations were clonal enough to have arisen in the same cells, this implied that the two mutations arose in trans. Briefly, when two or more mutations in the same gene were found in a sample in the tumor sequencing dataset, the tumor's raw sequencing data in BAM format was algorithmically queried using Samtools (version 1.3.1) (Li et al., Bioinformatics 25, 2078-2079 (2009)) for the reads mapping to the loci of each mutation in that gene. The unique barcodes for the individual read-pairs calling each mutant allele were then obtained using the sam2tsv function from jvarkit (Lindenbaum P. (2015) JVarkit: java-based utilities for bioinformatics. FigShare, doi:10.6084/m9.figshare.1425030). By inspecting the barcodes calling the different mutant alleles in a gene, the present disclosure called two mutations in cis if both mutations were called by the same read-pair (in at least two distinct read-pairs, to mitigate false positives due to sequencing error). Conversely, the present disclosure called two mutations in trans if their loci were spanned by at least 10 reads, but less than two called them both at once, and their cancer cell fractions (as estimated by the FACETS algorithm (version 0.3.9)) (Shen et al., Nucleic Acids Res 44, e131 (2016))summed to at least 100%, indicating that they likely arose in the same cancer cells. FACETS was also used for clonality analyses on double mutant tumors.

Fresh Frozen Tumor Acquisition

Patients were initially identified as having double PIK3CA mutant tumors by MSK-IMPACT on FFPE samples, then were consented for collection of fresh tumor biopsies.

RNA Extraction and cDNA Generation

RNA was extracted from cell pellets (1×10⁷ cells) using the RNeasy Mini Kit (Qiagen), as specified by the manufacturer. Briefly, cells were homogenized in 350 μL lysis buffer (buffer RLT) by needle shearing, passing the resuspended pellet through a 20-gauge needle attached to a 5 mL syringe 10 times until a homogenous lysate was achieved. RNA extract from the lysate was then mixed with 70% ethanol and applied to the RNeasy spin column. Following the designated binding and wash steps, total RNA was eluted from the column twice using 30 μL RNase free water for each elution, resulting in 60 μL extracted RNA per sample. Upon extraction, total RNA was aliquoted and stored at −80° C. for later use. Total cDNA for SMRT-seq was generated using the SuperScript IV First Strand Synthesis System for RT-PCR using 5 μL total RNA input, the provided oligo (dT) to prime first-strand synthesis, and according to the manufacturer's protocol. Aliquots of cDNA were stored at −20° C. until needed for custom-primer, targeted PIK3CA amplification to achieve full-length molecules to phase variants of interest for diagnostic purposes. Total cDNA for Sanger sequencing was generated using the iScript cDNA Synthesis Kit (Bio-Rad).

Sanger Sequencing

BT20, CAL148, HCC202, and MDA-MB-361 cells were purchased from ATCC. Fresh frozen tumors and samples were homogenized in RIPA buffer supplemented with protease and phosphatase inhibitors. Full length PIK3CA cDNA was amplified using Taq polymerase to generate 3′ A-tailed fragments and purified using a Qiaquick Gel Extraction kit (Qiagen). Full length PIK3CA cDNA was ligated into pGEM-T (Promega), transformed into E. coli, and plated on LB plates containing ampicillin, IPTG, and X-Gal for blue and white colony selection. White colonies were selected, miniprep plasmid DNA was isolated (Qiagen), and were submitted for Sanger sequencing.

PIK3CA Amplification for SMRT-Seq

Targeted PIK3CA amplification was performed using polymerase chain reaction (PCR) with High Performance Liquid Chromatography (HPLC)-purified SMRT-seq primers. SMRT-seq primerswere:

Forward:
[Seq ID No: 1]
TGGGACCCGATGCGGTTA
Reverse:
[Seq ID No:2]
AATCGGTCTTTGCCTGCTGA

The primers were synthesized at Integrated DNA Technologies, purified, and diluted to 10 μM in 0.1×TE buffer before use. Each reaction totaled 50 μL and consisted of 5 μL total cDNA, 5 μL 10×LA PCR Buffer II (Mg2+ plus), 8 μL of 2.5 mM dNTP mix, 2 μL each of PIK3CA-F and PIK3CA-R, 27.5 μL of nuclease free water, and 0.5 μL of LA-Taq polymerase (part no. RRO2C, TaKaRa Bio). Reactions were heated to 98° C. for 3 minutes and then subjected to 32 cycles of PCR using the following parameters: 25-sec denaturation at 98° C., followed by 15-sec annealing at 55° C., followed by 8-min extension at 68° C. After the 32nd cycle, the reactions were incubated for 15 min at 68° C. and then held at 4° C. PIK3CA amplicons were purified from PCR reactions using 1×AMPure PB beads, as described by the manufacturer (part no. 100-265-900, Pacific Biosciences). PIK3CA amplicons were visualized and quantified using the 2100 Bioanalyzer System with the DNA 12000 kit (Agilent Biosciences).

SMRTbell Library Preparation and Sequencing

SMRTbell template libraries of the ˜3.3-kb PIK3CA amplicon insert size were prepared according to the manufacturer's instructions using the SMRTbell Template Prep Kit 1.0 (part no. 100-259¬100; Pacific Biosciences). A total of 250 ng of purified PIK3CA amplicon was added directly into the DNA damage repair step of the Amplicon Template Preparation and Sequencing protocol. Library quality and quantity were assessed using the DNA 12000 Kit and the 2100 Bioanalyzer System (Agilent), as well as the Qubit dsDNA Broad Range Assay kit and Qubit Fluorometer (Thermo Fisher). Sequencing primer annealing and P6 polymerase binding were performed using the recommended 20:1 primer:template ratio and 10:1 polymerase:template ratio, respectively. SMRT sequencing was performed on the PacBio RS II using the C4 sequencing kit with magnetic bead loading and one-cell-per-well protocol and 240-minute movies.

SMRT-Seq Haplotype Generation and Variant Calling

To generate haplotypes and identify variants, data were processed by the Minor Variants Analysis Tool as part of the SMRTLink 5.1 bioinformatics suite (Pacific Biosciences) using NM_006218.3, the NCBI Reference Sequence for PIK3CA. Briefly, circular consensus sequence (CCS) reads were generated and filtered on reads that were ≥99.9% (Q30) accurate as input for haplotype and variant analysis. A conservative 5% variant frequency threshold was also applied, such that the phased haplotypes were generated using variants called with very high confidence. Phased haplotypes indicated those variants that were present in cis- or trans- within each selected sample.

Mutagenesis and Cloning

The present disclosure cloned PIK3CA without affinity tags, as N-terminal tags artificially increase kinase activity and C-terminal tags may interfere with membrane binding (Sun et al., Cell Cycle 10, 3731-3739 (2011); Hon et al., Oncogene 31, 3655-3666 (2012)). For pBabe puro HA PIK3CA and pcDNA 3.4-PIK3CA, the SNP coding for I143V was mutated back to the WT isoleucine by site-directed mutagenesis. For pBabe puro HA PIK3CA, the N-terminal HA tag was deleted by site-directed mutagenesis. For pDONR223_PIK3CA_WT, a C-terminal stop codon was inserted by site-directed mutagenesis. In total, these modifications resulted in untagged WT PIK3CA in the various plasmids. Onto these WT backbones, E545K and H1047R mutants were cloned. After this first round of mutagenesis, E453Q, E726K, and M1043L were cloned into the E545K and H1047R plasmids to create double cis mutants. pDONR plasmids were recombined with the pLX-302 acceptor plasmid using Gateway LR Clonase II Enzyme mix (Thermo Fisher). Plasmid backbone mutagenesis primers were:

PIK3CA-WTC-terminal stop codon (pDONR223)
Forward:
[Seq ID No: 13]
CATGCATTGAACTGATTGCCAACTTTC
Reverse:
[Seq ID No: 14]
GAAAGTTGGCAATCAGTTCAATGCATG
PIK3CA V143I to WT isoleucine
Forward:
[Seq ID No: 17]
GACTTCCGAAGAAATATTCTGAACGTTTGTAAA
Reverse:
[Seq ID No: 18]
TTTACAAACGTTCAGAATATTTCTTCGGAAGTC
PIK3CA N-terminal HA tag removal
(pBabe puro Myr HA PIK3CA)
Forward:
[Seq ID No: 15]
GATCCAAGCTTCACCATGCCTCCAAGACCATCATCA
Reverse:
[Seq ID No: 16]
TGATGATGGTCTTGGAGGCATGGTGAAGCTTGGATC

PIK3CA mutagenesis primers were:

E545K
Forward:
[Seq ID No: 3]
CCTCTCTCTGAAATCACTAAGCAGGAGAAAGATTTTC
Reverse:
[Seq ID NO:4]
GAAAATCTTTCTCCTGCTTAGTGATTTCAGAGAGAGG
H1047R
Forward:
[Seq ID No: 5]
CAAATGAATGATGCACGTCATGGTGGCTGGAC
Reverse:
[Seq ID No: 6]
GTCCAGCCACCATGACGTGCATCATTCATTTG
E453Q
Forward:
[Seq ID No: 7]
CCAGTACCTCATGGATTACAGGATTTGCTGAACCCTATTG
Reverse:
[Seq ID No: 8]
CAATAGGGTTCAGCAAATCCTGTAATCCATGAGGTACTGG
E726K
Forward:
[Seq ID No: 9]
GAGAAGAAGGATAAAACACAAAAGGTAC
Reverse:
[Seq ID No: 10]
GTACCTTTTGTGTTTTATCCTTCTTCTC
M1043L
Forward:
[Seq ID No: 11]
GTATTTCATGAAACAACTGAATGATGCACATCATGGTGGCTGGAC
Reverse:
[Seq ID No:12]
GTCCAGCCACCATGATGTGCATCATTCAGTTGTTTCATGAAATAC

Cell Lines, Retroviral, and Lentiviral Production, and Drugs

NIH-3T3 cells were maintained in DMEM media supplemented with 10% FCS and 1% Pen/Strep. MCF-10A cells were maintained in DF-12 media supplemented with 5% filtered horse serum (Invitrogen), EGF (20 ng/μL) (Sigma), hydrocortisone (0.5 mg/mL) (Sigma), cholera toxin (100 mg/mL) (Sigma), insulin (10 μg/mL) (Sigma), and 1% penicillin/streptomycin. MCF7 cells and 293T cells were maintained in DMEM media supplemented with 10% FBS and 1% Pen/Strep. Cells were used at low passages and were incubated at 37° C. in 5% CO2.

For retroviral and lentiviral production, 7×106 293T cells were seeded in 10-cm plates and transfected with the plasmid of interest, pCMV-VSVG, and pCMV-dR8.2 (for lentivirus) using Jetprime (Polyplus Transfection). Viruses were harvested 48 hours after transfection and were filtered through a 0.45 μm filter (Millipore). Target cells were infected using fresh viral supernatants and were selected using puromycin (2 μg/mL) to obtain stable clones. For trans mutants, a 1:1 ratio of viruses was infected. Cell lines were genotyped to confirm the presence of the PIK3CA cDNA sequence. Alpelisib was purchased (Selleck). GDC-0077 was obtained on MTA from Genentech.

Cell Proliferation Assays

MCF10A cell lines were seeded in serum starved media (MCF10A media without EGF or insulin), at 10000 cells/mL in 12 well plates. Cells were grown, and time points were collected daily from 0-4 days and fixed in formalin. Formalin fixed cells were developed using crystal violet and pictures were taken for day 4 growth. Acetic acid was added and OD595 was obtained. OD values were normalized to day 0 for each cell lines and plotted.

Western Blotting

MCF10A, NIH-3T3 cells, and MCF7 cells were seeded in normal growth medium, either 4 million cells in 10 cm dishes or 400000 cells in 6 cm plates. 24 hours later, cells were washed twice with PBS then refreshed with serum starved media. Serum starved media for MCF10A cells used MCF10A media with 5% horse serum and without EGF or insulin. Serum starved media for NIH-3T3 and MCF7 cells used 0.1% FCS and 0.1% FBS, respectively. For growth factor stimulation experiments, PDGF-BB (20 ng/mL) was added for 30 minutes, and IGF-1 (10 nM) was added for 10 minutes, after serum starvation. For drugging experiments cells were washed twice with PBS then refreshed with serum starved media with DMSO or 1 μM alpelisib or 62.5 nM GDC-0077 (the IC50 [GDC-0077] of MCF10A E545K cells per FIG. 6G) for the indicated time points. Cells were washed with PBS twice, and lysed in RIPA buffer supplemented with protease and phosphatase inhibitors (Roche). Allograft tumor samples were also lysed in RIPA buffer supplemented with protease and phosphatase inhibitors. Protein extracts were quantified and normalized (NuPage), separated using SDS-PAGE gels, and transferred to PVDF membranes. All primary antibodies were diluted 1:1000 and anti-rabbit IgG secondary antibody (GE Healthcare) (1:4000) was used. Membranes were probed using specific antibodies. p110α (#4249), pAKT (S473) (#4060), pAKT (T308) (#13038), total AKT (#4691), pPRAS40 (#13175), pS6 (240/244) (#5364), pS6 (235/236) (#4858), total S6 (#2217), pERK1/2 (T202/Y204) (#4370), total ERK (#4695), and vinculin (#13901) were purchased from Cell Signaling Technology (CST). All primary antibodies were diluted 1:1000 and anti-rabbit IgG secondary antibody (GE Healthcare) (1:4000) was used. For quantification, densitometry was performed using ImageJ (Isakoff et al., Cancer Res 65, 10992-11000 (2005).

Mouse Allografts

5×10⁶ NIH-3T3 cells in 1:1 PBS/Matrigel (Corning) were injected subcutaneously into six-week-old female athymic nude mice. When tumors reached a volume of ˜150 mm³, mice measured twice a week during a month. 4 tumors per group were used in these studies. For statistical analysis, outliers were removed using Grubbs' test (α=0.05). Tumors were harvested at the end of the experiment, fixed in 4% formaldehyde in PBS, and paraffin-embedded. IHC was performed on a BOND RX processor platform (Leica) using standard protocols with BOND Epitope Retrieval Solution 2 (Leica). Primary staining with pAKT (S473) (D9E), 1:100 (CST) for 30 minutes was followed by staining with a Bond Polymer Refine Detection kit (Leica) for 60 minutes. Studies were performed in compliance with MSKCC institutional guidelines under an IACUC approved protocol. The animals were immediately euthanized as soon as investigators were notified that the tumors reached the IACUC set limitations.

Structural Mapping

PI3K structural mapping was performed on PDB 2RD0, 3HHM, and 4OVU using PyMOL (Schrodinger, LLC, in The PyMOL Molecular Graphics System, Version 1.8. (2015)).

Protein Expression and Purification

EXPI-293F cells (Thermo Fisher) were incubated at 37° C. in 8% CO2, in spinner flasks on an orbital shaker at 125 rpm in Expi293 Expression Medium (Thermo Fisher). 300 μg of pcDNA 3.4-PIK3CA and 200 μg pcDNA 3.4-PIK3R1 were combined and diluted in Opti-MEM I Reduced Serum Medium (Thermo Fisher). ExpiFectamine 293 Reagent (Thermo Fisher) was diluted with Opti-MEM separately then combined with diluted plasmid DNA for 10 minutes at room temperature. The mixture was then transferred slowly to 500 mL EXPI-293F cells (3×10⁶ cells/mL) and incubated. 24 hours later, ExpiFectamine 293 Transfection Enhancer 1 and Enhancer 2 (Thermo Fisher) were added. Cells were harvested 3 days after transfection and centrifuged at 4000 rpm for 30 minutes and frozen at −20° C.

All steps of protein purification were performed at 4° C. Cell pellets were solubilized in lysis buffer (50 mM Tris pH 8.0, 400 mM NaCl, 2 mM MgCl2, 5% glycerol, 1% Triton X-100, 5 mM β-mercaptoethanol, 20 mM imidazole) supplemented with EDTA-free protease inhibitor (Sigma) and lysed using a Dounce homogenizer for 20 strokes. Lysates were centrifuged at 14000 rpm for 60 minutes and clarified lysates were affinity purified on Ni-NTA resin (Qiagen) by batch binding at 4° C. for 1 hour. Resin was washed with 10 column volumes of lysis buffer (50 mM Tris pH 8.0, 500 mM NaCl, 2 mM MgCl2, 2% glycerol, 20 mM imidazole) and eluted in 10 column volumes of elution buffer (50 mM Tris pH 8.0, 100 mM NaCl, 2 mM MgCl2, 2% glycerol, 1 mM TCEP, 250 mM imidazole). Eluted protein was buffer exchanged with elution buffer without imidazole, concentrated using 100 kDa Ultra Centrifugal Filter Units (Amicon), and flash frozen in liquid nitrogen with 20% glycerol. Concentrations of PI3K complexes used in all biochemistry experiments were normalized by Western blotting for p110α as compared to 1 WT PI3K complex.

Thermal Shift Assays

1 μg of PI3K complex was added to 10 μL 5× Assay Buffer I (SignalChem), 2 μL 1 mM ATP, and 1 μL BSA (2 mg/mL) and distilled water to a total volume of 50 μL into each tube of a MicroAmp Optical 8-Cap strip (Thermo Fisher) at room temperature. For each experiment, one 8-cap strip was prepared per PI3K construct. Tubes were placed in a C1000 Touch Thermocycler (BioRad). Samples were cycled at 46° C. for 30 seconds, then on a temperature gradient from 46°-61.7° C. for 3 minutes, then 25° C. for 3 minutes. Samples were spun in a Minispin centrifuge for 30 seconds and 40 μL of the supernatant was transferred to separate Eppendorf tubes. Tubes were centrifuged at 15000 rpm for 20 minutes at 4° C. 30 μL of the supernatant was transferred to separate Eppendorf tubes with SDS buffer. Samples were loaded and soluble p110α was probed by Western blotting across the temperature gradient with anti-p110α antibody to determine the temperature at which p110α becomes insoluble. For quantification, densitometry was performed using ImageJ (Isakoff et al., Cancer Res 65, 10992-11000 (2005).) Western blot densitometry measurements were normalized to the densitometry of the lowest temperature point (46°), curves were fit to a Boltzmann sigmoid function, and melting temperatures (Tm (50%)) were determined.

Liposome Preparation and Liposome Binding Assays

PS, PE, and PI were purchased (Avanti) and cholesterol was purchased (Nu Chek Prep). Anionic lipid stocks were prepared at 10 mg/mL in HPLC-grade chloroform from using molar percentages of 35% PE, 25% PS, 5% PI, and 35% cholesterol. PIP2 lipid stocks were prepared at 35% PE, 25% PS, 4.9% PI, 0.1% PIP2, and 35% cholesterol. A gentle stream of argon gas was applied for 15 seconds and tubes were frozen and stored at −20° C. Prior to experiments, the lipid stocks were vortexed and 100 μL of chloroform (HPLC-grade) was transferred to a clean glass vial. Argon gas was immediately applied to the stock tube, capped, and stored at −20° C. Argon gas was applied the 100 μL aliquot leaving a translucent lipid film. 2 mL of 1× filter-sterilized TBSM buffer (50 mM Tris pH 8.0, 50 mM NaCl, 5 mM MgCl2) was added and lipids were hydrated at room temperature for 1 hour. Liposomes were extruded using a Mini-Extruder kit (Avanti) through an 0.8 μm membrane 15 times. Liposomes were transferred to a clean Eppendorf tube and centrifuged at 15000 rpm for 8 minutes. Supernatant was discarded, and the lipid pellet was resuspended in 100 μL TBSM buffer vigorously until resuspended. 900 μL of TBSM was added for a final volume of 1 mL. Differential light scattering was performed to assess size of the liposome population. 1 μg of PI3K complex in PBS was added to 70 μL liposomes (10 mg/mL) in a total volume of 100 μL. Binding reactions proceeded for 30 minutes at room temperature. Solutions were centrifuged at 15000 rpm for 15 minutes and supernatant was removed by aspiration. Lipid pellets were mixed with 50 μL SDS buffer, and the amount of bound p110α was probed by Western blotting. For quantification, densitometry was performed using ImageJ (Isakoff et al., Cancer Res 65, 10992-11000 (2005) and measurements were normalized to the densitometry of WT PI3K.

Lipid Kinase Assays

For triplicate kinase reactions, radioactive ATP buffer, protein, and PIP2 master mixes were assembled. The radioactive ATP buffer master mix contained 1100 μL 5× Assay Buffer I (SignalChem), 55 μL ATP (10 mM), 55 μL BSA (2 mg/mL), 55 μL 32P-labeled ATP (0.01 mCi/uL), and 2805 μL distilled water. The protein master mix contained 4 μg PI3K complex in 16 μL total volume. The PIP2 master mix contained 50 μL PIP2 (Avanti) and 450 μL distilled water. For each construct, 296 μL buffer master mix was combined with 14 μL protein master mix (buffer+ protein master mix) and was mixed well by pipetting. 90 μL of the buffer+ protein master mix was aliquoted in triplicate, corresponding to a total amount of 1.016 μg PI3K complex per reaction. To this was added 10 μL of PIP2 master mix (100 uL total volume per reaction) and the solution was mixed well by pipetting to start the reaction. Kinase reactions proceeded at 30° C. for 10 minutes. 50 μL of 4N HCL was added to quench the reaction followed by 100 μL of 1:1 methanol-chloroform. Tubes were vortexed for 30 seconds each and centrifuged at 15000 rpm for 10 minutes. Using gel loading pipet tips pipetted with chloroform in and out, 20 μL of the bottom hydrophobic phase was removed and spotted onto a TLC plate (EMD Millipore, M1164870001). Plates were placed in a sealed chamber with 65:35 1-propanol and 2M acetic acid and TLC was run overnight. Plates were exposed to a phosphor screen for 4 hours and imaged on a Typhoon FLA 7000.

IC50 Determination of Recombinant PI3K

The present disclosure used the Transcreener ADP2 fluorescence intensity assay (Bellbook Labs) to determine IC50 for recombinant PI3Kα. A standard curve was prepared with varied concentrations of ATP and ADP (100 μM total of nucleotide). Enzyme titrations were performed, and enzyme concentrations were chosen within the EC50-EC80 range for fluorescence. Kinase reactions were prepared in 384 well low volume black round bottom polystyrene NBS microplates (Corning #5414). 10 μL kinase reactions were prepared by combining PI3K with 1 uL alpelisib for 30 minutes at room temperature then adding ATP and diC8-PIP2 (Avanti) in kinase buffer at 30° C. for 1 hour. Final concentrations of reagents were 0-10 μM alpelisib, 100 μM ATP, 50 μM diC8-PIP2, and in the kinase buffer, 50 mM HEPES (pH 7.5), 4 mM MgCl2, 1% DMSO, and 0.01% Brij-35. Reactions were quenched by adding 10 μL of a mixture containing ADP2 antibody mixture and Alexa Fluor 594 Tracer. Detection of ADP fluorescence intensity was measured with a Phera Star plate reader (BMG Labtech) at excitation 584 nM, emission 620 nM, and gain adjustment of 2500. Data were analyzed by the GraphPad Prism software.

Michaelis Menten Kinetic Assays

The present disclosure adapted the Transcreener ADP2 fluorescence intensity assay (Bellbook Labs). 20 μL kinase reactions were prepared by adding ATP, diC8-PIP2, ADP2 antibody mixture, Alexa Fluor 594 Tracer, with and without PDGFR bis-phosphorylated peptide in kinase buffer in the absence of EDTA. PI3K was added to start the reaction. Final concentrations were 0-100 μM ATP, 0-50 μM diC8-PIP2, and 10 μM phosphopeptide. Serial fluorescence measurements were performed every 10 minutes for 2 hours with a Phera Star plate reader (BMG Labtech) at 30° C. at excitation 584 nM, emission 620 nM, and gain adjustment of 2500. Data were analyzed by the GraphPad Prism software.

Cell Viability Assays.

1000 MCF10A cells were seeded in 100 μL of MCF10A media (containing 2% horse serum) lacking EGF or insulin, per well, in a 96-well plate. 24 hours later, serial concentrations of alpelisib or GDC-0077 were added in 100 μL of MCF10A media (containing 2% horse serum) lacking EGF or insulin. Cells were incubated for 4 days and then developed with CellTiter-Glo (Promega). Fraction of cell viability was calculated relative to cell growth condition without drug.

Clinical Data Analysis from Phase 1 Clinical Trial

For analysis of natural history of double PIK3CA mutant breast cancer patients, clinical characteristics were analyzed from METABRIC and a prior cohort of curated metastatic patients (Razavi et al., Cancer Cell 34, 427-438 e426 (2018)).

Retrospective PFS analysis was performed on tumors from a large breast cancer dataset (n=1918) sequenced by MSK-IMPACT ((Razavi et al., Cancer Cell 34, 427-438 e426 (2018)). Tumors were included in analysis if both pre- and post-endocrine therapy (aromatase inhibitor or fulvestrant) biopsies confirmed WT, single PIK3CA mutation, or multiple PIK3CA mutations. Kaplan-Meier curves were generated for PFS after firstline aromatase inhibitor or firstline fulvestrant therapy. PFS analysis was performed on patients enrolled in NCT01870505, a phase 1 clinical trial of alpelisib plus letrozole or exemestane for patients with hormone-receptor positive locally-advanced unresectable or metastatic breast cancer. 46/51 patients had biopsy samples that confirmed PIK3CA mutant or WT alleles by tumor NGS, and these 46 patients were included in the final analysis.

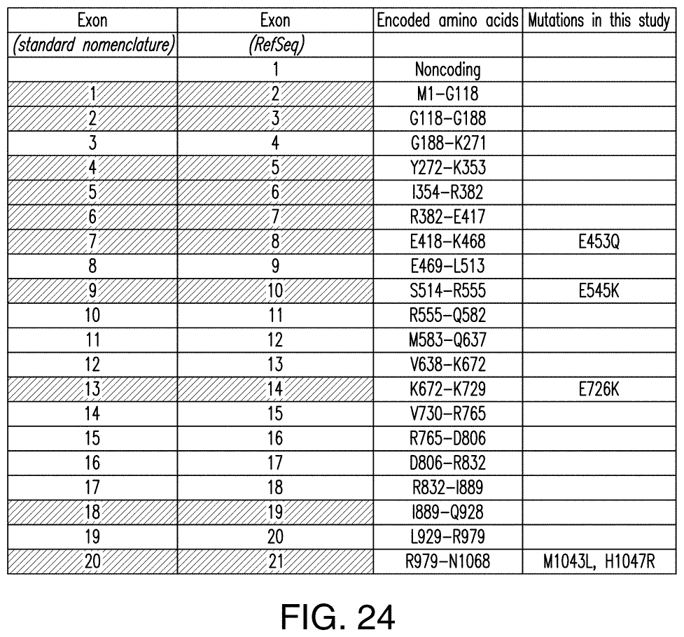

For analysis of the SANDPIPER clinical trial (Baselga, Journal of Clinical Oncology, 2018, 36, no. 18_suppl.) patient ctDNA samples (n=631), 508 patient samples met quality control parameters and were analyzed by Foundation Medicine One Liquid assay (Clark et al., J Mol Diagn 20, 686-702 (2018)) which sequences half the exons of PIK3CA and can detect mutations at amino acid positions 545, 1047, 453, 726, and 1043 (FIG. 24). 339 samples were identified with PIK3CA mutations, of which 66 contained two or more PIK3CA mutations. Patients with measurable disease from the ctDNA PIK3CA mutant cohort, on the taselisib arm, were analyzed based on the percentage change in the sum of longest diameter (SLD) of target lesion from baseline, and were tabulated by waterfall plot. Patients with both measurable and nonmeasurable disease from the ctDNA PIK3CA mutant cohort were assessed on the placebo and taselisib arms for overall response rate (defined as tumor shrinkage >30%). 95% CI for rates were constructed using the Blyth-Still-Casella method. The CI for the difference in ORRs between the two treatment arms were determined using the normal approximation to the binomial distribution. Response rates in the treatment arms were compared (p-value) using the stratified Cochran-Mantel-Haenszel test.

Statistical Analysis

All statistical analyses are shown in the appropriate method and figure legend. Investigators were unblinded when assessing the outcome of the in vivo experiments. All cellular and biochemical experiments were repeated at least three times unless otherwise indicated.

Although the presently disclosed subject matter and its advantages have been described in detail, it should be understood that various changes, substitutions and alterations can be made herein without departing from the spirit and scope of the invention. Moreover, the scope of the present application is not intended to be limited to the particular embodiments of the process, machine, manufacture, and composition of matter, means, methods and steps described in the specification. As one of ordinary skill in the art will readily appreciate from the invention of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Various patents, patent applications, publications, product descriptions, protocols, and sequence accession numbers are cited throughout this application, the inventions of which are incorporated herein by reference in their entireties for all purposes.

What is claimed is:

1. A method of treating a subject suffering from a cancer, the method comprising:
(a) identifying a subject as more likely to responsive to a PI3K inhibitor by a method comprising determining the presence of two or more PIK3CA mutations in a sample from the subject, wherein the presence of the two or more PIK3CA mutations indicates that the subject is more likely to be responsive to a PI3K inhibitor; and
(b) administering a PI3K inhibitor to the subject identified in (a) as more likely to responsive to a PI3K inhibitor.

2. The method of claim 1, wherein the cancer is selected from the group consisting of biliary tree cancer, hepatocellular carcinoma, cancers of the head and neck, gastric cancer, endometrial carcinoma, breast cancer, brain cancer, colorectal cancer, uterine cancer, bladder cancer, lung cancer, liver cancer, glioma, head and neck cancers, stomach cancer, cervical cancer, prostate cancer, prostate adenoma, melanoma, cutaneous melanoma, upper tract urothelial cancers, esophageal cancer, esophageal squamous cell carcinoma, esophageal adenocarcinoma, cutaneous squamous cell cancers, rectal cancer, rectal adenoma, ampullary cancer, cancer of unknown primary, oropharynx squamous cell cancer, intrahepatic cholangiocarcinoma, cholangiocarcinoma, esophagogastric adenocarcinoma, mucinous carcinoma, anaplastic astrocytoma, astrocytoma, kidney cancer, papillary renal cell carcinoma, ovarian cancer, high-grade serous ovarian cancer, poorly differentiated thyroid cancer, thyroid cancer, nasopharyngeal cancer, medulloblastoma, salivary duct cancer, non-seminomatous germ cell tumor, basaloid penile squamous cell cancer, and penile cancer.

3. The method of claim 1, wherein the cancer is a breast cancer.

4. The method of claim 1, wherein the cancer is an estrogen receptor-positive metastatic breast cancer.

5. The method of claim 1, wherein the two or more PIK3CA mutations are selected from Tables 4 and 5.

6. The method of claim 1, wherein the two or more PIK3CA mutations comprise a first PIK3CA mutation and a second PIK3CA mutation.

7. The method of claim 6, wherein the first PIK3CA mutation is selected from Tables 4 and 5; and/or the second PIK3CA mutation is selected from Tables 4 and 5.

8. The method of claim 6, wherein the first PIK3CA mutation is selected from the group consisting of E542, E545, and H1047; and/or the second PIK3CA mutation is selected from the group consisting of E453, E726, and M1043.

9. The method of claim 6, wherein the first PIK3CA mutation is selected from the group consisting of E542K, E545K, and H1047R; and/or the second PIK3CA mutation is selected from the group consisting of E453Q, E453K, E726K, M1043I, and M1043L.

10. The method of claim 6, wherein
a) the first PIK3CA mutation is H1047R and the second PIK3CA mutation is E453Q or E453K;
b) the first PIK3CA mutation is H1047R and the second PIK3CA mutation is E726K;
c) the first PIK3CA mutation is E545K and the second PIK3CA mutation is E726K;
d) the first PIK3CA mutation is E545K and the second PIK3CA mutation is M1043L or M1043I;
e) the first PIK3CA mutation is E545K and the second PIK3CA mutation is E453Q or E453K;
f) the first PIK3CA mutation is E542K and the second PIK3CA mutation is E726K;
g) the first PIK3CA mutation is E542K and the second PIK3CA mutation is M1043L or M1043I; or
h) the first PIK3CA mutation is E542K and the second PIK3CA mutation is E453Q or E453K.

11. The method of claim 1, wherein the presence of two or more PIK3CA mutations in the sample is determined by polymerase chain reaction.

12. The method of claim 1, wherein the sample is a plasma sample.

13. The method of claim 12, wherein the plasma sample comprises circulating tumor DNA.

14. The method of claim 1, wherein the sample is a sample of the cancer.

15. The method of claim 1, wherein the PI3K inhibitor is selected from the group consisting of BYL719, INK-1114, INK-1117, NVP-BYL719, SRX2523, LY294002, PIK-75, PKI-587, A66, CH5132799, GDC-0032 (taselisib), GDC-0077, and combinations thereof.

16. The method of claim 1, wherein the PI3K inhibitor is BYL719 or GDC-0032.

17. A kit for determining the responsiveness of a cancer cell or a subject suffering from a cancer to a PI3K inhibitor, wherein the kit comprises a means for detecting two or more PIK3CA mutations, wherein the means comprises determining the presence of two or more PIK3CA mutations in a sample from the subject, wherein the presence of the two or more PIK3CA mutations indicates that the subject is more likely to be responsive to a PI3K inhibitor.

18. A kit for identifying a subject suffering from a cancer as more likely to respond to a PI3K inhibitor, wherein the kit comprises a means for detecting two or more PIK3CA mutations, wherein the means comprises determining the presence of two or more PIK3CA mutations in a sample from the subject, wherein the presence of the two or more PIK3CA mutations indicates that the subject is more likely to be responsive to a PI3K inhibitor.
